(19) United States
(12) Patent Application Publication
(10) Pub. No.: US 2024/0218330 A1
(43) Pub. Date: Jul. 4, 2024

(54) COMPOSITIONS FOR IMPROVING THE TRANSDUCTION OF CELLS BY VIRAL VECTORS

(71) Applicants: Ospedale San Raffaele S.r.l., Milan (IT); Fondazione Telethon, Rome (IT)

(72) Inventors: Anna Christina Kajaste-Rudnitski, Milan (IT); Erika Valeri, Milan (IT); Giulia Unali, Milan (IT); Francesco Piras, Milan (IT)

(21) Appl. No.: 18/288,768

(22) PCT Filed: Apr. 26, 2022
(86) PCT No.: PCT/EP2022/061114

§ 371 Date: Oct. 27, 2023

(30) Foreign Application Priority Data
Apr. 28, 2021 (AU) 2021202658

(51) Int. Cl. C12N 5/0789; A61K 31/513; A61K 31/7072; A61K 35/28; A61K 38/13; C12N 15/86

(52) U.S. Cl. CPC C12N 5/0647; A61K 31/513; A61K 31/7072; A61K 35/28; A61K 38/13; C12N 15/86; C12N 2500/40; C12N 2501/04; C12N 2510/00; C12N 2740/15043

(57) ABSTRACT

A combination of: (a) at least one deoxyribonucleoside (dN) or a derivative thereof and cyclosporin H (CsH) or a derivative thereof: or (b) at least one pyrimidine precursor and cyclosporin H (CsH) or a derivative thereof.

### FIELD OF THE INVENTION

The present invention relates to compounds for improving the transduction of cells by viral vectors and/or improving gene editing of cells.

### BACKGROUND TO THE INVENTION

Ex-vivo gene therapies using hematopoietic stem and/or progenitor cells (HSPCs) have become effective therapeutic options for the treatment of several monogenic diseases including blood and metabolic disorders. Autologous HSPCs corrected using lentiviral and gamma-retroviral vectors have reached the clinic, paving the way for HSPC gene therapy as the preferred treatment option over HSPC allogeneic transplantations in certain contexts.

However, current HSPC gene therapy protocols require the use of multiple hits at high vector doses and prolonged ex vivo culture to reach clinically relevant transduction levels, imposing large-scale vector production and potentially compromising HSPC preservation in culture. For example, extensively cultured HSPCs may fail to maintain multipotency, showing rapid and progressive loss of stem cell activity required for good engraftment and homing capacity (Barquinero J, et al. (2000) Blood 95: 3085-3093; Glimm H, et al. (2000) Blood 96: 4185-4193; Millington M, et al. (2009) PLOS One 4: e6461). This underscores the need for protocols which combine high levels of gene transfer with minimal HSPC manipulation.

Unstimulated HSPC may represent the ideal gene therapy target, but their quiescence and the presence of enhanced expression of innate restriction factors acting at different steps of the transduction pathway render them particularly resistant to lentiviral gene transfer (Santoni de Sio F R, et al. (2008) Stem Cells 26: 2142-2152). Other quiescent blood cells such as macrophages and resting T cells are also highly refractory to lentiviral gene transfer. In particular, in the context of CAR T cell-based cancer immunotherapies, the resting T stem memory compartment remains difficult to target during CAR T cell generation.

### SUMMARY OF THE INVENTION

The inventors have surprisingly found that exogenous delivery of deoxyribonucleosides (dNs) boosts lentiviral transduction of unstimulated HSPCs. The inventors have surprisingly found a pivotal role of pyrimidines, in particular of deoxycytidines, in restricting lentiviral gene transfer and editing into quiescent HSPCs that does not seem to depend on intracellular levels of dCTP. Moreover, the inventors have surprisingly found a significant synergistic effect of dNs with CsH. A combination of dNs and CsH also significantly improves lentiviral transduction efficiencies in HSPCs and primary human resting T cells.

In one aspect, the invention provides a combination of at least one deoxyribonucleoside (dN) or a derivative thereof and at least one additional transduction enhancer (e.g. cyclosporin H (CsH) or a derivative thereof). In one aspect, the invention provides a combination of at least one pyrimidine precursor and at least one additional transduction enhancer (e.g. cyclosporin H (CsH) or a derivative thereof). In preferred embodiments, the at least one additional transduction enhancer comprises or consists of CsH or a derivative thereof.

In one aspect, the invention provides a combination of at least one deoxyribonucleoside (dN) or a derivative thereof and cyclosporin H (CsH) or a derivative thereof. In one aspect, the invention provides a combination of at least one pyrimidine precursor and cyclosporin H (CsH) or a derivative thereof.

In one embodiment, the combination is for cell transduction. In one embodiment, the combination is for improving the transduction of cells by viral vectors and/or improving gene editing of cells.

In one embodiment, the at least one deoxyribonucleoside (dN) or a derivative thereof comprises or consists of at least one pyrimidine dN or a derivative thereof. In one embodiment, the at least one dN or a derivative thereof comprises or consists of deoxycytidine (dC) or a derivative thereof and/or thymidine (dT) or a derivative thereof. In one embodiment, the at least one dN or a derivative thereof comprises or consists of dC or a derivative thereof.

In one embodiment, the at least one dN or a derivative thereof further comprises at least one purine dN or a derivative thereof. In one embodiment, the at least one dN or a derivative thereof further comprises deoxyadenosine (dA) or a derivative thereof and/or deoxyguanosine (dG) or a derivative thereof.

In one embodiment, the at least one dN or a derivative thereof comprises or consists of at least one purine dN or a derivative thereof. In one embodiment, the at least one dN or a derivative thereof comprises or consists of deoxyadenosine (dA) or a derivative thereof and/or deoxyguanosine (dG) or a derivative thereof.

In one embodiment, the at least one dN or a derivative thereof comprises or consists of dC or a derivative thereof, dT or a derivative thereof, dA or a derivative thereof, and dG or a derivative thereof.

The pyrimidine precursor may be, for example, orotic acid (OA), orotidine 5′-monophosphate (OMP), uridine 5′-monophosphate (UMP), uridine 5′-diphosphate (UDP) or uridine 5′-triphosphate (UTP).

In one embodiment, the at least one pyrimidine precursor comprises or consists of orotic acid (OA). In one embodiment, the at least one pyrimidine precursor comprises or consists of uridine 5′-monophosphate (UMP).

In one embodiment, at least one dN or a derivative thereof and CsH or a derivative thereof are in a dN:CsH molar ratio of from about 1:2 to about 1000:1. In one embodiment, at least one dN or a derivative thereof and CsH or a derivative thereof are in a dN:CsH molar ratio of from about 2:1 to about 200:1, preferably wherein at least one dN or a derivative thereof and CsH or a derivative thereof are in a dN:CsH molar ratio of from about 10:1 to about 100:1.

In one embodiment, at least one pyrimidine dN or a derivative thereof and CsH or a derivative thereof are in a dN:CsH molar ratio of from about 1:2 to about 1000:1. In one embodiment, at least one pyrimidine dN or a derivative thereof and CsH or a derivative thereof are in a dN:CsH molar ratio of from about 2:1 to about 200:1, preferably wherein at least one pyrimidine dN or a derivative thereof and CsH or a derivative thereof are in a dN:CsH molar ratio of from about 10:1 to about 100:1.

In one embodiment, dC or a derivative thereof and CsH or a derivative thereof are in a dN:CsH molar ratio of from about 1:2 to about 1000:1 and/or dT or a derivative thereof and CsH or a derivative thereof are in a molar dN:CsH ratio of from about 1:2 to about 1000:1. In one embodiment, dC or a derivative thereof and CsH or a derivative thereof are in a dN:CsH molar ratio of from about 1:2 to about 1000:1. In one embodiment, dT or a derivative thereof and CsH or a derivative thereof are in a molar dN:CsH ratio of from about 1:2 to about 1000:1. In one embodiment, dC or a derivative thereof and CsH or a derivative thereof are in a dN:CsH molar ratio of from about 2:1 to about 200:1 or from about 10:1 to about 100:1 and/or dT or a derivative thereof and CsH or a derivative thereof are in a molar dN:CsH ratio of from about 2:1 to about 200:1 or from about 10:1 to about 100:1. In one embodiment, dC or a derivative thereof and CsH or a derivative thereof are in a dN:CsH molar ratio of from about 2:1 to about 200:1 and dT or a derivative thereof and CsH or a derivative thereof are in a molar dN:CsH ratio of from about 2:1 to about 200:1. In one embodiment, dC or a derivative thereof and CsH or a derivative thereof are in a dN:CsH molar ratio of from about 10:1 to about 100:1 and dT or a derivative thereof and CsH or a derivative thereof are in a molar dN:CsH ratio of from about 10:1 to about 100:1. In one embodiment, dC or a derivative thereof and CsH or a derivative thereof are in a dN:CsH molar ratio of from about 2:1 to about 200:1, preferably from about 10:1 to about 100:1. In one embodiment, dT or a derivative thereof and CsH or a derivative thereof are in a dN:CsH molar ratio of from about 2:1 to about 200:1, preferably from about 10:1 to about 100:1.

In one embodiment, dA or a derivative thereof and/or dG or a derivative thereof are in a dN:CsH molar ratio of from about 1:2 to about 1000:1. In one embodiment, dA or a derivative thereof and CsH or a derivative thereof are in a dN:CsH molar ratio of from about 1:2 to about 1000:1. In one embodiment, dG or a derivative thereof and CsH or a derivative thereof are in a molar dN:CsH ratio of from about 1:2 to about 1000:1. In one embodiment, dA or a derivative thereof and/or dG or a derivative thereof are in a dN:CsH molar ratio of from about 2:1 to about 200:1, preferably wherein the dA or a derivative thereof and/or dG or a derivative thereof are in a dN:CsH molar ratio of from about 10:1 to about 100:1. In one embodiment, dA or a derivative thereof and CsH or a derivative thereof are in a dN:CsH molar ratio of from about 2:1 to about 200:1, preferably from about 10:1 to about 100:1. In one embodiment, dG or a derivative thereof and CsH or a derivative thereof are in a dN:CsH molar ratio of from about 2:1 to about 200:1, preferably from about 10:1 to about 100:1.

In one embodiment, at least one dN or a derivative thereof is at a concentration of from about 25 μM to about 1000 μM, preferably from about 100 μM to about 500 μM. In one embodiment, at least one pyrimidine dN or a derivative thereof is at a concentration of from about 25 μM to about 1000 μM, preferably from about 100 μM to about 500 μM. In one embodiment, dC or a derivative thereof is at a concentration of from about 25 μM to about 1000 μM, preferably from about 100 μM to about 500 μM, and/or dT or a derivative thereof is at a concentration of from about 25 μM to about 1000 μM, preferably from about 100 μM to about 500 μM. In one embodiment, dA or a derivative thereof is at a concentration of from about 25 μM to about 1000 μM, preferably from about 100 μM to about 500 μM, and/or dG or a derivative thereof is at a concentration of from about 25 μM to about 1000 μM, preferably from about 100 μM to about 500 μM. In one embodiment, dC or a derivative thereof is at a concentration of from about 25 μM to about 1000 μM, dT or a derivative thereof is at a concentration of from about 25 μM to about 1000 μM, dA or a derivative thereof is at a concentration of from about 25 μM to about 1000 μM, and dG or a derivative thereof is at a concentration of from about 25 μM to about 1000 μM. In one embodiment, dC or a derivative thereof is at a concentration of from about 100 μM to about 500 μM, dT or a derivative thereof is at a concentration of from about 100 μM to about 500 μM, dA or a derivative thereof is at a concentration of from about 100 μM to about 500 μM, and dG or a derivative thereof is at a concentration of from about 100 μM to about 500 μM.

In one embodiment, the CsH or derivative thereof is at a concentration of from about 1 to about 50 μM. In one embodiment, the CsH or derivative thereof is at a concentration of from about 5 μM to about 50 μM. In one embodiment, the CsH or derivative thereof is at a concentration of from about 10 μM to about 50 μM. In one embodiment, the CsH or derivative thereof is at a concentration of from about 1 to about 25 μM. In one embodiment, the CsH or derivative thereof is at a concentration of from about 1 to about 10 μM. In one embodiment, the CsH or derivative thereof is at a concentration of from about 5 to about 10 μM.

The combination may be, for example, in the form of a product, composition or kit. one aspect, the present invention provides a composition comprising a combination 10 according to the present invention. In one embodiment, the composition is a cell medium or a media supplement, preferably wherein the composition is a cell medium.

In one aspect, the present invention provides a kit comprising a combination according to the present invention or a composition according to the present invention and, optionally, one or more further compositions for cell transduction and/or optionally one or more further agents or cell transduction.

In one aspect, the present invention provides use of a combination according to the present invention, a composition according to the present invention, or a kit according to the present invention, for increasing the efficiency of transduction of an isolated population of cells by a viral vector and/or increasing the efficiency of gene editing of an isolated population of cells when transduced by a viral vector.

In one aspect, the present invention provides a method of transducing a population of cells comprising the steps of:
(a) contacting the population of cells with a combination according to the present invention or a composition according to the present invention; and
(b) transducing the population of cells with a viral vector.

In one embodiment, the method is an in vitro method or an ex vivo method.

In one aspect, the present invention provides use of a deoxyribonucleoside (dN) or a derivative thereof for increasing the efficiency of transduction of an isolated population of cells by a viral vector and/or increasing the efficiency of gene editing of an isolated population of cells when transduced by a viral vector.

In one aspect, the present invention provides use of a pyrimidine precursor for increasing the efficiency of transduction of an isolated population of cells by a viral vector and/or increasing the efficiency of gene editing of an isolated population of cells when transduced by a viral vector.

In one aspect, the present invention provides a method of transducing a population of cells comprising the steps of:
(a) contacting the population of cells with at least one deoxyribonucleoside (dN) or a derivative thereof; and
(b) transducing the population of cells with a viral vector.

In one aspect, the present invention provides a method of transducing a population of cells comprising the steps of:
(a) contacting the population of cells with at least one pyrimidine precursor; and
(b) transducing the population of cells with a viral vector.

In a preferred embodiment, the population of cells comprises or consists substantially of: (i) unstimulated haematopoietic stem and/or progenitor cells (HSPCs); and/or (ii) CD14⁻ peripheral blood mononuclear cells (PBMCs)

In one embodiment, the method is an in vitro method or an ex vivo method. In one embodiment the method increases the efficiency of transduction and/or increases the efficiency of gene editing.

In one embodiment, the dN or a derivative thereof is a pyrimidine dN or a derivative thereof. In one embodiment, the dN or a derivative thereof is deoxycytidine (dC) or a derivative thereof, and/or thymidine (dT) or a derivative thereof, preferably wherein the dN or a derivative thereof is dC or a derivative thereof. In one embodiment, the dN or a derivative thereof is at a concentration of from about 25 μM to about 1000 μM, preferably wherein the dN or a derivative thereof is at a concentration of from about 100 μM to about 500 μM.

In one embodiment, the population of cells is contacted with the dN or a derivative thereof in combination with CsH or a derivative thereof. In one embodiment, the CsH or derivative thereof is at a concentration of from about 1 μM to about 50 μM. In one embodiment, the CsH or derivative thereof is at a concentration of from about 5 μM to about 50 μM. In one embodiment, the CsH or derivative thereof is at a concentration of from about 10 μM to about 50 μM.

In one embodiment, the population of cells is contacted with the pyrimidine precursor in combination with CsH or a derivative thereof. In one embodiment, the population of cells is contacted with orotic acid (OA) in combination with CsH or a derivative thereof. In one embodiment, the population of cells is contacted with uridine 5′-monophosphate (UMP) in combination with CsH or a derivative thereof.

In one embodiment, the dN or a derivative thereof is a purine dN or a derivative thereof.

In one embodiment, the population of cells is further contacted with a purine dN or a derivative thereof. In one embodiment, the purine dN or a derivative thereof is at a concentration of from about 25 μM to about 1000 μM, preferably wherein the purine dN or a derivative thereof is at a concentration of from about 100 μM to about 500 μM.

In preferred embodiments, the cells are human cells or mouse cells, preferably human cells.

In some embodiments, the method comprises two steps of transducing the population of cells with a viral vector. In some embodiments, the method comprises only one step of transducing the population of cells with a viral vector. The population of cells may, for example, not be subjected to any further transduction with a viral vector, for example before administering the transduced cells to a subject.

In some embodiments, the total culture time of the cells is about 36 to 72 hours. In some embodiments, the total culture time of the cells is about 48 to 72 hours. In some embodiments, the total culture time of the cells is about 36 to 60 hours. In some embodiments, the total culture time of the cells is about 42 to 54 hours. In some embodiments, the total culture time of the cells is about 48 hours.

In some embodiments, the total culture time of the cells is about 42 to 78 hours. In some embodiments, the total culture time of the cells is about 54 to 78 hours. In some embodiments, the total culture time of the cells is about 42 to 66 hours. In some embodiments, the total culture time of the cells is about 48 to 60 hours. In some embodiments, the total culture time of the cells is about 54 hours.

In preferred embodiments, the cells are unstimulated.

In preferred embodiments, the cells are quiescent.

In one embodiment, the population of cells comprises or consists substantially of:
(i) haematopoietic stem and/or progenitor cells (HSPCs); and/or
(ii) CD14⁻ peripheral blood mononuclear cells (PBMCs).

In one embodiment, the method further comprises a step of enriching the population of cells for the HSPCs or CD14⁻ PBMCs. The population of cells may be an isolated population of cells.

In one embodiment, the HSPCs are unstimulated HSPCs. In one embodiment, the HSPCs are CD34⁺ or CD34⁻ cells. In one embodiment, the HSPCs are CD34⁺ cells, preferably wherein the HSPCs are CD34⁺CD133⁻CD90⁻, CD34⁺CD133⁺CD90⁻, or CD³⁴⁺CD133⁺CD90⁺ cells, more preferably wherein the HSPCs are CD34⁺CD133⁺CD90⁺ cells. In one embodiment, the PBMCs are CD3⁺, CD4⁺, and/or CD8⁺ T cells.

In one embodiment, the T cells are CD45RA+CD62L+CD95− T cells (e.g. T naïve, TN) or CD45RA+CD62L+CD95+ T cells (T stem cell memory, TSCM), or a mixed population thereof.

In one embodiment, the T cells are further contacted with IL7 and/or IL15.

In one embodiment, the viral vector is a retroviral vector, preferably a lentiviral vector. In one embodiment, the lentiviral vector is an integration-defective lentiviral vector (e.g. an integrase-defective lentiviral vector). In one embodiment, the viral vector is pseudotyped to enter cells via an endocytosis-dependent mechanism and/or the viral vector is a VSV-g pseudotyped vector. In one embodiment, the viral vector is a measles virus glycoprotein pseudotyped viral vector. In one embodiment, the viral vector is pseudotyped with measles virus glycoproteins hemagglutinin (H) and fusion protein (F).

In one embodiment, the percentage of cells transduced by the vector is increased and/or the vector copy number per cell is increased.

In one aspect, the present invention provides a method of gene therapy comprising the steps of:
(a) transducing a population of cells according to the method of the present invention; and
(b) administering the transduced cells to a subject.

In one embodiment, the transduced cells are administered to a subject as part of an autologous stem cell transplant procedure and/or an allogeneic stem cell transplant procedure.

In one aspect, the present invention provides a population of cells prepared according to the method of the present invention. The population of cells may be an isolated population of cells.

In one aspect, the present invention provides a pharmaceutical composition comprising the population of cells of the present invention.

In one aspect, the present invention provides a population of cells of the present invention for use in therapy. In one embodiment, the population is administered as part of an autologous stem cell transplant procedure or an allogeneic stem cell transplant procedure.

In one aspect, the present invention provides a combination according to the present invention, a composition according to the present invention, or a kit according to the present invention, for use in gene or cell therapy.

In one aspect, the present invention provides a deoxyribonucleoside (dN) or a derivative thereof for use in gene or cell therapy.

In one aspect, the present invention provides a pyrimidine precursor for use in gene or cell therapy.

### DESCRIPTION OF DRAWINGS

FIG. 1—Exogenous deoxynucleosides (dNs) synergize with cyclosporin H (CsH) to significantly improve lentiviral transduction and gene editing in quiescent HSPC

(A) Unstimulated hHSPC were pre-treated with deoxyribonucleotide triphosphates (dNTPs) before transduction with a lentiviral vector (LV) in the presence of CsH. Percentages of transduced cells were assessed at 5 days post-transduction (mean±SEM, n=2). (B) Unstimulated hHSPC were pre-treated with a mixture of the 4 deoxynucleosides (dNs) at a final concentration of 500 μM each before transduction with a LV in presence of CsH. Percentages of transduced cells were assessed at 5 days post-transduction and expressed as fold increase of CsH+dNs/CsH control (mean±SEM, n=8, Wilcoxon signed rank test versus CsH=1, **p=0.0078). (C) Transduction efficiencies in the different subpopulations of unstimulated hHSPC expressed as fold increase of CsH+dNs/CsH control (mean±SEM, n=8, Wilcoxon signed rank test versus each CsH=1 **p=0.0078). (D) The composition of unstimulated hHSPC was evaluated 5 days post transduction. (E) Transduction efficiencies in unstimulated hHSPC±dNs±CsH expressed as fold increase versus DMSO control (mean±SEM, n=3). (F) Transduction efficiencies in the different subpopulations of unstimulated hHSPC±dNs±CsH. (G) The composition of unstimulated hHSPC was evaluated 5 days post transduction. (H) Transduction efficiency was evaluated in unstimulated hHSPC in presence of different concentrations of dNs and expressed as fold increase versus CsH only control (mean±SEM, n≥2). (I) Unstimulated hHSPC were transduced with an integrase defective LV (IDLV) at MOI=100±CsH in presence or not of dNs. Transduction efficiency was evaluated at 3 days post transduction (n=1). (J) Scheme of the gene editing protocol for unstimulated hHSPC. (K-L) Percentage of edited cells at AAVS1 locus measured within the bulk (K) or within the indicated HSPC subpopulations (L) 3 days post editing (n=1). (M) The composition of unstimulated hHSPC was evaluated 3 days post editing. (N) Transduction efficiencies in stimulated hHSPC±dNs±CsH (n=1).

FIG. 2—Combination of cyclosporin H (CsH) and exogenous deoxynucleosides (dNs) improves transduction across species and in multiple quiescent hematopoietic cell types

(A) Unstimulated murine HSPC from WT or SAMHD1 KO mice were transduced with a LV (MOI=10)±8 μM CsH after exposure or not to dNs (mean±SEM, n=3). (B) Activated primary CD3⁺ T cells were transduced (MOI=1)±CsH after exposure or not to dNs (mean±SEM, n=4, Dunn's adjusted Kruskal-Wallis test; ns, not significant). (C) Peripheral blood mononuclear cells (PBMC), depleted of the CD14⁺ subset, were transduced with a LV (MOI=25-50)±CsH after exposure or not to dNs (mean±SEM, n=4, Dunn's adjusted Kruskal-Wallis test, **for p<0.01). (D) Transduction efficiency within the CD3⁺ T cells subset (mean±SEM, n=4, Dunn's adjusted Kruskal-Wallis test, **for p<0.01). (E) Transduction efficiency within the CD4⁺ and CD8⁺ T cells subsets (mean±SEM, n=4, Dunn's adjusted Kruskal-Wallis test, *for p<0.05, ** for p<0.01). (F-G) The composition of resting CD3⁺ T cells was evaluated 3 days post transduction. TSCM, Stem memory T cells; CM, Central Memory; EM, Effector Memory; TEMRA, Terminally differentiated effector memory. (H) Transduction efficiency within the CD3⁺ Stem memory T cells (TSCM) subset (mean±SEM, n=2, Tukey's adjusted Kruskal-Wallis test, *for p<0.05).

FIG. 3—Pyrimidine pools are limiting for lentiviral transduction in quiescent HSPC

(A-B) Unstimulated hHSPC were transduced in presence of CsH upon 4h pre-exposure to dA, dG or dA+dG mix. Percentages of transduced cells within the bulk (A) and in the different subpopulations of unstimulated hHSPC (B) expressed as fold increase versus CsH only control (mean±SEM, n≥1). (C-D) Unstimulated hHSPC were transduced in presence of CsH upon 4 h pre-exposure to dC, dT or dC+dT mix. Percentages of transduced cells within the bulk (C) and in the different subpopulations of unstimulated hHSPC (D) expressed as fold increase versus CsH only control (mean±SEM, n≥2). (E) Intracellular dNTP levels were measured in unstimulated hHSPC 24 h post exposure to dNs in presence or absence of CsH.

FIG. 4—Unstimulated HSPC engraft to higher extend despite lower cell input as compared to pre-stimulated counterparts

(A) Engraftment levels of mice, from groups of unstimulated versus stimulated human HSPC, in the peripheral blood (PB) at indicated weeks post-transplant and in the bone marrow (BM) at 20 weeks post-transplant (mean±SEM, n=10, 12 mice per group, Mann-Whitney test, **p=0.0035). (B) Experimental scheme of the transplantation experiment. HSPC were pre-stimulated 24 h with a cocktail of early active cytokines before transduction or kept unstimulated and transduced immediately with a LV (MOI=25)±CsH±dNs. Cells were then injected 20 h post transduction by T₀ equivalent into NSG mice. (C-D) In vitro transduction efficiency of the in vivo experiment was assessed 5 days post transduction in the bulk population of HSPC (C) and in the indicated subpopulations (D). (E) The composition of hHSPC was evaluated 5 days post transduction.

FIG. 5. Measles pseudotyped LV outperform the gold standard VSV-G LV significantly increasing transduction yields in resting CD14⁻ PBMC pretreated with dNs and CsH.

(A) Peripheral blood mononuclear cells (PBMC), depleted of the CD14⁺ subset, were transduced with a VSV-G LV (MOI=25) or Measles LV±CsH (mean±SEM, n=1). (B) Activated primary CD3⁺ T cells were transduced with either VSV-G LV (MOI=25) or Measles LV (MOI=10) in presence or absence of CsH (mean±SEM, n=1). (C)). (C) Peripheral blood mononuclear cells (PBMC), depleted of the CD14⁺ subset, with a VSV-G LV (MOI=25) or Measles LV±CsH after exposure or not to dNs (mean±SEM, n=1). (D-E) Subsets composition of resting CD3⁺ T cells was evaluated 3 days post transduction. TSCM, Stem memory T cells; CM, Central Memory; EM, Effector Memory; TEMRA, Terminally differentiated effector memory. (F-G) Transduction efficiency within the Naive CD3⁺ T cells subset (mean±SEM, n=1). (H-I) Transduction efficiency within the CD4⁺ and CD8⁺ T cells subsets (mean±SEM, n=1). (J-K) Resting CD3⁺ T cells cell composition (mean±SEM, n=1).

FIG. 6. Pyrimidine pools are limiting for lentiviral transduction in quiescent HSPC.

(A) Heatmaps showing the expression levels of Carbamoyl-P synthetase (CPS) and Dihydroorotate dehydrogenase (DHODH) analysed from publicly available dataset where the gene expression profile of quiescent long-term (qLT)-HSC is compared to the one of activated long term (aLT)-HSC. (B-C) Relative gene expression levels of CPS and DHODH in unstimulated and stimulated hHSPC from the same donors (mean±SEM, n=4, Mann Whitney test, *for p<0.05. (D) Unstimulated hHSPC were transduced with a LV±8 μM CsH after exposure or not to dNs or 7.5 μM Orotic Acid. Percentages of transduced cells expressed as fold increase versus DMSO control (mean±SEM, n=4, Dunn's adjusted Kruskal-Wallis test, *for p<0.05, **for p<0.01. (E) Unstimulated hHSPC were transduced with a LV±8 μM CsH after exposure or not to dNs or 1 mM UMP. Percentages of transduced cells expressed as fold increase versus DMSO control (mean±SEM, n=3, Dunn's adjusted Kruskal-Wallis test, *for p<0.05). (F-G) Unstimulated hHSPC were transduced with γRV or SIV (MOI=10)±CsH after exposure or not to dNs (mean±SEM, n=2 per γRV, n=3 per SIV, Dunn's adjusted Kruskal-Wallis test, *for p<0.05. (H) Unstimulated hHSPC were transduced with AAV6 (MOI=10000) after exposure or not to dNs (mean±SEM, n=3).

FIG. 7. dNs delivery does not affect the proliferation, apoptosis and cell cycle status of unstimulated hHSPC.

(A) Impact of dNs on cell proliferation was assessed in unstimulated hHSPC 24 h, 48 h and 5 days after dNs delivery during or not LV transduction (mean±SEM; n=4). (B) Impact of dNs on apoptosis was assessed in unstimulated hHSPC 48 hr after dNs delivery during or not LV transduction (mean±SEM; n=2-3). (C-D) The impact of dNs on cell-cycle status of unstimulated hHSPC was evaluated 24 h (C) and 5 days (D) after dNs delivery during or not of LV transduction (mean±SEM; n=4). (E) Colony-forming unit (CFU) output after 2 weeks of differentiation in unstimulated hHSPC exposed or not to dNs compared to stimulated hHSPC (mean±SEM; n=4).

FIG. 8. Unstimulated HSPC engraft similarly to their pre-stimulated counterpart, despite lower cellular input.

(A) Experimental scheme of the transplantation experiment. Human CD34⁺ cells from cord blood were pre-stimulated 24 h with a cocktail of early active cytokines before transduction with a LV (MOI=25) in presence of 8 μM CsH or kept unstimulated and transduced immediately with a LV (MOI=25) in presence of CsH and 500 μM mix of all dNs. Cells were then injected 20 h post transduction by T₀ equivalent into NSG mice. (B-C) In vitro transduction efficiency of the in vivo experiment was assessed 5 days post transduction in the bulk population of HSPC (B) and in the indicated subpopulations (C). (D) The composition of hHSPC was evaluated 5 days post transduction. (E) In vitro VCN/genome were measured 5 days post transduction. (F) N° of cells injected into mice for each experimental group was counted immediately before transplantation. (G) Engraftment levels in the peripheral blood of mice from the two experimental groups (mean±SD; n=8 mice per group, t-test, *for p<0.05, ns=non significant). (H) Engraftment levels in the bone marrow at 20 weeks post-transplant (mean±SD; n=8). (I) Percentages of hCD34⁺ cells within hCD45⁺ in the bone marrow compartment at 20 weeks (mean±SD; n=8). (J) Cell composition of the hCD34⁺ fraction in the bone marrow (mean±SD; n=8). (K) Engraftment levels in the spleen at 20 weeks post-transplant (mean±SD; n=8). (L) Percentages of different subpopulation from the spleen of recipient mice at 20 weeks (mean±SD; n=8). (M) Engraftment levels in the thymus at 20 weeks post-transplant (mean±SD; n=8). (N) Percentages of hCD3⁺ cells within hCD45⁺ in thymus at 20 weeks (mean±SD; n=8). (O) Transduction efficiencies in the peripheral blood of mice from the two experimental groups (mean±SD; n=8 mice per group). (P-R) Transduction efficiencies, measured as % of GFP⁺ cells, in bone marrow (P), spleen (Q) and thymus (R) at 20 weeks post-transplant (mean±SEM; n=8). (S-U) VCN/genome were measured in the bone marrow (S), spleen (T) and thymus (U) at 20 weeks (mean±SEM; n=8).

FIG. 9. dNs and CsH in unstimulated mPB HSPC allow to reach good level of transduction while preserving their repopulation capacity.

(A) Experimental scheme of the transplantation experiment. Human CD34⁺ cells from mobilised peripheral blood were pre-stimulated 24 h with a cocktail of early active cytokines before transduction with a LV (MOI=50) in presence of 8 μM CsH or kept unstimulated and transduced immediately with a LV (MOI=50) in presence of CsH and 500 uM mix of all dNs. Cells were then injected 20 h post transduction by T₀ equivalent into NSG mice. (B-C) In vitro transduction efficiency of the in vivo experiment was assessed 5 days post transduction in the bulk population of HSPC (B) and in the indicated subpopulations (C). (D) The composition of hHSPC was evaluated 5 days post transduction. (E) In vitro VCN/genome were measured 5 days post transduction. (F) N° of cells injected into mice for each experimental group was counted immediately before transplantation. (G) Engraftment levels in the peripheral blood of mice from the two experimental groups (mean±SD; n=3 mice per group, t-test, ns=non significant). (H) Engraftment levels in the bone marrow at 20 weeks post-transplant (mean±SD; n=3). (I) Transduction efficiencies in the peripheral blood of mice from the two experimental groups (mean±SD; n=3 mice per group). (J-L) Transduction efficiencies, measured as % of GFP⁺ cells, in bone marrow (J), spleen (K) and thymus (L) at 20 weeks post-transplant (mean±SEM; n=3). (M-O) VCN/genome were measured in the bone marrow (M), spleen (N) and thymus (O) at 20 weeks (mean±SEM; n=3).

### DETAILED DESCRIPTION

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Numeric ranges are inclusive of the numbers defining the range.

### Combination

In one aspect, the present invention provides a combination of at least one deoxyribonucleoside (dN) or a derivative thereof and at least one additional transduction enhancer (e.g. Cyclosporin H (CsH) or a derivative thereof). In one aspect, the invention provides a combination of at least one pyrimidine precursor and at least one additional transduction enhancer (e.g. cyclosporin H (CsH) or a derivative thereof).

The combination may be provided in any form, for example the at least one dN or a derivative thereof and the at least one additional transduction enhancer (e.g. CsH or a derivative thereof) may be combined in a composition, in a kit-of-parts, and or applied in combination.

The combination may be any combination suitable for cell culture, e.g. the combination may be applied to a population of cells before, during, before and during, and/or after cell culture or contact with a viral vector, or any combination thereof. In one embodiment, the combination may be applied before and/or during cell culture. In one embodiment, the combination is suitable for cell transduction e.g. the combination may be applied to a population of cells before, during, before and during, or after contact with a viral vector, or any combination thereof. In one embodiment, the combination may be applied before and/or during contact with a viral vector. The agents may be, for example, applied to the population of cells simultaneously, sequentially or separately. In one embodiment, the combination is for improving the transduction of cells by viral vectors and/or improving gene editing of cells.

### Deoxyribonucleosides

Deoxyribonucleosides (dNs, also known as 2′-deoxyribonucleosides) are composed of a nucleobase and 2′-deoxyribose. Exemplary dNs include deoxycytidine, thymidine, deoxyadenosine, deoxyguanosine, and deoxyuridine.

The nucleobase may be either a pyrimidine or a purine. Pyrimidine (also known as 1,3-Diazabenzene) is an aromatic heterocyclic organic compound similar to pyridine. Purine is a heterocyclic aromatic organic compound that consists of pyrimidine and imidazole. When the nucleobase is a pyrimidine the dN may be referred to as a “pyrimidine dN”. Exemplary pyrimidine dNs include deoxycytidine, thymidine, and deoxyuridine. When the nucleobase is a purine the dN may be referred to as a “purine dN”. Exemplary purine dNs include deoxyadenosine and deoxyguanosine.

Suitably, the deoxyribonucleosides used in the present invention are exogenous deoxyribonucleosides. An exogenous deoxyribonucleoside is one which originates outside the population of cells.

The present invention encompasses the use of deoxyribonucleoside derivatives. Deoxyribonucleoside derivatives are well known in the art and can be prepared by any suitable method. For example, WO1989/003838 and WO2005/049633 describe acyl derivatives of 2′-deoxyribonucleosides. In one embodiment, the deoxyribonucleoside derivatives are acyl derivatives (i.e. acyl deoxyribonucleosides). The dN derivatives of the present invention may increase the efficiency of transduction of an isolated population of cells by a viral vector and/or increase the efficiency of gene editing of an isolated population of cells when transduced by a viral vector. dN derivatives of the present invention may have been developed for increased solubility, increased stability and/or reduced toxicity. dN derivatives of the invention are preferably of low toxicity for mammals, in particular humans. Preferably, dN derivatives of the invention are of low toxicity for haematopoietic stem and/or progenitor cells; and/or PBMCs (e.g. T cells). Suitable dN derivatives may be identified using methods known in the art for determining transduction efficiency and/or gene editing. For example, methods for determining the percentage of cells that are transduced by a vector, or methods for determining the vector copy number per cell may be employed. Such methods are described below. The method employed is preferably one which is amenable to automation and/or high throughput screening of candidate dN derivatives. The candidate dN derivatives may form part of a library of dN derivatives.

The dN or derivative thereof may, for example, comprise or consist of deoxycytidine (dC) or a derivative thereof. The dN or derivative thereof may, for example, comprise or consist of thymidine (dT) or a derivative thereof. The dN or derivative thereof may, for example, comprise or consist of deoxyadenosine (dA) or a derivative thereof. The dN or derivative thereof may, for example, comprise or consist of deoxyguanosine (dG) or a derivative thereof.

In one embodiment, the at least one dN or derivative thereof comprises or consists of at least one pyrimidine dN or a derivative thereof. In one embodiment, the at least one dN or derivative thereof comprises deoxycytidine (dC) or a derivative thereof. In one embodiment, the at least one dN or derivative thereof comprises thymidine (dT) or a derivative thereof. In a preferred embodiment, the at least one dN or derivative thereof comprises or consists of deoxycytidine (dC) or a derivative thereof and/or thymidine (dT) or a derivative thereof. In a more preferred embodiment, the at least one dN or derivative thereof comprises or consists of dC or a derivative thereof.

The at least one dN or derivative thereof may further comprise at least one purine dN or a derivative thereof. In one embodiment, the at least one dN or derivative thereof further comprises deoxyadenosine (dA) or a derivative thereof and/or deoxyguanosine (dG) or a derivative thereof. In one embodiment, the at least one dN or derivative thereof further comprises deoxyadenosine (dA) or a derivative thereof. In one embodiment, the at least one dN or derivative thereof further comprises deoxyguanosine (dG) or a derivative thereof.

In one embodiment, the at least one dN or derivative thereof consists of one dN or a derivative thereof. In one embodiment, the at least one dN or derivative thereof consists of a dN mix. Suitably, the at least one dN or derivative thereof consists of a mix of two or more (e.g. 2, 3, 4) dNs or derivatives thereof, three or more (e.g. 3, 4) dNs or derivatives thereof, or four or more dNs or derivatives thereof. In one embodiment, the at least one dN or derivative thereof consists of two dNs or derivatives thereof. In one embodiment, the at least one dN or derivative thereof consists of three dNs or derivatives thereof. In one embodiment, the at least one dN or derivative thereof consists of four dNs or derivatives thereof. In one embodiment, the at least one dN or derivative thereof consists of dC or a derivative thereof, dT or a derivative thereof, dA or a derivative thereof, and dG or a derivative thereof.

The concentration at which the dN or a derivative thereof is present in the combination may be adjusted for different vector systems to optimise transduction efficiency and/or gene editing. Methods for determining transduction efficiency and gene editing are described below. A skilled person may therefore select a suitable concentration of the dN or a derivative thereof to maximise increase in transduction efficiency and/or gene editing while minimising any toxicity using the approaches described herein.

When the at least one dN or derivative thereof consists of a dN mix, each dN or a derivative thereof may be at the same concentration or at different concentrations. In one embodiment, each dN or a derivative thereof is at the same concentration.

In one embodiment, at least one dN or a derivative thereof is at a concentration of at least about 1 μM, at least about 5 μM, at least about 10 μM, at least about 25 μM, at least about 50 μM, or at least about 100 μM. In one embodiment, at least one dN or a derivative thereof is at a concentration of at least 25 μM. In one embodiment, at least one dN or a derivative thereof is at a concentration of at least 100 μM.

In one embodiment, at least one dN or a derivative thereof is at a concentration of 10 mM or less, 5 mM or less, 1 mM or less, or 500 μM or less. In one embodiment, at least one dN or a derivative thereof is at a concentration of 1000 μM or less. In one embodiment, at least one dN or a derivative thereof is at a concentration of 500 μM or less.

In one embodiment, at least one dN or a derivative thereof is at a concentration of from about 25 μM to about 1000 μM, from about 100 μM to about 1000 μM, or from about 100 μM to about 500 μM. In one embodiment, at least one dN or a derivative thereof is at a concentration of from about 500 μM to about 1000 μM.

In one embodiment, each dN or a derivative thereof is at a concentration of at least about 1 μM, at least about 5 μM, at least about 10 μM, at least about 25 μM, at least about 50 μM, or at least about 100 μM. In one embodiment, each dN or a derivative thereof is at a concentration of at least 25 μM. In one embodiment, each dN or a derivative thereof is at a concentration of at least 100 μM.

In one embodiment, each dN or a derivative thereof is at a concentration of 10 mM or less, 5 mM or less, 1 mM or less, or 500 μM or less. In one embodiment, each dN or a derivative thereof is at a concentration of 1000 μM or less. In one embodiment, each dN or a derivative thereof is at a concentration of 500 μM or less.

In one embodiment, each dN or a derivative thereof is at a concentration of from about 25 μM to about 1000 μM, from about 100 μM to about 1000 μM, or from about 100 μM to about 500 μM. In one embodiment, each dN or a derivative thereof is at a concentration of from about 500 μM to about 1000 μM.

### Deoxycytidine

In one aspect, the present invention provides a combination of deoxycytidine (dC) or a derivative thereof and at least one additional transduction enhancer (e.g. CsH or a derivative thereof).

Deoxycytidine (dG, CAS No. 951-77-9) is a pyrimidine deoxyribonucleoside containing cytosine and having the following structure:

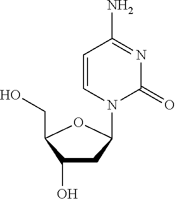 [SMILES: Nc1ccn([C@H]2C[C@@H](O)[C@@H](CO)O2)c(=O)n1]

In one embodiment, the dC or a derivative thereof is at a concentration of at least about 1 μM, at least about 5 μM, at least about 10 μM, at least about 25 μM, at least about 50 μM, or at least about 100 μM. In one embodiment, the dC or a derivative thereof is at a concentration of at least 25 μM. In one embodiment, the dC or a derivative thereof is at a concentration of at least 100 μM.

In one embodiment, the dC or a derivative thereof is at a concentration of 10 mM or less, 5 mM or less, 1 mM or less, or 500 μM or less. In one embodiment, the dC or a derivative thereof is at a concentration of 1000 μM or less. In one embodiment, the dC or a derivative thereof is at a concentration of 500 μM or less.

In one embodiment, the dC or a derivative thereof is at a concentration of from about 25 μM to about 1000 μM, from about 100 μM to about 1000 μM, or from about 100 μM to about 500 μM. In one embodiment, the dC or a derivative thereof is at a concentration of from about 500 μM to about 1000 μM. In one embodiment, the dC or a derivative thereof is at a concentration of about 500 μM.

### Thymidine

In one aspect, the present invention provides a combination of thymidine (dT) or a derivative thereof and at least one additional transduction enhancer (e.g. CsH or a derivative thereof).

Thymidine, also known as deoxythymidine, (dT, CAS No. 50-89-5) is a pyrimidine deoxyribonucleoside containing thymine and having the following structure:

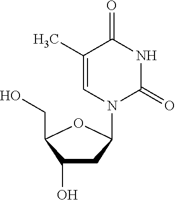 [SMILES: Cc1cn([C@H]2C[C@@H](O)[C@@H](CO)O2)c(=O)[nH]c1=O]

In one embodiment, the dT or a derivative thereof is at a concentration of at least about 1 μM, at least about 5 μM, at least about 10 μM, at least about 25 μM, at least about 50 μM, or at least about 100 μM. In one embodiment, the dT or a derivative thereof is at a concentration of at least 25 μM. In one embodiment, the dT or a derivative thereof is at a concentration of at least 100 μM.

In one embodiment, the dT or a derivative thereof is at a concentration of 10 mM or less, 5 mM or less, 1 mM or less, or 500 μM or less. In one embodiment, the dT or a derivative thereof is at a concentration of 1000 μM or less. In one embodiment, the dT or a derivative thereof is at a concentration of 500 μM or less.

In one embodiment, the dT or a derivative thereof is at a concentration of from about 25 μM to about 1000 μM, from about 100 μM to about 1000 μM, or from about 100 μM to about 500 μM. In one embodiment, the dT or a derivative thereof is at a concentration of from about 500 μM to about 1000 μM. In one embodiment, the dT or a derivative thereof is at a concentration of about 500 μM.

### Deoxyadenosine

In one aspect, the present invention provides a combination of deoxyadenosine (dA) or a derivative thereof and at least one additional transduction enhancer (e.g. CsH or a derivative thereof).

Deoxyadenosine (dA, CAS No. 958-09-8) is a purine deoxyribonucleoside containing adenine and having the following structure:

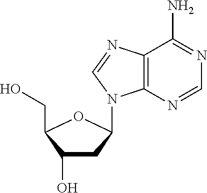 [SMILES: Nc1ncnc2c1ncn2[C@H]1C[C@@H](O)[C@@H](CO)O1]

In one embodiment, the dA or a derivative thereof is at a concentration of at least about 1 μM, at least about 5 μM, at least about 10 μM, at least about 25 μM, at least about 50 μM, or at least about 100 μM. In one embodiment, the dA or a derivative thereof is at a concentration of at least 25 μM. In one embodiment, the dA or a derivative thereof is at a concentration of at least 100 μM.

In one embodiment, the dA or a derivative thereof is at a concentration of 10 mM or less, 5 mM or less, 1 mM or less, or 500 μM or less. In one embodiment, the dA or a derivative thereof is at a concentration of 1000 μM or less. In one embodiment, the dA or a derivative thereof is at a concentration of 500 μM or less.

In one embodiment, the dA or a derivative thereof is at a concentration of from about 25 μM to about 1000 μM, from about 100 μM to about 1000 μM, or from about 100 μM to about 500 μM. In one embodiment, the dA or a derivative thereof is at a concentration of from about 500 μM to about 1000 μM. In one embodiment, the dA or a derivative thereof is at a concentration of about 500 μM.

### Deoxyguanosine

In one aspect, the present invention provides a combination of deoxyguanosine (dG) or a derivative thereof and at least one additional transduction enhancer (e.g. CsH or a derivative thereof).

Deoxyguanosine (dG, CAS No. 961-07-9) is a purine deoxyribonucleoside containing guanine and having the following structure:

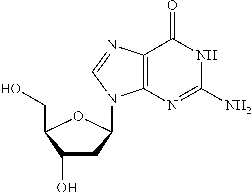 [SMILES: Nc1nc2c(ncn2[C@H]2C[C@@H](O)[C@@H](CO)O2)c(=O)[nH]1]

In one embodiment, the dG or a derivative thereof is at a concentration of at least about 1 μM, at least about 5 μM, at least about 10 μM, at least about 25 μM, at least about 50 μM, or at least about 100 μM. In one embodiment, the dG or a derivative thereof is at a concentration of at least 25 μM. In one embodiment, the dG or a derivative thereof is at a concentration of at least 100 μM.

In one embodiment, the dG or a derivative thereof is at a concentration of 10 mM or less, 5 mM or less, 1 mM or less, or 500 μM or less. In one embodiment, the dG or a derivative thereof is at a concentration of 1000 μM or less. In one embodiment, the dG or a derivative thereof is at a concentration of 500 μM or less.

In one embodiment, the dG or a derivative thereof is at a concentration of from about 25 μM to about 1000 μM, from about 100 μM to about 1000 μM, or from about 100 μM to about 500 μM. In one embodiment, the dG or a derivative thereof is at a concentration of from about 500 μM to about 1000 μM. In one embodiment, the dG or a derivative thereof is at a concentration of about 500 μM.

### Pyrimidine Precursors

The present invention encompasses the use of pyrimidine precursors. As used herein, the term “pyrimidine precursor” may refer to a compound that is upstream of the pyrimidine in the natural biosynthetic pathway of the pyrimidine. Preferably, the pyrimidine is a pyrimidine dNTP. Preferably, the pyrimidine precursor is a pyrimidine dNTP precursor. For example, the pyrimidine precursor may be an intermediate in the biosynthetic pathway between orotic acid (OA) and the pyrimidine dNTP. Preferably, the pyrimidine precursor is not a dNTP.

The pyrimidine precursor may be, for example, orotic acid (OA), orotidine 5′-monophosphate (OMP), uridine 5′-monophosphate (UMP), uridine 5′-diphosphate (UDP) or uridine 5′-triphosphate (UTP).

In one embodiment, the at least one pyrimidine precursor comprises or consists of orotic acid (OA). In one aspect, the present invention provides a combination of orotic acid (OA) and at least one additional transduction enhancer (e.g. CsH or a derivative thereof).

In one embodiment, the OA is at a concentration of from about 1 μM to about 50 μM. In one embodiment, the OA is at a concentration of from about 1 μM to about 25 μM. In one embodiment, the OA is at a concentration of from about 1 μM to about 15 μM. In one embodiment, the OA is at a concentration of from about 1 μM to about 10 μM. In one embodiment, the OA is at a concentration of from about 5 μM to about 50 μM. In one embodiment, the OA is at a concentration of from about 5 μM to about 25 μM. In one embodiment, the OA is at a concentration of from about 5 μM to about 15 μM. In one embodiment, the OA is at a concentration of from about 5 μM to about 10 μM. In one embodiment, the OA is at a concentration of about 7.5 μM.

In one embodiment, the at least one pyrimidine precursor comprises or consists of uridine 5′-monophosphate (UMP). In one aspect, the present invention provides a combination of uridine 5′-monophosphate (UMP) and at least one additional transduction enhancer (e.g. CsH or a derivative thereof).

In one embodiment, the UMP is at a concentration of from about 0.1 to about 10 mM. In one embodiment, the UMP is at a concentration of from about 0.5 to about 10 mM. In one embodiment, the UMP is at a concentration of from about 0.1 to about 5 mM. In one embodiment, the UMP is at a concentration of from about 0.5 to about 5 mM. In one embodiment, the UMP is at a concentration of from about 0.1 to about 2.5 mM. In one embodiment, the UMP is at a concentration of from about 0.5 to about 2.5 mM. In one embodiment, the UMP is at a concentration of from about 0.1 to about 1.5 mM. In one embodiment, the UMP is at a concentration of from about 0.5 to about 1.5 mM. In one embodiment, the UMP is at a concentration of about 1 mM.

In one embodiment, the at least one pyrimidine precursor comprises or consists of orotidine 5′-monophosphate (OMP). In one aspect, the present invention provides a combination of orotidine 5′-monophosphate (OMP) and at least one additional transduction enhancer (e.g. CsH or a derivative thereof).

In one embodiment, the at least one pyrimidine precursor comprises or consists of uridine 5′-diphosphate (UDP). In one aspect, the present invention provides a combination of uridine 5′-diphosphate (UDP) and at least one additional transduction enhancer (e.g. CsH or a derivative thereof).

In one embodiment, the at least one pyrimidine precursor comprises or consists of uridine 5′-triphosphate (UTP). In one aspect, the present invention provides a combination of uridine 5′-triphosphate (UTP) and at least one additional transduction enhancer (e.g. CsH or a derivative thereof).

### Cyclosporin H

In preferred embodiments, the at least one additional transduction enhancer comprises or consists of cyclosporin H (CsH) or a derivative thereof.

The present invention provides a combination of at least one deoxyribonucleoside (dN) or a derivative thereof and cyclosporin H (CsH) or a derivative thereof. In one aspect, the invention provides a combination of at least one pyrimidine precursor and cyclosporin H (CsH) or a derivative thereof. In one aspect, the present invention provides a combination of at least one pyrimidine deoxyribonucleoside (dN) or a derivative thereof and cyclosporin H (CsH) or a derivative thereof. In one aspect, the present invention provides a combination of at least one pyrimidine pyrimidine precursor and cyclosporin H (CsH) or a derivative thereof. In one aspect, the present invention provides a combination of deoxycytidine (dC) or a derivative thereof and cyclosporin H (CsH) or a derivative thereof. In one aspect, the present invention provides a combination of thymidine (dT) or a derivative thereof and cyclosporin H (CsH) or a derivative thereof. In one aspect, the present invention provides a combination of deoxyadenosine (dA) or a derivative thereof and cyclosporin H (CsH) or a derivative thereof. In one aspect, the present invention provides a combination of deoxyguanosine (dG) or a derivative thereof and cyclosporin H (CsH) or a derivative thereof. In one aspect, the present invention provides a combination of orotic acid (OA) and cyclosporin H (CsH) or a derivative thereof. In one aspect, the present invention provides a combination of uridine 5′-monophosphate (UMP) and cyclosporin H (CsH) or a derivative thereof.

Cyclosporin H (CsH, CAS No. 83602-39-5) is a cyclic undecapeptide having the following structure:

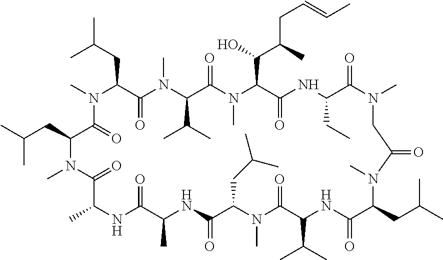

CsH is known to selectively antagonise the formyl peptide receptor, however unlike cyclosporin A (CsA), CsH does not bind cyclophilin to evoke immunosuppression. CsA mediates immunosuppression as a complex with the host peptidyl-prolyl isomerase cyclophilin A (CypA). This inhibits the Ca²⁺-dependent phosphatase calcineurin and consequent activation of pro-inflammatory cytokines such as IL-2 (Sokolskaja, E. et al. (2006) Curr. Opin. Microbiol. 9: 404-8).

The present invention encompasses the use of CsH and derivatives of CsH. The CsH derivatives of the present invention may increase the efficiency of transduction of an isolated population of cells by a viral vector and/or increase the efficiency of gene editing of an isolated population of cells when transduced by a viral vector. CsH derivatives of the present invention may have been developed for increased solubility, increased stability and/or reduced toxicity. CsH derivatives of the invention are preferably of low toxicity for mammals, in particular humans. Preferably, CsH derivatives of the invention are of low toxicity for haematopoietic stem and/or progenitor cells; and/or PBMCs (e.g. T cells).

Suitable CsH derivatives may be identified using methods known in the art for determining transduction efficiency and/or gene editing. For example, methods for determining the percentage of cells that are transduced by a vector, or methods for determining the vector copy number per cell may be employed. Such methods are described below. The method employed is preferably one which is amenable to automation and/or high throughput screening of candidate CsH derivatives. The candidate CsH derivatives may form part of a library of CsH derivatives.

The concentration at which CsH or a derivative thereof is present may be adjusted for different vector systems to optimise transduction efficiency and/or gene editing. Methods for determining transduction efficiency and gene editing are described below. A skilled person may therefore select a suitable concentration of CsH or a derivative thereof to maximise increase in transduction efficiency and/or gene editing while minimising any toxicity using the approaches described herein.

In one embodiment, the CsH or derivative thereof is at a concentration of about 1-50 μM. In another embodiment, the CsH or derivative thereof is at a concentration of about 5-50 μM. In another embodiment, the CsH or derivative thereof is at a concentration of about 10-50 μM.

In another embodiment, the CsH or derivative thereof is at a concentration of about 1-40, 5-40 or 10-40 μM. In another embodiment, the CsH or derivative thereof is at a concentration of about 1-30, 5-30 or 10-30 μM. In another embodiment, the CsH or derivative thereof is at a concentration of about 1-20, 5-20 or 10-20 μM. In another embodiment, the CsH or derivative thereof is at a concentration of about 1-15, 5-15 or 10-15 μM.

In another embodiment, the CsH or derivative thereof is at a concentration of about 1-15, 2-14, 3-13, 4-12, 5-11, 6-10 or 7-9 μM. For example, the concentration of CsH may be about 1, 2, 3, 4, 5, 6, 7, 8, 9, 10, 11, 12, 13, 14, 15, 20, 25, 30, 35, 40, 45 or 50 μM. In a preferred embodiment, the concentration of CsH or a derivative thereof is about 5, 6, 7, 8, 9, 10, 11, 12, 13, 14 or 15 μM. In a particularly preferred embodiment, the concentration of CsH or a derivative thereof is about 10 μM.

A combination of at least one dN or a derivative thereof and CsH or a derivative thereof for use in the present invention may be prepared using routine methods known in the art. The ratio at which the at least one dN or a derivative thereof CsH or a derivative thereof is present may be adjusted for different vector systems to optimise transduction efficiency and/or gene editing. The ratio at which the at least one pyrimidine precursor and CsH or a derivative thereof is present may be adjusted for different vector systems to optimise transduction efficiency and/or gene editing. Methods for determining transduction efficiency and gene editing are described below. A skilled person may therefore select a suitable ratio to maximise increase in transduction efficiency and/or gene editing while minimising any toxicity using the approaches described herein.

When a dN mix is applied, each dN or a derivative thereof may be at the same ratio or at different ratios. In one embodiment, each dN or a derivative thereof is at the same ratio.

In one embodiment, at least one dN or a derivative thereof (or each dN or a derivative thereof if a dN mix is used) and CsH or a derivative thereof are at a molar ratio (dN:CsH) of at least about 0.5, at least about 1, at least about 2, at least about 3, at least about 5, or at least about 10. In one embodiment, at least one dN or a derivative thereof and CsH or a derivative thereof are at a molar ratio (dN:CsH) of about 1000 or less, about 500 or less, about 200 or less, or about 100 or less. In one embodiment, at least one dN or a derivative thereof and CsH or a derivative thereof are at a molar ratio (dN:CsH) of from about 0.5 to about 1000, from about 1 to about 1000, from about 2 to about 200, from about 2 to about 100, from about 5 to about 100, or from about 10 to about 100. In one embodiment, at least one dN or a derivative thereof and CsH or a derivative thereof are at a molar ratio (dN:CsH) of from about 25:8 to about 1000:8 or from about 100:8 to about 500:8. In one embodiment, at least one dN or a derivative thereof and CsH or a derivative thereof are at a molar ratio (dN:CsH) of about 50.

In one embodiment, each dN or a derivative thereof (or each dN or a derivative thereof if a dN mix is used) and CsH or a derivative thereof are at a molar ratio (dN:CsH) of at least about 0.5, at least about 1, at least about 2, at least about 3, at least about 5, or at least about 10. In one embodiment, each dN or a derivative thereof and CsH or a derivative thereof are at a molar ratio (dN:CsH) of about 1000 or less, about 500 or less, about 200 or less, or about 100 or less. In one embodiment, each dN or a derivative thereof and CsH or a derivative thereof are at a molar ratio (dN:CsH) of from about 0.5 to about 1000, from about 1 to about 1000, from about 2 to about 200, from about 2 to about 100, from about 5 to about 100, or from about 10 to about 100. In one embodiment, each dN or a derivative thereof and CsH or a derivative thereof are at a molar ratio (dN:CsH) of from about 25:8 to about 1000:8 or from about 100:8 to about 500:8. In one embodiment, each dN or a derivative thereof and CsH or a derivative thereof are at a molar ratio (dN:CsH) of about 50.

In one embodiment, at least one pyrimidine dN or a derivative thereof and CsH or a derivative thereof are at a molar ratio (dN:CsH) of at least about 0.5, at least about 1, at least about 2, at least about 3, at least about 5, or at least about 10. In one embodiment, at least one pyrimidine dN or a derivative thereof and CsH or a derivative thereof are at a molar ratio (dN:CsH) of about 1000 or less, about 500 or less, about 200 or less, or about 100 or less. In one embodiment, at least one pyrimidine dN or a derivative thereof and CsH or a derivative thereof are at a molar ratio (dN:CsH) of from about 0.5 to about 1000, from about 1 to about 1000, from about 2 to about 200, from about 2 to about 100, from about 5 to about 100, or from about 10 to about 100. In one embodiment, the pyrimidine dN or a derivative thereof and CsH or a derivative thereof are at a molar ratio (dN:CsH) of from about 25:8 to about 1000:8 or from about 100:8 to about 500:8. In one embodiment, the pyrimidine dN or a derivative thereof and CsH or a derivative thereof are at a molar ratio (dN:CsH) of about 50.

In one embodiment, dC or a derivative thereof and CsH or a derivative thereof are at a molar ratio (dC:CsH) of at least about 0.5, at least about 1, at least about 2, at least about 3, at least about 5, or at least about 10. In one embodiment, dC or a derivative thereof and CsH or a derivative thereof are at a molar ratio (dC:CsH) of about 1000 or less, about 500 or less, about 200 or less, or about 100 or less. In one embodiment, dC or a derivative thereof and CsH or a derivative thereof are at a molar ratio (dC:CsH) of from about 0.5 to about 1000, from about 1 to about 1000, from about 2 to about 200, from about 2 to about 100, from about 5 to about 100, or from about 10 to about 100. In one embodiment, the dC or a derivative thereof and CsH or a derivative thereof are at a molar ratio (dC:CsH) of from about 25:8 to about 1000:8 or from about 100:8 to about 500:8. In one embodiment, the dC or a derivative thereof and CsH or a derivative thereof are at a molar ratio (dC:CsH) of about 50.

In one embodiment, dT or a derivative thereof and CsH or a derivative thereof are at a molar ratio (dT:CsH) of at least about 0.5, at least about 1, at least about 2, at least about 3, at least about 5, or at least about 10. In one embodiment, dT or a derivative thereof and CsH or a derivative thereof are at a molar ratio (dT:CsH) of about 1000 or less, about 500 or less, about 200 or less, or about 100 or less. In one embodiment, dT or a derivative thereof and CsH or a derivative thereof are at a molar ratio (dT:CsH) of from about 0.5 to about 1000, from about 1 to about 1000, from about 2 to about 200, from about 2 to about 100, from about 5 to about 100, or from about 10 to about 100. In one embodiment, the dT or a derivative thereof and CsH or a derivative thereof are at a molar ratio (dT:CsH) of from about 25:8 to about 1000:8 or from about 100:8 to about 500:8. In one embodiment, the dT or a derivative thereof and CsH or a derivative thereof are at a molar ratio (dT:CsH) of about 50.

In one embodiment, dA or a derivative thereof and/or dG or a derivative thereof and CsH or a derivative thereof are at a molar ratio (dN:CsH) of at least about 0.5, at least about 1, at least about 2, at least about 3, at least about 5, or at least about 10. In one embodiment, dA or a derivative thereof and/or dG or a derivative thereof and CsH or a derivative thereof are at a molar ratio (dN:CsH) of about 1000 or less, about 500 or less, about 200 or less, or about 100 or less. In one embodiment, dA or a derivative thereof and/or dG or a derivative thereof and CsH or a derivative thereof are at a molar ratio (dN:CsH) of from about 0.5 to about 1000, from about 1 to about 1000, from about 2 to about 200, from about 2 to about 100, from about 5 to about 100, or from about 10 to about 100. In one embodiment, the dA or a derivative thereof and/or dG or a derivative thereof and CsH or a derivative thereof are at a molar ratio (dN:CsH) of from about 25:8 to about 1000:8 or from about 100:8 to about 500:8. In one embodiment, the dA or a derivative thereof and/or dG or a derivative thereof and CsH or a derivative thereof are at a molar ratio (dN:CsH) of about 50.

### Other Transduction Enhancers

The dN or a derivative thereof (and optionally the CsH or a derivative thereof) may be in combination with any other additional transduction enhancer. The pyrimidine precursor (and optionally the CsH or a derivative thereof) may be in combination with any other additional transduction enhancer.

As used herein, a “transduction enhancer” may refer to any agent capable of increasing the efficiency of transduction. Exemplary transduction enhancers include enhancers of prostaglandin EP receptor signalling (e.g. PGE2, dmPGE2, derivatives, analogues and precursors of PGE2); CAMP activators (e.g. cAMP/PI3K/AKT agonists); ABC transporter inhibitors (e.g. verapamil, quinidine, diltiazem, ritonavir); mTOR inhibitors (e.g. rapamycin or a derivative thereof); beta-deliverin; inhibitors of cofilin phosphorylation (e.g. Staurosporin); CsA and derivatives thereof; LentiBOOST and other poloxamers (e.g. Pluronic compounds P108 (P338), L35 (P105), L43 (P123), L44 (P124), L64 (P184), F68 (P188), P85 (P235), F98 (P288), F127 (P407), P123 (P403), P104 (P334), L101 (P331), F87, F88); poloxamine compounds (e.g. T304, T701, T901, T904, T908, T1107, T1301, T1304, T1307, 9R4, 15R1); retronectin; vectofusin-1 and derivatives thereof; polybrene; protamine sulphater; DEAE-Dextran; human semen-derived enhancer of infection; HIVgp120-derived peptides; b1 receptor blockers and selective serotonin reuptake inhibitors; Vpx; nanofibrils (e.g. EF-C peptides); epigenetic drugs; proteosomal inhibitors; kinase and kinase receptor inhibtors; central DNA flap/PPT; and cationic lipids or recombinant fibronectin.

In one embodiment, the at least one additional transduction enhancer (optionally, in addition to CsH) is selected from: an enhancer of prostaglandin EP receptor signalling (e.g. PGE2, dmPGE2, derivatives, analogues and precursors of PGE2); a CAMP activator (e.g. CAMP/PI3K/AKT agonists); an ABC transporter inhibitor (e.g. verapamil, quinidine, diltiazem, ritonavir); a mTOR inhibitor (e.g. rapamycin or a derivative thereof); beta-deliverin; an inhibitor of cofilin phosphorylation (e.g. Staurosporin); CsA or a derivative thereof; LentiBOOST or other poloxamer (e.g. Pluronic compounds P108 (P338), L35 (P105), L43 (P123), L44 (P124), L64 (P184), F68 (P188), P85 (P235), F98 (P288), F127 (P407), P123 (P403), P104 (P334), L101 (P331), F87, F88); a poloxamine compound (e.g. T304, T701, T901, T904, T908, T1107, T1301, T1304, T1307, 9R4, 15R1); retronectin; vectofusin-1 or a derivative thereof; polybrene; protamine sulphate; DEAE-Dextran; human semen-derived enhancer of viral infection (SEVI); a HIV gp120-derived peptide; a b1 receptor blocker or a selective serotonin reuptake inhibitor; Vpx; a nanofibril (e.g. an EF-C peptide); an epigenetic drug; a proteosome inhibitor; a kinase or kinase receptor inhibitor; a central DNA flap or PPT; a cationic lipid or a recombinant fibronectin.

In one embodiment, the at least one additional transduction enhancer (optionally, in addition to CsH) comprises an enhancer of prostaglandin EP receptor signalling (e.g. PGE2, dmPGE2, derivatives, analogues and precursors of PGE2). Prostaglandin E2 (PGE2), which is also known as dinoprostone, is a naturally occurring prostaglandin having the structure:

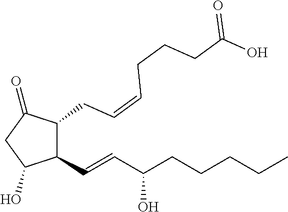 [SMILES: CCCCC[C@H](O)/C=C/[C@H]1[C@H](O)CC(=O)[C@@H]1C/C=C\CCCC(=O)O]

Prostaglandin E2 or a prostaglandin E2 derivative may be used, according to the invention in combination with a dN or a derivative thereof (and optionally CsH or a derivative thereof) for increasing transduction efficiency and/or gene editing efficiency of an isolated population of cells. In one embodiment, the prostaglandin E2 derivative is 16,16-dimethyl prostaglandin E2. By derivative of prostaglandin E2, it is to be understood that prostaglandin E2 is modified by any of a number of techniques known in the art, preferably to improve properties such as stability and activity, while still retaining its function of increasing transduction efficiency and/or gene editing efficiency of an isolated population of cells. WO2007112084, for example, describes agents that stimulate the PGE2 pathway.

In one embodiment, the at least one additional transduction enhancer (optionally, in addition to CsH) comprises a cAMP activator (e.g. cAMP/PI3K/AKT agonists). WO2013049615, for example, describes compounds that stimulate the prostaglandin EP receptor signaling pathway by increasing signaling through the cAMP/P13K/AKT second messenger pathway.

In one embodiment, the at least one additional transduction enhancer (optionally, in addition to CsH) comprises an ABC transporter inhibitor (e.g. verapamil, quinidine, diltiazem, ritonavir). WO2004098531, for example, describes increased transduction using ABC transporter inhibitors.

In one embodiment, the at least one additional transduction enhancer (optionally, in addition to CsH) comprises a mTOR inhibitor (e.g. rapamycin or a derivative thereof). Rapamycin (CAS No. 53123-88-9, also known as Sirolimus) is a macrolide produced by Streptomyces hygroscopicus. Rapamycin has the following structure:

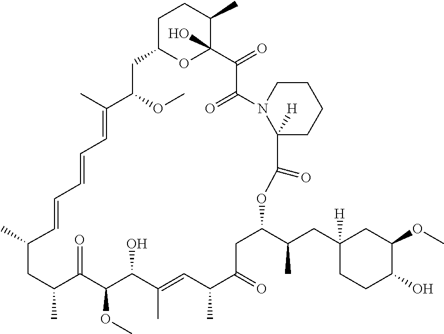

Rapamycin is an approved immunosuppressive agent for use in prevention of allograft rejection. By derivative of rapamycin, it is to be understood that rapamycin is modified by any of a number of techniques known in the art, preferably to improve properties such as stability and activity, while still retaining its function of increasing transduction efficiency and/or gene editing efficiency of an isolated population of cells.

In one embodiment, the at least one additional transduction enhancer (optionally, in addition to CsH) comprises beta-deliverin.

In one embodiment, the at least one additional transduction enhancer (optionally, in addition to CsH) comprises an inhibitor of cofilin phosphorylation (e.g. Staurosporin). Staurosporine is a natural product originally isolated from Streptomyces staurosporeus. It displays activity as an inhibitor of protein kinases through the prevention of ATP binding to the kinase.

In one embodiment, the at least one additional transduction enhancer (optionally, in addition to CsH) comprises CsA or a derivative thereof. Petrillo C, et al (2015) Mol Ther 23: 352-362 describes that cyclosporin A (CsA) and rapamycin relieve distinct lentiviral restriction blocks in hematopoietic stem and progenitor cells.

In one embodiment, the at least one additional transduction enhancer (optionally, in addition to CsH) comprises LentiBOOST or another poloxamer (e.g. Pluronic compounds P108 (P338), L35 (P105), L43 (P123), L44 (P124), L64 (P184), F68 (P188), P85 (P235), F98 (P288), F127 (P407), P123 (P403), P104 (P334), L101 (P331), F87, F88). WO2013127964, for example, describes retroviral transduction using poloxamers.

In one embodiment, the at least one additional transduction enhancer (optionally, in addition to CsH) comprises a poloxamine compound (e.g. T304, T701, T901, T904, T908, T1107, T1301, T1304, T1307, 9R4, 15R1). WO2003066104, WO2020115114, WO2020247814 and WO2021076993, for example, describes using poloxamines to improve in vivo gene transfer.

In one embodiment, the at least one additional transduction enhancer (optionally, in addition to CsH) comprises Boost A.

In one embodiment, the at least one additional transduction enhancer (optionally, in addition to CsH) comprises retronectin.

In one embodiment, the at least one additional transduction enhancer (optionally, in addition to CsH) comprises vectofusin-1 or a derivative thereof.

In one embodiment, the at least one additional transduction enhancer (optionally, in addition to CsH) comprises polybrene.

In one embodiment, the at least one additional transduction enhancer (optionally, in addition to CsH) comprises protamine sulphate.

In one embodiment, the at least one additional transduction enhancer (optionally, in addition to CsH) comprises DEAE-Dextran.

In one embodiment, the at least one additional transduction enhancer (optionally, in addition to CsH) comprises human semen-derived enhancer of viral infection (SEVI).

In one embodiment, the at least one additional transduction enhancer (optionally, in addition to CsH) comprises a HIV gp120-derived peptide.

In one embodiment, the at least one additional transduction enhancer (optionally, in addition to CsH) comprises a b1 receptor blocker or a selective serotonin reuptake inhibitor.

In one embodiment, the at least one additional transduction enhancer (optionally, in addition to CsH) comprises Vpx.

In one embodiment, the at least one additional transduction enhancer (optionally, in addition to CsH) comprises a nanofibril (e.g. an EF-C peptide).

In one embodiment, the at least one additional transduction enhancer (optionally, in addition to CsH) comprises an epigenetic drug.

In one embodiment, the at least one additional transduction enhancer (optionally, in addition to CsH) comprises a proteosome inhibitor.

In one embodiment, the at least one additional transduction enhancer (optionally, in addition to CsH) comprises a kinase or kinase receptor inhibitor. I

In one embodiment the at least one additional transduction enhancer (optionally, in addition to CsH) comprises a central DNA flap or PPT.

In one embodiment, the at least one additional transduction enhancer (optionally, in addition to CsH) comprises a cationic lipid or a recombinant fibronectin.

The dN or a derivative thereof (and optionally the CsH or a derivative thereof) may be in combination with any other suitable agents. The pyrimidine precursor (and optionally the CsH or a derivative thereof) may be in combination with any other suitable agents. In one embodiment, the combination of the invention further comprises one or more additional agents. The one or more additional agents may include, for example, one or more cell culture supplement, including antibiotics (e.g. penicillin, streptomycin), amino acids (e.g. glutamine), carbohydrates (e.g. glucose, galactose, maltose, fructose, pyruvate), vitamins (e.g. vitamin B12, vitamin A, vitamin E, riboflavin, thiamine, biotin), inorganic salts (e.g. sodium salts, potassium salts, calcium salts), buffers (e.g. HEPES), proteins (e.g. albumin, transferrin, fibronectin, fetuin, growth factors), lipids and fatty acids (e.g. cholesterol, steroids), and trace elements (e.g. zinc, copper, selenium).

### Increasing the Efficiency of Transduction and Gene Editing

### Increasing the Efficiency of Transduction

Increasing the efficiency of transduction refers to an increase in the transduction of the cells (e.g. haematopoietic stem and/or progenitor cells; or PMBCs (e.g. T cells)) in the presence of an agent (e.g. a dN or a derivative thereof), in comparison to the transduction achieved in the absence of the agent but under otherwise substantially identical conditions. An increased efficiency of transduction may therefore allow the multiplicity of infection (MOI) and/or the transduction time required to achieve effective transduction to be reduced.

In one embodiment, the percentage of cells transduced by the vector is increased. In another embodiment, the vector copy number per cell is increased. Preferably, both are achieved at the same time.

Methods for determining the percentage of cells transduced by a vector are known in the art. Suitable methods include flow cytometry, fluorescence-activated cell sorting (FACS) and fluorescence microscopy. The technique employed is preferably one which is amenable to automation and/or high throughput screening.

For example, a population of cells may be transduced with a vector which harbours a reporter gene. The vector may be constructed such that the reporter gene is expressed when the vector transduces a cell. Suitable reporter genes include genes encoding fluorescent proteins, for example green, yellow, cherry, cyan or orange fluorescent proteins. Once the population of cells has been transduced by the vector, both the number of cells expressing and not-expressing the reporter gene may be quantified using a suitable technique, such as FACS. The percentage of cells transduced by the vector may then be calculated.

Alternatively, quantitative PCR (qPCR) may be used to determine the percentage of cells transduced by a vector that does not harbour a reporter gene. For example, single colonies of cells (e.g. CD34+ cells) may be picked from a semi-solid culture and qPCR may be performed on each colony separately to determine the percentage of vector-positive colonies among those analysed.

Methods for determining vector copy number are also known in the art. The technique employed is preferably one which is amenable to automation and/or high throughput screening. Suitable techniques include quantitative PCR (qPCR) and Southern blot-based approaches.

### Increasing the Efficiency of Gene Editing

Increasing the efficiency of gene editing may refer to an increase in the number of cells (e.g. haematopoietic stem and/or progenitor cells; or PBMCs (e.g. T cells)) in which a target gene or site has been edited (e.g. disrupted, replaced, deleted or had a nucleic acid sequence inserted within or at it) in the intended manner following transduction of a population of cells with a viral vector in the presence of an agent (e.g. a dN or a derivative thereof), in comparison to that achieved in the absence of the agent but under otherwise substantially identical conditions. An increased efficiency of gene editing may therefore allow the multiplicity of infection (MOI) and/or the transduction time required to achieve effective gene editing to be reduced. Methods for determining whether a target gene or site has been edited are known in the art.

In the context of gene editing, for example using a CRISPR/Cas system, preferably the vector used to transduce the population of cells is a non-integrating vector (e.g. an integration-defective lentiviral vector, IDLV).

### Compositions and Kits

### Compositions

In one aspect, the present invention provides a composition comprising the combination of the invention.

In one aspect, the present invention provides a composition comprising at least one deoxyribonucleoside (dN) or a derivative thereof and at least one additional transduction enhancer (e.g. cyclosporin H (CsH) or a derivative thereof). In one aspect, the present invention provides a composition comprising at least one pyrimidine precursor and at least one additional transduction enhancer (e.g. cyclosporin H (CsH) or a derivative thereof).

In one aspect, the present invention provides a composition comprising at least one pyrimidine dN or a derivative thereof and at least one additional transduction enhancer (e.g. cyclosporin H (CsH) or a derivative thereof).

In one aspect, the present invention provides a composition comprising dC or a derivative thereof and at least one transduction enhancer (e.g. CsH or a derivative thereof).

In one aspect, the present invention provides a composition comprising dT or a derivative thereof and at least one transduction enhancer (e.g. CsH or a derivative thereof).

In one aspect, the present invention provides a composition comprising orotic acid (OA) and at least one transduction enhancer (e.g. CsH or a derivative thereof).

In one aspect, the present invention provides a composition comprising uridine 5′-monophosphate (UMP) and at least one transduction enhancer (e.g. CsH or a derivative thereof).

The composition of the invention may be any composition suitable for increasing the efficiency of transduction of an isolated population of cells by a viral vector and/or increasing the efficiency of gene editing of an isolated population of cells when transduced by a viral vector. For example, the composition may be a cell medium or a supplement for a cell medium (e.g. a media supplement). The composition may be provided in any suitable form such as a liquid, gel, or a dry (e.g. powder) form.

In one embodiment, the composition is a cell medium. As used herein, a “cell medium” or “cell culture medium” may refer to a liquid intended to support the growth or maintenance of cells (e.g. human cells). The cells may be any cells referred to herein, e.g. HSPCs and/or PBMCs. Suitable cell media is well known in the art and may comprise essential nutrients (e.g. amino acids, carbohydrates, vitamins, minerals), growth factors, hormones, and/or buffers to support the growth or maintenance of the cells. Suitably, the cell medium is a transduction medium. As used herein a “transduction medium” may refer to any cell medium designed to support transduction of cells (e.g. human cells).

Suitably, the composition (e.g. when the composition is a cell medium) may comprise the dN or a derivative thereof (or each dN or a derivative thereof if a dN mix is used) in the concentrations described above.

In one embodiment, the composition is a media supplement. As used herein, a “media supplement”, a “cell media supplement”, or a “cell culture supplement” may refer to a liquid, gel, or powder intended to be added to a cell media to supplement it with one or more components. Suitably, the media supplement is a transduction media supplement.

Suitably, the composition (e.g. when the composition is a media supplement) may comprise the dN or a derivative thereof (or each dN or a derivative thereof if a dN mix is used) in a concentration which is concentrated more than one times compared to the concentrations described above. Suitably, the composition (e.g. when the composition is a media supplement) may comprise the pyrimidine precursor in a concentration which is concentrated more than one times compared to the concentrations described above. Suitably, the composition is concentrated at least about 2 times, at least about 5 times, at least about 10 times, at least about 50 times, or at least about 100 times. Suitably, the composition is concentrated about 1000 times or less, about 500 times or less, or about 100 times or less. Suitably, the composition is concentrated from about 10 times to about 1000 times. Suitably, the composition is concentrated from about 10 times to about 100 times. In one embodiment, the composition is concentrated about 10 times.

For example, the composition (e.g. when the composition is a media supplement) may comprise at least one dN or a derivative thereof (or each dN or a derivative thereof if a dN mix is used) at a concentration of at least about 10 μM, at least about 50 μM, at least about 100 μM, at least about 250 μM, at least about 500 μM, or at least about 1000 μM. For example, the composition (e.g. when the composition is a media supplement) may comprise at least one dN or a derivative thereof (or each dN or a derivative thereof if a dN mix is used) at a concentration of 100 mM or less, 50 mM or less, 10 mM or less, or 5000 μM or less. For example, the composition (e.g. when the composition is a media supplement) may comprise at least one dN or a derivative thereof (or each dN or a derivative thereof if a dN mix is used) at a concentration of from about 250 μM to about 10 mM, from about 1000 μM to about 10 mM, or from about 1000 μM to about 5 mM. For example, the composition (e.g. when the composition is a media supplement) may comprise at least one dN or a derivative thereof (or each dN or a derivative thereof if a dN mix is used) at a concentration of from about 5 mM to about 10 mM.

### Cyclosporin H

In preferred embodiments, the composition comprises cyclosporin H (CsH) or a derivative thereof.

Suitably, the composition (e.g. when the composition is a cell medium) may comprise CsH or a derivative thereof in the same concentration described above. Suitably, the composition (e.g. when the composition is a media supplement) may comprise the CsH or derivative thereof in a concentration which is concentrated more than described above. For example, the composition (e.g. when the composition is a media supplement) may comprise the CsH or a derivative thereof at a concentration of about 10-500 μM, about 50-500 μM, or about 100-500 μM.

### Other Agents

In one embodiment, the composition further comprises one or more additional transduction enhancers (optionally in addition to CsH or a derivative thereof).

In one embodiment, the composition of the invention further comprises one or more additional agents. For example, when the composition is a cell medium it may comprise and other suitable agents, such as serum, antibiotics (e.g. penicillin, streptomycin), amino acids (e.g. glutamine), carbohydrates (e.g. glucose, galactose, maltose, fructose, pyruvate), vitamins (e.g. vitamin B12, vitamin A, vitamin E, riboflavin, thiamine, biotin), inorganic salts (e.g. sodium salts, potassium salts, calcium salts), buffers (e.g. HEPES), proteins (e.g. albumin, transferrin, fibronectin, fetuin, growth factors), lipids and fatty acids (e.g. cholesterol, steroids), and trace elements (e.g. zince, copper, selenium).

### Kits

In one aspect, the present invention provides a kit comprising a combination or composition according to the present invention and, optionally, cell media and/or a population of cells (e.g. HSPCs and/or PBMCs).

In one aspect, the present invention provides a kit comprising at least one deoxyribonucleoside (dN) or a derivative thereof, and optionally at least one additional transduction enhancer (e.g. cyclosporin H (CsH) or a derivative thereof), and/or cell media and/or a population of cells (e.g. HSPCs and/or PBMCs). In one aspect, the present invention provides a kit comprising at least one pyrimidine precursor, and optionally at least one additional transduction enhancer (e.g. cyclosporin H (CsH) or a derivative thereof), and/or cell media and/or a population of cells (e.g. HSPCs and/or PBMCs).

In one aspect, the present invention provides a kit comprising at least one pyrimidine deoxyribonucleoside (dN) or a derivative thereof, and optionally at least one additional transduction enhancer (e.g. cyclosporin H (CsH) or a derivative thereof), and/or cell media and/or a population of cells (e.g. HSPCs and/or PBMCs).

In one aspect, the present invention provides a kit comprising dC or a derivative thereof, and optionally at least one transduction enhancer (e.g. CsH or a derivative thereof), and/or cell media and/or a population of cells (e.g. HSPCs and/or PBMCs).

In one aspect, the present invention provides a kit comprising dT or a derivative thereof, and optionally at least one transduction enhancer (e.g. CsH or a derivative thereof) and/or cell media and/or a population of cells (e.g. HSPCs and/or PBMCs).

In one aspect, the present invention provides a kit comprising orotic acid (OA), and optionally at least one transduction enhancer (e.g. CsH or a derivative thereof), and/or cell media and/or a population of cells (e.g. HSPCs and/or PBMCs).

In one aspect, the present invention provides a kit comprising uridine 5′-monophosphate (UMP), and optionally at least one transduction enhancer (e.g. CsH or a derivative thereof), and/or cell media and/or a population of cells (e.g. HSPCs and/or PBMCs).

### Uses and Methods

### Uses

In one aspect, the present invention provides for use of a deoxyribonucleoside (dN) or a derivative thereof for improving the transduction of cells by viral vectors and/or improving gene editing of cells. Optionally, the dN is used in combination with at least one additional transduction enhancer (e.g. CsH or a derivative thereof).

In one aspect, the present invention provides for use of a dN or a derivative thereof for improving the transduction of cells by viral vectors and/or improving gene editing of cells, wherein the dN or a derivative thereof is contacted with the cells simultaneously, sequentially or separately in combination with at least one additional transduction enhancer (e.g. CsH or a derivative thereof).

In one aspect, the present invention provides for use of a transduction enhancer (e.g. CsH or a derivative thereof) for improving the transduction of cells by viral vectors and/or improving gene editing of cells, wherein the transduction enhancer (e.g CsH or a derivative thereof) is contacted with the cells simultaneously, sequentially or separately in combination with at least one dN or a derivative thereof.

In one aspect, the present invention provides for use of a deoxyribonucleoside (dN) or a derivative thereof for increasing the efficiency of transduction of an isolated population of cells by a viral vector and/or increasing the efficiency of gene editing of an isolated population of cells when transduced by a viral vector. Optionally, the dN is used in combination with at least one additional transduction enhancer (e.g. CsH or a derivative thereof).

In one aspect, the present invention provides for use of a dN or a derivative thereof for increasing the efficiency of transduction of an isolated population of cells by a viral vector and/or increasing the efficiency of gene editing of an isolated population of cells when transduced by a viral vector, wherein the dN or a derivative thereof is contacted with the cells simultaneously, sequentially or separately in combination with at least one additional transduction enhancer (e.g. CsH or a derivative thereof).

In one aspect, the present invention provides for use of a transduction enhancer (e.g. CsH or a derivative thereof) for increasing the efficiency of transduction of an isolated population of cells by a viral vector and/or increasing the efficiency of gene editing of an isolated population of cells when transduced by a viral vector, wherein the transduction enhancer (e.g CsH or a derivative thereof) is contacted with the cells simultaneously, sequentially or separately in combination with at least one dN or a derivative thereof.

In one aspect, the present invention provides for use of a pyrimidine dN or a derivative thereof for improving the transduction of cells by viral vectors and/or improving gene editing of cells. Optionally, the dN is used in combination with at least one additional transduction enhancer (e.g. CsH or a derivative thereof).

In one aspect, the present invention provides for use of a pyrimidine dN or a derivative thereof for improving the transduction of cells by viral vectors and/or improving gene editing of cells, wherein the pyrimidine dN or a derivative thereof is contacted with the cells simultaneously, sequentially or separately in combination with at least one additional transduction enhancer (e.g. CsH or a derivative thereof).

In one aspect, the present invention provides for use of a transduction enhancer (e.g. CsH or a derivative thereof) for improving the transduction of cells by viral vectors and/or improving gene editing of cells, wherein the transduction enhancer (e.g. CsH or a derivative thereof) is contacted with the cells simultaneously, sequentially or separately in combination with at least one pyrimidine dN or a derivative thereof.

In one aspect, the present invention provides for use of a pyrimidine dN or a derivative thereof for increasing the efficiency of transduction of an isolated population of cells by a viral vector and/or increasing the efficiency of gene editing of an isolated population of cells when transduced by a viral vector. Optionally, the dN is used in combination with at least one additional transduction enhancer (e.g. CsH or a derivative thereof).

In one aspect, the present invention provides for use of a pyrimidine dN or a derivative thereof for increasing the efficiency of transduction of an isolated population of cells by a viral vector and/or increasing the efficiency of gene editing of an isolated population of cells when transduced by a viral vector, wherein the pyrimidine dN or a derivative thereof is contacted with the cells simultaneously, sequentially or separately in combination with at least one additional transduction enhancer (e.g. CsH or a derivative thereof).

In one aspect, the present invention provides for use of a transduction enhancer (e.g. CsH or a derivative thereof) for increasing the efficiency of transduction of an isolated population of cells by a viral vector and/or increasing the efficiency of gene editing of an isolated population of cells when transduced by a viral vector, wherein the transduction enhancer (e.g. CsH or a derivative thereof) is contacted with the cells simultaneously, sequentially or separately in combination with at least one pyrimidine dN or a derivative thereof.

In one aspect, the present invention provides for use of dC or a derivative thereof for increasing the efficiency of transduction of an isolated population of cells by a viral vector and/or increasing the efficiency of gene editing of an isolated population of cells when transduced by a viral vector. Optionally, wherein dC or a derivative thereof is contacted with the cells simultaneously, sequentially or separately in combination with at least one additional transduction enhancer (e.g. CsH or a derivative thereof).

In one aspect, the present invention provides for use of a transduction enhancer (e.g. CsH or a derivative thereof) for increasing the efficiency of transduction of an isolated population of cells by a viral vector and/or increasing the efficiency of gene editing of an isolated population of cells when transduced by a viral vector, wherein the transduction enhancer (e.g. CsH or a derivative thereof) is contacted with the cells simultaneously, sequentially or separately in combination with dC or a derivative thereof.

In one aspect, the present invention provides for use of dT or a derivative thereof for increasing the efficiency of transduction of an isolated population of cells by a viral vector and/or increasing the efficiency of gene editing of an isolated population of cells when transduced by a viral vector. Optionally, wherein dT or a derivative thereof is contacted with the cells simultaneously, sequentially or separately in combination with at least one additional transduction enhancer (e.g. CsH or a derivative thereof).

In one aspect, the present invention provides for use of a transduction enhancer (e.g. CsH or a derivative thereof) for increasing the efficiency of transduction of an isolated population of cells by a viral vector and/or increasing the efficiency of gene editing of an isolated population of cells when transduced by a viral vector, wherein the transduction enhancer (e.g. CsH or a derivative thereof) is contacted with the cells simultaneously, sequentially or separately in combination with dT or a derivative thereof.

In further aspects, the present invention provides the above uses in which the deoxyribonucleoside (dN) or a derivative thereof is replaced with a pyrimidine precursor, for example as described herein (e.g. orotic acid (OA) or uridine 5′-monophosphate (UMP)).

### Methods of Transducing a Population of Cells

In one aspect, the present invention provides a method of transducing a population of cells comprising the step of contacting the population of cells with at least one deoxyribonucleoside (dN) or a derivative thereof. Suitably, the method further comprises transducing the population of cells with a viral vector. Optionally, the population of cells is contacted with the at least one dN in combination with at least one additional transduction enhancer (e.g. CsH or a derivative thereof).

In one aspect, the present invention provides a method of transducing a population of cells comprising the steps of:
(a) contacting the population of cells with at least one pyrimidine dN or a derivative thereof, optionally in combination with at least one additional transduction enhancer (e.g. CsH or a derivative thereof); and
(b) transducing the population of cells with a viral vector.

The method of transduction may increase the efficiency of transduction of the population of cells by the viral vector and/or increase the efficiency of gene editing of the population of cells when transduced by the viral vector

In one embodiment, the pyrimidine dN or a derivative thereof is deoxycytidine (dC) or a derivative thereof and/or thymidine (dT) or a derivative thereof. In one embodiment, the pyrimidine dN or a derivative thereof is dC or a derivative thereof.

In one aspect, the present invention provides a method of transducing a population of cells comprising the steps of:
(a) contacting the population of cells with dC or a derivative thereof, optionally in combination with at least one additional transduction enhancer (e.g. CsH or a derivative thereof); and
(b) transducing the population of cells with a viral vector.

In one aspect, the present invention provides a method of transducing a population of cells comprising the steps of:
(a) contacting the population of cells with dT or a derivative thereof, optionally in combination with at least one additional transduction enhancer (e.g. CsH or a derivative thereof); and
(b) transducing the population of cells with a viral vector.

In one embodiment, the population of cells is contacted with the pyrimidine dN or a derivative thereof in combination with at least one purine dN or a derivative thereof. In one embodiment, the purine dN or a derivative thereof is deoxyadenosine (dA) or a derivative thereof and/or deoxyguanosine (dG) or a derivative thereof.

In one embodiment, the population of cells is contacted with one dN or a derivative thereof. In one embodiment, the population of cells is contacted with a dN mix. Suitably, the population of cells is contacted with a mix of two or more (e.g. 2, 3, 4) dNs or derivatives thereof, three or more (e.g. 3, 4) dNs or derivatives thereof, or four or more dNs or derivatives thereof. In one embodiment, the population of cells is contacted with two dNs or derivatives thereof. In one embodiment, population of cells is contacted with three dNs or derivatives thereof. In one embodiment, population of cells is contacted with four dNs or derivatives thereof. In one embodiment, the population of cells is contacted with dC or a derivative thereof, dT or a derivative thereof, dA or a derivative thereof, and dG or a derivative thereof.

When the population of cells is contacted with more than one agent (e.g. contacted with at least one dN in combination with a least one additional transduction enhancer), the cells may be contacted with the agents simultaneously, sequentially or separately. The term “simultaneous” as used herein means that the agents are used concurrently, i.e. at the same time. The term “sequential” as used herein means that the agents are used one after the other. The term “separate” as used herein means that the agents are used independently of each other but within a time interval that allows the agents to show a combined, preferably synergistic, effect. Thus, using “separately” may permit one agent to be used, for example, within 1 minute, 5 minutes, 10 minutes, 30 minutes or one hour after the other.

In one embodiment, the population of cells is contacted with at least one deoxyribonucleoside (dN) or a derivative thereof simultaneously with at least one additional transduction enhancer (e.g. CsH or a derivative thereof).

The population of cells may be contacted with the agents (e.g. dN or a derivate thereof, a transduction enhancer) before, during, before and during, or after contact with the viral vector, or combinations thereof. In one embodiment, the population of cells is contacted with at least one deoxyribonucleoside before and/or during contact with the viral vector. In one embodiment, the population of cells is contacted with at least one dN (and optionally at least one additional transduction enhancer, e.g. CsH or a derivative thereof) before and during contact with the viral vector.

In further aspects, the present invention provides the above methods in which the deoxyribonucleoside (dN) or a derivative thereof is replaced with a pyrimidine precursor, for example as described herein (e.g. orotic acid (OA) or uridine 5′-monophosphate (UMP)).

The population of cells may be contacted with the agents (e.g. dN or a derivate thereof, a transduction enhancer) for any suitable period of time. In one embodiment, the population of cells is contacted with at least one deoxyribonucleoside (and optionally at least one additional transduction enhancer, e.g. CsH or a derivative thereof) for at least about 30 minutes, at least about 1 hour, or at least about 2 hours before transduction and/or at least about 8 hours, at least about 12 hours, or at least about 16 hours during transduction. In one embodiment, the population of cells is contacted with at least one deoxyribonucleoside (and optionally at least one additional transduction enhancer, e.g. CsH or a derivative thereof) for about 1 hour to about 6 hours, or about 2 hour to about 4 hours before transduction and/or about 12 hours to about 24, or about 16 hours to about 20 hours during transduction.

In preferred embodiments, the population of cells (e.g. comprising or consisting substantially of HSPCs) is not stimulated before and/or during the method of the invention. In one embodiment, the population of cells (e.g. comprising or consisting substantially of HSPCs) is not stimulated before transduction. In one embodiment, the population of cells (e.g. comprising or consisting substantially of HSPCs) is not stimulated during transduction. Quiescent cells (e.g. quiescent HSPCs) typically require extensive cytokine-mediated stimulation for efficient transduction (Zielske, S. P. and Gerson, S. L., 2003. Molecular Therapy, 7 (3), pp. 325-333). Cytokines for stimulating quiescent cells (e.g. quiescent HSPCs) are known to those of skill in the art and include, for example early-acting cytokines such as IL-3, IL-6, stem cell factor (SCF), and Flt-3L. In one embodiment, the population of cells is not contacted with cytokines (e.g. early-acting cytokines) before and/or during transduction. In one embodiment, the population of cells is not contacted with recombinant human stem cell factor (rhSCF), recombinant human thrombopoietin (rhTPO), recombinant human Flt3 ligand (rhFlt3), or recombinant human IL6 (rhIL6) before and/or during transduction.

### Other Methods of Enhancing Transduction

The use or method of the present invention may be combined with any other suitable methods to further increase the efficiency of transduction and/or gene editing.

Exemplary methods for further Increasing the efficiency of transduction and/or gene editing include: contacting the population of cells with one or more additional transduction enhancer; high density culture; viral capsid mutants; alternative Env glycoproteins or VSV-g fusions; pre-stimulation in the presence of cytokines or HSC expansion; combining methods to enhance virus-cell interaction, and spinoculation.

Exemplary methods for further increasing the efficiency of transduction and/or gene editing include use of measles virus glycoprotein pseudotyped viral vector (e.g. viral vector pseudotyped with measles virus glycoproteins hemagglutinin (H) and fusion protein (F)).

In one embodiment, the use or method further comprises one or more of: contacting the population of cells with one or more additional transduction enhancer; high density culture; viral capsid mutants; alternative Env glycoproteins or VSV-g fusions; pre-stimulation in the presence of cytokines or HSC expansion; combining methods to enhance virus-cell interaction; and spinoculation.

In one embodiment, the use or method further comprises use of measles virus glycoprotein pseudotyped viral vector (e.g. viral vector pseudotyped with measles virus glycoproteins hemagglutinin (H) and fusion protein (F)).

In one embodiment, as described above, the population of cells may be further contacted with one or more additional transduction enhancers. The cells may be contacted with one or more additional transduction enhancers at any point prior to or during transduction, for example at the same time as the dN or derivative thereof (and optionally the CsH or a derivative thereof). The cells may be, for example, contacted with one or more additional transduction enhancers at the same time as the pyrimidine precursor (and optionally the CsH or a derivative thereof). In one embodiment, the method comprises contacting the population of cells with one or more additional transduction enhancers before and/or during transduction. In one embodiment, the method comprises contacting the population of cells with one or more additional transduction enhancers simultaneously, sequentially or separately with the dN or derivative thereof. In one embodiment, the method comprises contacting the population of cells with one or more additional transduction enhancers simultaneously, sequentially or separately with the pyrimidine precursor.

In one embodiment, transduction takes place in a high-density culture. High density culture conditions may include a cell density of about 1e6 cells/mL or greater (e.g. 1e6 to 4e6 cells/mL). For example, Uchida N, et al. (2019) Mol Ther Methods Clin Dev 13: 187-196 describes high-efficiency lentiviral transduction of human CD34(+) cells in high-density culture (4e6/mL). In one embodiment, the population of cells is transduced at a cell density of about 1e6 cells/mL or greater or at a cell density of about 1e6 to about 4e6 cells/mL.

In one embodiment, the viral vector comprises viral capsid mutants which increase the efficiency of transduction and/or gene editing. Such viral capsid mutants will be known to those of skill in the art. For example, suitable lentiviral CA mutants are described in Petrillo C, et al (2015) Mol Ther 23: 352-362, including the A88T CA mutant.

In one embodiment, the viral vector comprises alternative Env glycoproteins and/or VSV-g fusions which increase the efficiency of transduction and/or gene editing. Such Env glycoproteins or VSV-g fusions will be known to those of skill in the art. For example, Hanawa H, et al (2002) Mol Ther 5: 242-251 describes a comparison of various envelope proteins for their ability to pseudotype lentiviral vectors and transduce primitive hematopoietic cells from human blood.

In one embodiment, the viral vector comprises measles virus glycoprotein(s) which increase the efficiency of transduction and/or gene editing. Such measles glycoproteins will be known to those of skill in the art (see, for example, Frecha, C. et al. (2009) Blood 114: 3173-3180).

In one embodiment, the viral vector is a measles virus glycoprotein pseudotyped viral vector. In one embodiment, the viral vector is pseudotyped with measles virus glycoproteins hemagglutinin (H) and fusion protein (F).

In one embodiment, the population of cells are pre-stimulated in the presence of cytokines and/or the use or method comprises HSC expansion. Suitable conditions will be known to those of skill in the art. For example, Uchida N, et al (2011) Gene Ther 18: 1078-1086 describes optimal conditions for lentiviral transduction of engrafting human CD34+ cells.

In one embodiment, the use or method comprises a combining method to enhance virus-cell interaction. Suitable conditions will be well known to those of skill in the art. For example, suitable conditions are described in Liu H, et al (2000) Leukemia 14: 307-311.

In one embodiment, the use or method comprises spinoculation. Spinoculation may enhance contact between viral particles and target cells. Suitable conditions will be well known to those of skill in the art. For example, suitable conditions are described in Millington M, et al (2009) PLOS One 4: e6461.

### Population of Cells

In one aspect, the present invention provides a population of cells prepared according to the method of the invention.

In another aspect, the present invention provides a kit comprising the population of cells of the invention.

The population of cells may be an isolated population of cells. In one embodiment, the population of cells comprises, substantially consists of, or consists of: haematopoietic stem and/or progenitor cells (HSPCs), and/or peripheral blood mononuclear cells (PBMCs).

In preferred embodiments the population of cells are quiescent. Quiescence is a reversible state of a cell in which it does not divide but retains the ability to re-enter cell proliferation. Some adult stem cells are maintained in a quiescent state and can be rapidly activated when stimulated.

In one embodiment, the cell subpopulation composition is not substantially affected by the method of the invention. For example, the proportion of cell sub-types may be substantially unaffected by contacting the population of cells with the dN or derivative thereof (alone or in combination with at least one additional transduction enhancer). In one embodiment, the transduced population of cells has substantially the same relative amounts of cell sub-types as the starting population of cells.

### Haematopoietic Stem and Progenitor Cells

In one embodiment, the population of cells comprises, substantially consists of, or consists of haematopoietic stem and/or progenitor cells (HSPCs), e.g. quiescent HSPCs.

A stem cell is able to differentiate into many cell types. A cell that is able to differentiate into all cell types is known as totipotent. In mammals, only the zygote and early embryonic cells are totipotent. Stem cells are found in most, if not all, multicellular organisms. They are characterised by the ability to renew themselves through mitotic cell division and differentiate into a diverse range of specialised cell types. The two broad types of mammalian stem cells are embryonic stem cells that are isolated from the inner cell mass of blastocysts, and adult stem cells that are found in adult tissues. In a developing embryo, stem cells can differentiate into all of the specialised embryonic tissues. In adult organisms, stem cells and progenitor cells act as a repair system for the body, replenishing specialised cells, but also maintaining the normal turnover of regenerative organs, such as blood, skin or intestinal tissues.

Haematopoietic stem cells (HSCs) are multipotent stem cells that may be found, for example, in peripheral blood, bone marrow and umbilical cord blood. HSCs are capable of self-renewal and differentiation into any blood cell lineage. They are capable of recolonising the entire immune system, and the erythroid and myeloid lineages in all the haematopoietic tissues (such as bone marrow, spleen and thymus). They provide for life-long production of all lineages of haematopoietic cells.

Haematopoietic progenitor cells (HPCs) have the capacity to differentiate into a specific type of cell. In contrast to stem cells however, they are already far more specific, they are pushed to differentiate into their “target” cell. A difference between stem cells and progenitor cells is that stem cells can replicate indefinitely, whereas progenitor cells can only divide a limited number of times. Haematopoietic progenitor cells can be rigorously distinguished from HSCs only by functional in vivo assay (i.e. transplantation and demonstration of whether they can give rise to all blood lineages over prolonged time periods).

The haematopoietic stem and/or progenitor cells of the invention may comprise the CD34 cell surface marker (denoted as CD34+). In one embodiment, the haematopoietic stem and/or progenitor cells comprise, substantially consist of, or consist of CD34⁺ cells or CD34⁻ cells. In one embodiment, the haematopoietic stem and/or progenitor cells comprise, substantially consist of, or consist of primitive subtypes. In one embodiment, the haematopoietic stem and/or progenitor cells comprise, substantially consist of, or consist of CD34⁺ cells. In one embodiment, the haematopoietic stem and/or progenitor cells comprise, substantially consist of, or consist of CD34⁺CD133⁻CD90⁻, CD34⁺CD133⁺CD90⁻, and/or CD34⁺CD133⁺CD90⁺ cells. In one embodiment, the haematopoietic stem and/or progenitor cells comprise, substantially consist of, or consist of CD34⁺CD133⁺CD90⁺ cells.

### Haematopoietic Stem and Progenitor Cell (HSPC) Source

A population of haematopoietic stem and/or progenitor cells (HSPCs) may be obtained from a tissue sample.

For example, a population of haematopoietic stem and/or progenitor cells may be obtained from peripheral blood (e.g. adult and foetal peripheral blood), umbilical cord blood, bone marrow, liver or spleen. Preferably, these cells are obtained from peripheral blood or bone marrow. They may be obtained after mobilisation of the cells in vivo by means of growth factor treatment.

Mobilisation may be carried out using, for example, G-CSF, plerixaphor or combinations thereof. Other agents, such as NSAIDs and dipeptidyl peptidase inhibitors, may also be useful as mobilising agents.

With the availability of the stem cell growth factors GM-CSF and G-CSF, most haematopoietic stem cell transplantation procedures are now performed using stem cells collected from the peripheral blood, rather than from the bone marrow. Collecting peripheral blood stem cells provides a bigger graft, does not require that the donor be subjected to general anaesthesia to collect the graft, results in a shorter time to engraftment and may provide for a lower long-term relapse rate.

Bone marrow may be collected by standard aspiration methods (either steady-state or after mobilisation), or by using next-generation harvesting tools (e.g. Marrow Miner).

In addition, haematopoietic stem and progenitor cells may also be derived from induced pluripotent stem cells.

### HSC Characteristics

HSCs are typically of low forward scatter and side scatter profile by flow cytometric procedures. Some are metabolically quiescent, as demonstrated by Rhodamine labelling which allows determination of mitochondrial activity. HSCs may comprise certain cell surface markers such as CD34, CD45, CD133, CD90 and CD49f. They may also be defined as cells lacking the expression of the CD38 and CD45RA cell surface markers. However, expression of some of these markers is dependent upon the developmental stage and tissue-specific context of the HSC. Some HSCs called “side population cells” exclude the Hoechst 33342 dye as detected by flow cytometry. Thus, HSCs have descriptive characteristics that allow for their identification and isolation.

### Negative Markers

CD38 is the most established and useful single negative marker for human HSCs.

Human HSCs may also be negative for lineage markers such as CD2, CD3, CD14, CD16, CD19, CD20, CD24, CD36, CD56, CD66b, CD271 and CD45RA. However, these markers may need to be used in combination for HSC enrichment.

By “negative marker” it is to be understood that human HSCs lack the expression of these markers.

### Positive Markers

CD34 and CD133 are the most useful positive markers for HSCs.

Some HSCs are also positive for lineage markers such as CD90, CD49f and CD93. However, these markers may need to be used in combination for HSC enrichment.

By “positive marker” it is to be understood that human HSCs express these markers.

In one embodiment, the haematopoietic stem and progenitor cells are CD34+CD38− cells.

In one embodiment, the HSPCs are unstimulated (e.g. quiescent) HSPCs. Preferably, the HSPCs are quiescent HSPCs. Unstimulated HSPCs may be HSPCs which are not stimulated prior to and/or during transduction. In one embodiment, the HSPCs are not pre-stimulated HSPCs. Pre-stimulated HSPCs may be HSPCs which are stimulated prior to transduction. In preferred embodiments, the HSPCs are not stimulated before and/or during transduction. In one embodiment, the HSPCs are not stimulated before transduction. In one embodiment, the HSPCs are not stimulated during transduction. Cytokines for stimulating quiescent HSPCs are known to those of skill in the art and include, for example early-acting cytokines such as IL-3, IL-6, stem cell factor (SCF), and Flt-3L. In one embodiment, the (quiescent) HSPCs are not contacted with cytokines (e.g. early-acting cytokines) before and/or during transduction. In one embodiment, the (quiescent) HSPCs are not contacted with recombinant human stem cell factor (rhSCF), recombinant human thrombopoietin (rhTPO), recombinant human Flt3 ligand (rhFlt3), or recombinant human IL6 (rhIL6) before and/or during transduction.

### Peripheral Blood Mononuclear Cells and T Cells

In one embodiment, the population of cells comprises, substantially consists of, or consists of peripheral blood mononuclear cells (PBMCs). Peripheral blood mononuclear cells (PBMCs) are blood cells with round nuclei, such as monocytes, lymphocytes, and macrophages.

The PBMCs of the invention may, for example, not display the CD14 cell surface marker (denoted as CD14-). Cluster of differentiation 14 (CD14) has been described as a monocyte/macrophage differentiation antigen on the surface of myeloid lineage and has been commonly used in normal tissue or blood as a marker for myeloid cells.

In one embodiment, the population of cells comprises, substantially consists of, or consists of T cells. T cells (or T lymphocytes) are a type of lymphocyte that play a central role in cell-mediated immunity. They can be distinguished from other lymphocytes, such as B cells and natural killer cells (NK cells), by the presence of a T-cell receptor (TCR) on the cell surface.

In one embodiment, the T cells are resting T cells. Resting CD4+ T cells are quiescent. In one embodiment, the T cells are unstimulated T cells. Once stimulated, these resting T cells proliferate and generate a large clone of antigen-specific cells. In one embodiment, the T cells are CD4+ T cells. In one embodiment, the T cells are CD3+ T cells. In one embodiment, the T cells are CD8+ T cells.

In one embodiment, the T cells are resting CD3⁺ T cells. In one embodiment, the T cells are Stem memory T cells; Central Memory T cells; Effector Memory T cells; and/or terminally differentiated effector memory T cells.

### Isolation and Enrichment of Populations of Cells

The term “isolated population” of cells as used herein may refer to the population of cells having been previously removed from the body. An isolated population of cells may be cultured and manipulated ex vivo or in vitro using standard techniques known in the art. An isolated population of cells may later be reintroduced into a subject. Said subject may be the same subject from which the cells were originally isolated or a different subject.

A population of cells may be purified selectively for cells that exhibit a specific phenotype or characteristic, and from other cells which do not exhibit that phenotype or characteristic, or exhibit it to a lesser degree. For example, a population of cells that expresses a specific marker (such as CD34) may be purified from a starting population of cells. Alternatively, or in addition, a population of cells that does not express another marker (such as CD38) may be purified.

By “enriching” a population of cells for a certain type of cells it is to be understood that the concentration of that type of cells is increased within the population. The concentration of other types of cells may be concomitantly reduced.

Purification or enrichment may result in the population of cells being substantially pure of other types of cell.

Purifying or enriching for a population of cells expressing a specific marker (e.g. CD34 or CD38) may be achieved by using an agent that binds to that marker, preferably substantially specifically to that marker.

An agent that binds to a cellular marker may be an antibody, for example an anti-CD34 or anti-CD38 antibody.

The term “antibody” refers to complete antibodies or antibody fragments capable of binding to a selected target, and including Fv, ScFv, F(ab′) and F(ab′)₂, monoclonal and polyclonal antibodies, engineered antibodies including chimeric, CDR-grafted and humanised antibodies, and artificially selected antibodies produced using phage display or alternative techniques.

In addition, alternatives to classical antibodies may also be used in the invention, for example “avibodies”, “avimers”, “anticalins”, “nanobodies” and “DARPins”.

The agents that bind to specific markers may be labelled so as to be identifiable using any of a number of techniques known in the art. The agent may be inherently labelled, or may be modified by conjugating a label thereto. By “conjugating” it is to be understood that the agent and label are operably linked. This means that the agent and label are linked together in a manner which enables both to carry out their function (e.g. binding to a marker, allowing fluorescent identification or allowing separation when placed in a magnetic field) substantially unhindered. Suitable methods of conjugation are well known in the art and would be readily identifiable by the skilled person.

A label may allow, for example, the labelled agent and any cell to which it is bound to be purified from its environment (e.g. the agent may be labelled with a magnetic bead or an affinity tag, such as avidin), detected or both. Detectable markers suitable for use as a label include fluorophores (e.g. green, cherry, cyan and orange fluorescent proteins) and peptide tags (e.g. His tags, Myc tags, FLAG tags and HA tags).

A number of techniques for separating a population of cells expressing a specific marker are known in the art. These include magnetic bead-based separation technologies (e.g. closed-circuit magnetic bead-based separation), flow cytometry, fluorescence-activated cell sorting (FACS), affinity tag purification (e.g. using affinity columns or beads, such biotin columns to separate avidin-labelled agents) and microscopy-based techniques.

It may also be possible to perform the separation using a combination of different techniques, such as a magnetic bead-based separation step followed by sorting of the resulting population of cells for one or more additional (positive or negative) markers by flow cytometry.

Clinical grade separation may be performed, for example, using the CliniMACS® system (Miltenyi). This is an example of a closed-circuit magnetic bead-based separation technology.

It is also envisaged that dye exclusion properties (e.g. side population or rhodamine labelling) or enzymatic activity (e.g. ALDH activity) may be used to enrich for haematopoietic stem cells.

### Gene Editing

The term “gene editing” refers to a type of genetic engineering in which a nucleic acid is inserted, deleted or replaced in a cell. Gene editing may be achieved using engineered nucleases, which may be targeted to a desired site in a polynucleotide (e.g. a genome). Such nucleases may create site-specific double-strand breaks at desired locations, which may then be repaired through non-homologous end-joining (NHEJ) or homologous recombination (HR), resulting in targeted mutations.

Such nucleases may be delivered to a target cell using viral vectors. The present invention provides methods of increasing the efficiency of the gene editing process.

Examples of suitable nucleases known in the art include zinc finger nucleases (ZFNs), transcription activator like effector nucleases (TALENs), and the clustered regularly interspaced short palindromic repeats (CRISPR)/Cas system (Gaj, T. et al. (2013) Trends Biotechnol. 31: 397-405; Sander, J. D. et al. (2014) Nat. Biotechnol. 32: 347-55).

Meganucleases (Silve, G. et al. (2011) Cur. Gene Ther. 11: 11-27) may also be employed as suitable nucleases for gene editing.

The CRISPR/Cas system is an RNA-guided DNA binding system (van der Oost et al. (2014) Nat. Rev. Microbiol. 12: 479-92), wherein the guide RNA (gRNA) may be selected to enable a Cas9 domain to be targeted to a specific sequence. Methods for the design of gRNAs are known in the art. Furthermore, fully orthogonal Cas9 proteins, as well as Cas9/gRNA ribonucleoprotein complexes and modifications of the gRNA structure/composition to bind different proteins, have been developed to simultaneously and directionally target different effector domains to desired genomic sites of the cells (Esvelt et al. (2013) Nat. Methods 10: 1116-21; Zetsche, B. et al. (2015) Cell pii: S0092-8674(15)01200-3; Dahlman, J. E. et al. (2015) Nat. Biotechnol. 2015 Oct. 5. doi: 10.1038/nbt.3390.; Zalatan, J. G. et al. (2015) Cell 160: 339-50; Paix, A. et al. (2015) Genetics 201: 47-54), and are suitable for use in the invention.

### Viral Vectors

A vector is a tool that allows or facilitates the transfer of an entity from one environment to another. The vectors used to transduce the cells in the present invention are viral vectors.

In one embodiment, the viral vectors are retroviral vectors. In a preferred embodiment, the viral vectors are lentiviral vectors.

In one embodiment, the lentiviral vectors are derived from HIV-1, HIV-2, SIV, FIV, BIV, EIAV, CAEV or visna lentivirus. In one embodiment, the viral vector is a gamma-retroviral vector.

The vector of the present invention may be in the form of a viral vector particle. In one embodiment, the viral vector is pseudotyped to enter cells via an endocytosis-dependent mechanism and/or the viral vector is a VSV-g pseudotyped vector. In one embodiment, the viral vector is pseudotyped to enter cells via an endocytosis-dependent mechanism. In one embodiment, the viral vector is a VSV-g pseudotyped vector. In one embodiment, the viral vector is a measles virus glycoprotein pseudotyped viral vector. In one embodiment, the viral vector is pseudotyped with measles virus glycoproteins hemagglutinin (H) and fusion protein (F). In one embodiment, the viral vector is not an adeno-associated virus (AAV) vector.

By “vector derived from” a certain type of virus, it is to be understood that the vector comprises at least one component part derivable from that type of virus.

### Retroviral and Lentiviral Vectors

A retroviral vector may be derived from or may be derivable from any suitable retrovirus. A large number of different retroviruses have been identified. Examples include murine leukaemia virus (MLV), human T cell leukaemia virus (HTLV), mouse mammary tumour virus (MMTV), Rous sarcoma virus (RSV), Fujinami sarcoma virus (FuSV), Moloney murine leukaemia virus (Mo-MLV), FBR murine osteosarcoma virus (FBR MSV), Moloney murine sarcoma virus (Mo-MSV), Abelson murine leukaemia virus (A-MLV), avian myelocytomatosis virus-29 (MC29) and avian erythroblastosis virus (AEV). A detailed list of retroviruses may be found in Coffin, J. M. et al. (1997) Retroviruses, Cold Spring Harbour Laboratory Press, 758-63.

Retroviruses may be broadly divided into two categories, “simple” and “complex”. Retroviruses may be even further divided into seven groups. Five of these groups represent retroviruses with oncogenic potential. The remaining two groups are the lentiviruses and the spumaviruses. A review of these retroviruses is presented in Coffin, J. M. et al. (1997) Retroviruses, Cold Spring Harbour Laboratory Press, 758-63.

The basic structure of retrovirus and lentivirus genomes share many common features such as a 5′ LTR and a 3′ LTR. Between or within these are located a packaging signal to enable the genome to be packaged, a primer binding site, integration sites to enable integration into a host cell genome, and gag, pol and env genes encoding the packaging components—these are polypeptides required for the assembly of viral particles. Lentiviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration and transcription. LTRs also serve as enhancer-promoter sequences and can control the expression of the viral genes.

The LTRs themselves are identical sequences that can be divided into three elements: U3, R and U5. U3 is derived from the sequence unique to the 3′ end of the RNA. R is derived from a sequence repeated at both ends of the RNA. U5 is derived from the sequence unique to the 5′ end of the RNA. The sizes of the three elements can vary considerably among different retroviruses.

In a defective retroviral vector genome gag, pol and env may be absent or not functional.

In a typical retroviral vector, at least part of one or more protein coding regions essential for replication may be removed from the virus. This makes the viral vector replication-defective. Portions of the viral genome may also be replaced by a library encoding candidate modulating moieties operably linked to a regulatory control region and a reporter moiety in the vector genome in order to generate a vector comprising candidate modulating moieties which is capable of transducing a target host cell and/or integrating its genome into a host genome.

Lentivirus vectors are part of the larger group of retroviral vectors. A detailed list of lentiviruses may be found in Coffin, J. M. et al. (1997) Retroviruses, Cold Spring Harbour Laboratory Press, 758-63. In brief, lentiviruses can be divided into primate and non-primate groups. Examples of primate lentiviruses include but are not limited to human immunodeficiency virus (HIV), the causative agent of human acquired immunodeficiency syndrome (AIDS); and simian immunodeficiency virus (SIV). Examples of non-primate lentiviruses include the prototype “slow virus” visna/maedi virus (VMV), as well as the related caprine arthritis-encephalitis virus (CAEV), equine infectious anaemia virus (EIAV), and the more recently described feline immunodeficiency virus (FIV) and bovine immunodeficiency virus (BIV).

The lentivirus family differs from other retroviruses in that lentiviruses have the capability to infect both dividing and non-dividing cells (Lewis, P et al. (1992) EMBO J. 11: 3053-8; Lewis, P. F. et al. (1994) J. Virol. 68: 510-6). In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Preferably, that component part is involved in the biological mechanisms by which the vector infects cells, expresses genes or is replicated.

The lentiviral vector may be a “primate” vector. The lentiviral vector may be a “non-primate” vector (i.e. derived from a virus which does not primarily infect primates, especially humans). Examples of non-primate lentiviruses may be any member of the family of lentiviridae which does not naturally infect a primate.

As examples of lentivirus-based vectors, HIV-1- and HIV-2-based vectors are described below.

The HIV-1 vector contains cis-acting elements that are also found in simple retroviruses. It has been shown that sequences that extend into the gag open reading frame are important for packaging of HIV-1. Therefore, HIV-1 vectors often contain the relevant portion of gag in which the translational initiation codon has been mutated. In addition, most HIV-1 vectors also contain a portion of the env gene that includes the RRE. Rev binds to RRE, which permits the transport of full-length or singly spliced mRNAs from the nucleus to the cytoplasm. In the absence of Rev and/or RRE, full-length HIV-1 RNAs accumulate in the nucleus. Alternatively, a constitutive transport element from certain simple retroviruses such as Mason-Pfizer monkey virus can be used to relieve the requirement for Rev and RRE. Efficient transcription from the HIV-1 LTR promoter requires the viral protein Tat.

Most HIV-2-based vectors are structurally very similar to HIV-1 vectors. Similar to HIV-1-based vectors, HIV-2 vectors also require RRE for efficient transport of the full-length or singly spliced viral RNAs.

In one system, the vector and helper constructs are from two different viruses, and the reduced nucleotide homology may decrease the probability of recombination. In addition to vectors based on the primate lentiviruses, vectors based on FIV have also been developed as an alternative to vectors derived from the pathogenic HIV-1 genome. The structures of these vectors are also similar to the HIV-1 based vectors.

Preferably, the viral vector used in the present invention has a minimal viral genome.

By “minimal viral genome” it is to be understood that the viral vector has been manipulated so as to remove the non-essential elements and to retain the essential elements in order to provide the required functionality to infect, transduce and deliver a nucleotide sequence of interest to a target host cell. Further details of this strategy can be found in WO 1998/017815.

Preferably the plasmid vector used to produce the viral genome within a host cell/packaging cell will have sufficient lentiviral genetic information to allow packaging of an RNA genome, in the presence of packaging components, into a viral particle which is capable of infecting a target cell, but is incapable of independent replication to produce infectious viral particles within the final target cell. Preferably, the vector lacks a functional gag-pol and/or env gene and/or other genes essential for replication.

The plasmid vector used to produce the viral genome within a host cell/packaging cell may also include transcriptional regulatory control sequences operably linked to the lentiviral genome to direct transcription of the genome in a host cell/packaging cell. These regulatory sequences may be the natural sequences associated with the transcribed viral sequence (i.e. the 5′ U3 region), or they may be a heterologous promoter, such as another viral promoter (e.g. the CMV promoter).

The vectors may be self-inactivating (SIN) vectors in which the viral enhancer and promoter sequences have been deleted. SIN vectors can be generated and transduce non-dividing cells in vivo with an efficacy similar to that of wild-type vectors. The transcriptional inactivation of the long terminal repeat (LTR) in the SIN provirus should prevent mobilisation by replication-competent virus. This should also enable the regulated expression of genes from internal promoters by eliminating any cis-acting effects of the LTR.

The vectors may be integration-defective. Integration defective lentiviral vectors (IDLVs) can be produced, for example, either by packaging the vector with catalytically inactive integrase (such as an HIV integrase bearing the D64V mutation in the catalytic site; Naldini, L. et al. (1996) Science 272: 263-7; Naldini, L. et al. (1996) Proc. Natl. Acad. Sci. USA 93: 11382-8; Leavitt, A. D. et al. (1996) J. Virol. 70: 721-8) or by modifying or deleting essential att sequences from the vector LTR (Nightingale, S. J. et al. (2006) Mol. Ther. 13: 1121-32), or by a combination of the above. In one embodiment, the vector is integrase-defective.

### HIV-derived Vectors

In one embodiment, the viral vector is an HIV-derived vector. HIV-derived vectors for use in the present invention are not particularly limited in terms of HIV strain. Numerous examples of sequences of HIV strains may be found at the HIV Sequence Database (http://www.hiv.lanl.gov/content/index).

For example, a HIV-1-derived vector may be derived from any of the HIV-1 strains NL4-3, IIIB_LAI or HXB2_LAI (X4-tropic), or BAL (R5-tropic), or a chimaera thereof. Preferably, HIV-1-derived vectors are derived from the pMDLg/pRRE Gag-Pol-expressing packaging construct (U.S. Pat. Nos. 7,629,153; 8,652,837; Naldini, L. et al. (1996) Science 272: 263-7; Follenzi, A. et al. (2002) Methods Enzymol. 346: 454-65).

A HIV-2-derived vector may be derived, for example, from the HIV-2 strain ROD.

### AAV Vectors

In one embodiment, the viral vector is an adeno-associated viral (AAV) vector. In one embodiment, the viral vector is an AAV vector particle.

The AAV vector or AAV vector particle may comprise an AAV genome or a fragment or derivative thereof. An AAV genome is a polynucleotide sequence, which may encode functions needed for production of an AAV particle. These functions include those operating in the replication and packaging cycle of AAV in a host cell, including encapsidation of the AAV genome into an AAV particle. Naturally occurring AAVs are replication-deficient and rely on the provision of helper functions in trans for completion of a replication and packaging cycle. Accordingly, the AAV genome is typically replication-deficient.

The AAV genome may be in single-stranded form, either positive or negative-sense, or alternatively in double-stranded form. The use of a double-stranded form allows bypass of the DNA replication step in the target cell and so can accelerate transgene expression.

AAVs occurring in nature may be classified according to various biological systems. The AAV genome may be from any naturally derived serotype, isolate or clade of AAV. Suitably, the AAV genome is derivatised for the purpose of administration to patients. Such derivatisation is standard in the art and the invention encompasses the use of any known derivative of an AAV genome, and derivatives which could be generated by applying techniques known in the art. The AAV genome may be a derivative of any naturally occurring AAV. Suitably, the AAV genome is a derivative of AAV1, AAV2, AAV3, AAV4, AAV5, AAV6, AAV7, AAV8, AAV9, AAV10, or AAV11.

The AAV vector particle may be encapsidated by capsid proteins. Suitably, the AAV vector particles may be transcapsidated forms wherein an AAV genome or derivative having an ITR of one serotype is packaged in the capsid of a different serotype. The AAV vector particle also includes mosaic forms wherein a mixture of unmodified capsid proteins from two or more different serotypes makes up the viral capsid. The AAV vector particle also includes chemically modified forms bearing ligands adsorbed to the capsid surface. For example, such ligands may include antibodies for targeting a particular cell surface receptor.

### Nucleotide of Interest

The vector used in the present invention preferably comprises a nucleotide of interest (NOI). Preferably, the nucleotide of interest gives rise to a therapeutic effect.

Suitable NOIs include, but are not limited to sequences encoding enzymes, cytokines, chemokines, hormones, antibodies, anti-oxidant molecules, engineered immunoglobulin-like molecules, single chain antibodies, fusion proteins, immune co-stimulatory molecules, immunomodulatory molecules, anti-sense RNA, microRNA, shRNA, siRNA, guide RNA (gRNA, e.g. used in connection with a CRISPR/Cas system), ribozymes, miRNA target sequences, a transdomain negative mutant of a target protein, toxins, conditional toxins, antigens, tumour suppressor proteins, growth factors, transcription factors, membrane proteins, surface receptors, anti-cancer molecules, vasoactive proteins and peptides, anti-viral proteins and ribozymes, and derivatives thereof (such as derivatives with an associated reporter group). The NOIs may also encode pro-drug activating enzymes.

An example of a NOI is the beta-globin chain which may be used for gene therapy of thalassemia/sickle cell disease.

NOIs also include those useful for the treatment of other diseases requiring non-urgent/elective gene correction in the myeloid lineage such as: chronic granulomatous disease (CGD, e.g. the gp91phox transgene), leukocyte adhesion defects, other phagocyte disorders in patients without ongoing severe infections and inherited bone marrow failure syndromes (e.g. Fanconi anaemia), as well as primary immunodeficiencies (SCIDs).

NOIs also include those useful in the treatment of lysosomal storage disorders and immunodeficiencies.

The applicability of the invention to T cells also facilitates its application in cell therapies that are based on infusion of modified T cells into patients, including anti-cancer strategies (such as using engineered CAR-T cells) and approaches based on infusion of universal donor T cells. NOIs may therefore also include, for example, chimeric antigen receptors (CARs).

### Pharmaceutical Composition

The cells of the invention may be formulated for administration to subjects with a pharmaceutically acceptable carrier, diluent or excipient. Suitable carriers and diluents include isotonic saline solutions, for example phosphate-buffered saline, and potentially contain human serum albumin.

Handling of the cell therapy product is preferably performed in compliance with FACT-JACIE International Standards for cellular therapy.

### Haematopoietic Stem and/or Progenitor Cell Transplantation

The present invention provides a population of cells, for example a population of HSPCs or a population of PBMCs (e.g. T cells), prepared according to a method of the invention for use in therapy, for example for use in gene therapy.

The use may be as part of a cell transplantation procedure, for example a haematopoietic stem and/or progenitor cell transplantation procedure.

Haematopoietic stem cell transplantation (HSCT) is the transplantation of blood stem cells derived from the bone marrow (in this case known as bone marrow transplantation) or blood. Stem cell transplantation is a medical procedure in the fields of haematology and oncology, most often performed for people with diseases of the blood or bone marrow, or certain types of cancer.

Many recipients of HSCTs are multiple myeloma or leukaemia patients who would not benefit from prolonged treatment with, or are already resistant to, chemotherapy. Candidates for HSCTs include paediatric cases where the patient has an inborn defect such as severe combined immunodeficiency or congenital neutropenia with defective stem cells, and also children or adults with aplastic anaemia who have lost their stem cells after birth. Other conditions treated with stem cell transplants include sickle-cell disease, myelodysplastic syndrome, neuroblastoma, lymphoma, Ewing's Sarcoma, Desmoplastic small round cell tumour and Hodgkin's disease. More recently non-myeloablative, or so-called “mini transplant”, procedures have been developed that require smaller doses of preparative chemotherapy and radiation. This has allowed HSCT to be conducted in the elderly and other patients who would otherwise be considered too weak to withstand a conventional treatment regimen.

In one embodiment, a population of haematopoietic stem and/or progenitor cells prepared according to a method of the invention is administered as part of an autologous stem cell transplant procedure.

In another embodiment, a population of haematopoietic stem and/or progenitor cells prepared according to a method of the invention is administered as part of an allogeneic stem cell transplant procedure.

The term “autologous stem cell transplant procedure” as used herein refers to a procedure in which the starting population of cells (which are then transduced according to a method of the invention) is obtained from the same subject as that to which the transduced cell population is administered. Autologous transplant procedures are advantageous as they avoid problems associated with immunological incompatibility and are available to subjects irrespective of the availability of a genetically matched donor.

The term “allogeneic stem cell transplant procedure” as used herein refers to a procedure in which the starting population of cells (which are then transduced according to a method of the invention) is obtained from a different subject as that to which the transduced cell population is administered. Preferably, the donor will be genetically matched to the subject to which the cells are administered to minimise the risk of immunological incompatibility.

Suitable doses of transduced cell populations are such as to be therapeutically and/or prophylactically effective. The dose to be administered may depend on the subject and condition to be treated, and may be readily determined by a skilled person.

Haematopoietic progenitor cells provide short term engraftment. Accordingly, gene therapy by administering transduced haematopoietic progenitor cells would provide a non-permanent effect in the subject. For example, the effect may be limited to 1-6 months following administration of the transduced haematopoietic progenitor cells. An advantage of this approach would be better safety and tolerability, due to the self-limited nature of the therapeutic intervention.

Such haematopoietic progenitor cell gene therapy may be suited to treatment of acquired disorders, for example cancer, where time-limited expression of a (potentially toxic) anti-cancer nucleotide of interest may be sufficient to eradicate the disease.

The invention may be useful in the treatment of the disorders listed in WO 1998/005635. For ease of reference, part of that list is now provided: cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the invention may be useful in the treatment of the disorders listed in WO 1998/007859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); anti-inflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the invention may be useful in the treatment of the disorders listed in WO 1998/009985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

In addition, or in the alternative, the invention may be useful in the treatment of B-thalassemia, chronic granulomatous disease, metachromatic leukodystrophy, mucopolysaccharidoses disorders and other lysosomal storage disorders.

As mentioned above, the applicability of the invention to T cells also facilitates its application in cell therapies that are based on infusion of modified T cells into patients, including anti-cancer strategies (such as using engineered CAR-T cells) and approaches based on infusion of universal donor T cells. Thus, in addition, or in the alternative, the invention may be useful in the prevention of graft-versus-host disease.

### Method of Treatment

In one aspect, the present invention provides a deoxyribonucleoside (dN) or a derivative thereof for use in therapy. In one aspect, the present invention provides a pyrimidine precursor for use in therapy. In one embodiment, the therapy is gene therapy, preferably haematopoietic stem and/or progenitor cell gene therapy. In one embodiment, the therapy is a cell therapy, preferably a CAR-T cell therapy. The therapy be used to treat any disorder described herein.

It is to be appreciated that all references herein to treatment include curative, palliative and prophylactic treatment; although in the context of the invention references to preventing are more commonly associated with prophylactic treatment. The treatment of mammals, particularly humans, is preferred. Both human and veterinary treatments are within the scope of the invention.

The skilled person will understand that they can combine all features of the invention disclosed herein without departing from the scope of the invention as disclosed.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### Embodiments

The present invention also provides further aspects as defined in the following numbered paragraphs:
1. A combination of at least one deoxyribonucleoside (dN) or a derivative thereof and cyclosporin H (CsH) or a derivative thereof.
2. The combination according to paragraph 1, wherein the at least one dN or a derivative thereof comprises at least one pyrimidine dN or a derivative thereof.
3. The combination according to paragraph 1 or 2, wherein the at least one dN or a derivative thereof comprises deoxycytidine (dC) or a derivative thereof and/or thymidine (dT) or a derivative thereof, preferably wherein the at least one dN or a derivative thereof comprises dC or a derivative thereof.
4. The combination according to paragraph 2 or 3, wherein the at least one dN or a derivative thereof further comprises at least one purine dN or a derivative thereof.
5. The combination according to any of paragraphs 2 to 4, wherein the at least one dN or a derivative thereof further comprises deoxyadenosine (dA) or a derivative thereof and/or deoxyguanosine (dG) or a derivative thereof.
6. The combination according to any preceding paragraph, wherein the at least one dN or a derivative thereof comprises or consists of dC or a derivative thereof, dT or a derivative thereof, dA or a derivative thereof, and dG or a derivative thereof.
7. The combination according to any preceding paragraph, wherein at least one dN or a derivative thereof and CsH or a derivative thereof are in a dN:CsH molar ratio of from about 2:1 to about 200:1, preferably wherein at least one dN or a derivative thereof and CsH or a derivative thereof are in a dN:CsH molar ratio of from about 10:1 to about 100:1.
8. The combination according to any preceding paragraph, wherein at least one pyrimidine dN or a derivative thereof and CsH or a derivative thereof are in a dN:CsH molar ratio of from about 2:1 to about 200:1, preferably wherein at least one pyrimidine dN or a derivative thereof and CsH or a derivative thereof are in a dN:CsH molar ratio of from about 10:1 to about 100:1.
9. The combination according to any preceding paragraph, wherein dC or a derivative thereof and CsH or a derivative thereof are in a dN:CsH molar ratio of from about 2:1 to about 200:1 and/or dT or a derivative thereof and CsH or a derivative thereof are in a dN:CsH molar ratio of from about 2:1 to about 200:1, preferably wherein dC or a derivative thereof and CsH or a derivative thereof are in a dN:CsH molar ratio of from about 10:1 to about 100:1 and/or dT or a derivative thereof and CsH or a derivative thereof are in a dN:CsH molar ratio of from about 10:1 to about 100:1.
10. The combination according to paragraph 8 or 9, wherein dA or a derivative thereof and/or dG or a derivative thereof are in a dN:CsH molar ratio of from about 2:1 to about 200:1, preferably wherein the dA or a derivative thereof and/or dG or a derivative thereof are in a dN:CsH molar ratio of from about 10:1 to about 100:1.
11. The combination according to any preceding paragraph, wherein at least one dN or a derivative thereof is at a concentration of from about 25 μM to about 1000 μM, preferably from about 100 μM to about 500 μM.
12. The combination according to any preceding paragraph, wherein at least one pyrimidine dN or a derivative thereof is at a concentration of from about 25 μM to about 1000 μM, preferably from about 100 μM to about 500 μM.
13. The combination according to any preceding paragraph, wherein dC or a derivative thereof is at a concentration of from about 25 μM to about 1000 μM, preferably from about 100 μM to about 500 μM, and/or dT or a derivative thereof is at a concentration of from about 25 μM to about 1000 μM, preferably from about 100 μM to about 500 μM.
14. The combination according to paragraph 12 or 13, wherein dA or a derivative thereof is at a concentration of from about 25 μM to about 1000 μM, preferably from about 100 μM to about 500 μM, and/or dG or a derivative thereof is at a concentration of from about 25 μM to about 1000 μM, preferably from about 100 μM to about 500 μM.
15. The combination according to any preceding paragraph, wherein dC or a derivative thereof is at a concentration of from about 25 μM to about 1000 μM, dT or a derivative thereof is at a concentration of from about 25 μM to about 1000 μM, dA or a derivative thereof is at a concentration of from about 25 μM to about 1000 μM, and dG or a derivative thereof is at a concentration of from about 25 μM to about 1000 μM.
16. The combination according to any preceding paragraph, wherein CsH or derivative thereof is at a concentration of from about 1 μM to about 50 μM.
17. A composition comprising a combination according to any of paragraphs 1 to 16, optionally wherein the composition is a cell culture medium or a media supplement, preferably wherein the composition is a cell culture medium.
18. A kit comprising a combination according to any of paragraphs 1 to 16 or a composition according to paragraph 17, and optionally one or more further compositions for cell transduction and/or optionally one or more further agents for cell transduction.
19. Use of a combination according to any of paragraphs 1 to 16, a composition according to paragraph 17, or a kit according to paragraph 18, for increasing the efficiency of transduction of an isolated population of cells by a viral vector and/or increasing the efficiency of gene editing of an isolated population of cells when transduced by a viral vector.
20. A method of transducing a population of cells comprising the steps of:
   (a) contacting the population of cells with a combination according to any of paragraphs 1 to 16 or a composition according to paragraph 17; and
   (b) transducing the population of cells with a viral vector.
21. The method of paragraph 20, wherein the method is an in vitro method or an ex vivo method.
22. Use of a deoxyribonucleoside (dN) or a derivative thereof for increasing the efficiency of transduction of an isolated population of cells by a viral vector and/or increasing the efficiency of gene editing of an isolated population of cells when transduced by a viral vector.
23. A method of transducing a population of cells comprising the steps of:
   (a) contacting the population of cells with at least one deoxyribonucleoside (dN) or a derivative thereof; and
   (b) transducing the population of cells with a viral vector;
wherein the population of cells comprises or consists substantially of: (i) unstimulated haematopoietic stem and/or progenitor cells (HSPCs); and/or (ii) CD14 peripheral blood mononuclear cells (PBMCs).
24. The method of paragraph 23, wherein the method is an in vitro method or an ex vivo method.
25. The use according to paragraph 22, or the method according to paragraph 23 or 24, wherein the dN or a derivative thereof is a pyrimidine dN or a derivative thereof.
26. The use according to paragraph 22 or 25, or the method according to any of paragraphs 23 to 25, wherein the dN or a derivative thereof is deoxycytidine (dC) or a derivative thereof and/or thymidine (dT) or a derivative thereof, preferably wherein the dN or a derivative thereof is deoxycytidine (dC) or a derivative thereof.
27. The use according to any of paragraphs 22, 25 or 26, or the method according to any of paragraphs 23 to 26, wherein the dN or a derivative thereof is at a concentration of from about 25 μM to about 1000 μM, preferably wherein the dN or a derivative thereof is at a concentration of from about 100 μM to about 500 μM.
28. The use according to any of paragraphs 22, 25 to 27, or the method according to any of paragraphs 23 to 27, wherein the population of cells is contacted with the dN or a derivative thereof in combination with CsH or a derivative thereof.
29. The use according to paragraph 28, or the method according to paragraph 28, wherein the CsH or derivative thereof is at a concentration of from about 1 to about 50 μM.
30. The use according to any of paragraphs 25 to 29, or the method according to any of paragraphs 25 to 29, wherein the population of cells is further contacted with a purine dN or a derivative thereof.
31. The use according to paragraph 30, or the method according to paragraph 30, wherein the purine dN or a derivative thereof is at a concentration of from about 25 μM to about 1000 μM, preferably wherein the purine dN or a derivative thereof is at a concentration of from about 100 μM to about 500 μM.
32. The use according to any of paragraphs 19, 22, 25 to 31, or the method according to any of paragraphs 20, 21, 23 to 31, wherein the cells are human cells or mouse cells, preferably human cells.
33. The use according to any of paragraphs 19, 22, 25 to 32, or the method according to any of paragraphs 20, 21, 23 to 32, wherein the cells are quiescent cells.
34. The use according to any of paragraphs 19, 22, 25 to 33, or the method according to paragraph 20 or 21, wherein the population of cells comprises or consists substantially of:
   (i) haematopoietic stem and/or progenitor cells (HSPCs); and/or
   (ii) CD14⁻ peripheral blood mononuclear cells (PBMCs).
35. The use according to paragraph 34, or the method according to any of paragraphs 23 to 34, wherein the HSPCs are unstimulated HSPCs.
36. The use according to paragraph 34 or 35, or the method according to any of paragraphs 23 to 35, wherein the HSPCs are CD34⁺ or CD34⁻ cells, preferably wherein the CD34⁺ cells are CD34⁺CD133⁻CD90⁻, CD34⁺CD133⁺CD90⁻, or CD34⁺CD133⁺CD90⁺ cells, more preferably wherein the CD34⁺ cells are CD34⁺CD133⁺CD90⁺ cells.
37. The use according to any of paragraphs 34 to 36, or the method according to any of paragraphs 23 to 36, wherein the PBMCs are CD3⁺, CD4⁺, and/or CD8⁺ T cells.
38. The use according to any of paragraphs 34 to 37, or the method according to any of paragraphs 23 to 37, wherein the method further comprises a step of enriching the population of cells for the HSPCs or CD14⁻ PBMCs.
39. The use according to any of paragraphs 19, 22, 25 to 38, or the method according to any of paragraphs 20, 21, 23 to 38, wherein the viral vector is a retroviral vector, preferably a lentiviral vector.
40. The use according to paragraph 39, or the method according to paragraph 39, wherein the lentiviral vector is an integration-defective lentiviral vector.
41. The use according to any of paragraphs 19, 22, 25 to 40, or the method according to any of paragraphs 20, 21, 23 to 40, wherein the percentage of cells transduced by the vector is increased and/or the vector copy number per cell is increased.
42 A method of gene therapy comprising the steps of:
   (a) transducing a population of cells according to the method of any of paragraphs 20, 21, 23 to 41; and
   (b) administering the transduced cells to a subject.
43. The method of paragraph 42, wherein the transduced cells are administered to a subject as part of an autologous stem cell transplant procedure or an allogeneic stem cell transplant procedure.
44 A population of cells prepared according to the method of any of paragraphs 20, 21, 23 to 41.
45. A pharmaceutical composition comprising the population of cells of paragraph 44.
46. The population of cells of paragraph 44 for use in therapy.
47. The population of cells for use according to paragraph 46, wherein the population is administered as part of an autologous stem cell transplant procedure or an allogeneic stem cell transplant procedure.
48. The combination according to any of paragraphs 1 to 16, the composition according to paragraph 17, or the kit according to paragraph 18, for use in gene or cell therapy.
49. A deoxyribonucleoside (dN) or a derivative thereof for use in gene or cell therapy.

### EXAMPLES

The invention will now be further described by way of examples, which are meant to serve to assist one of ordinary skill in the art in carrying out the invention and are not intended in any way to limit the scope of the invention.

### Example 1—Gene Engineering of Quiescent Human Hematopoietic Stem Cells

Exogenous Deoxynucleosides Synergize with CsH to Significantly Improve Lentiviral Transduction and Gene Editing in Quiescent HSPC

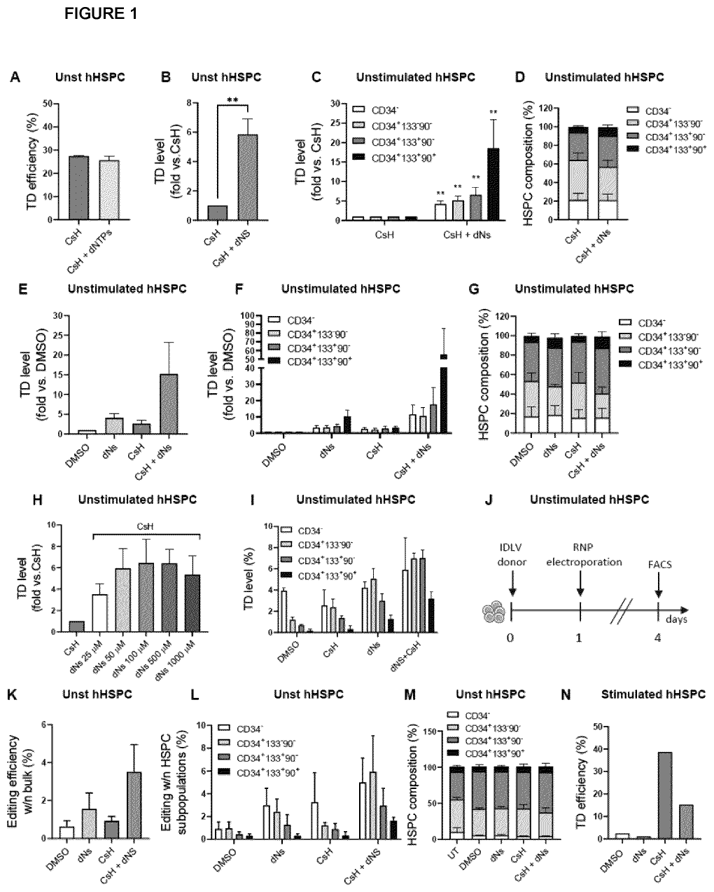

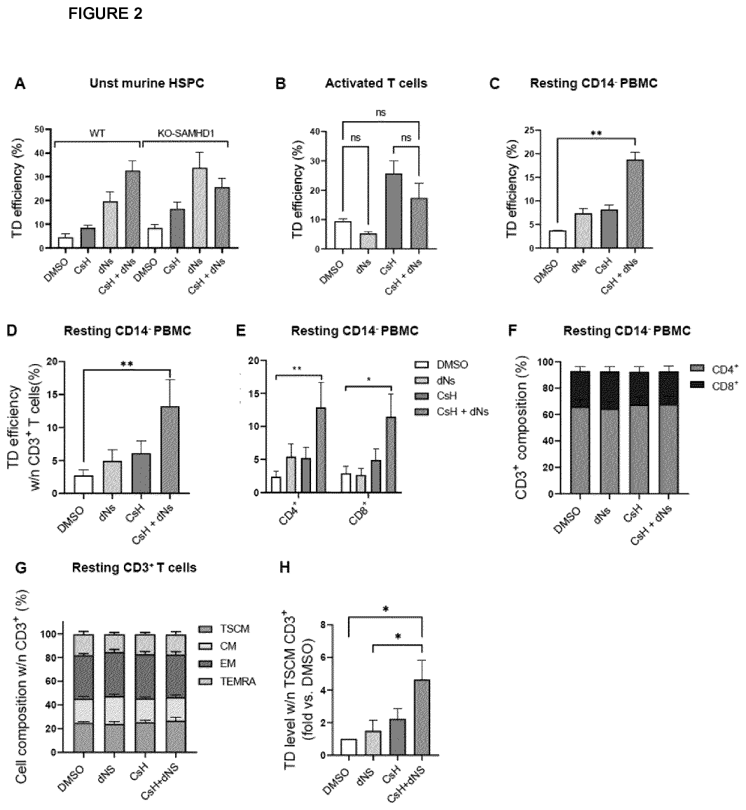

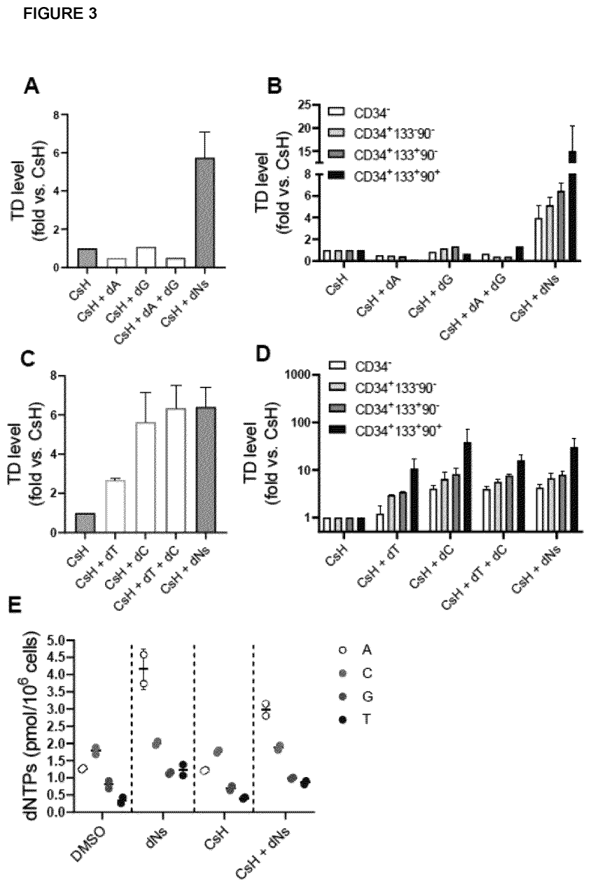

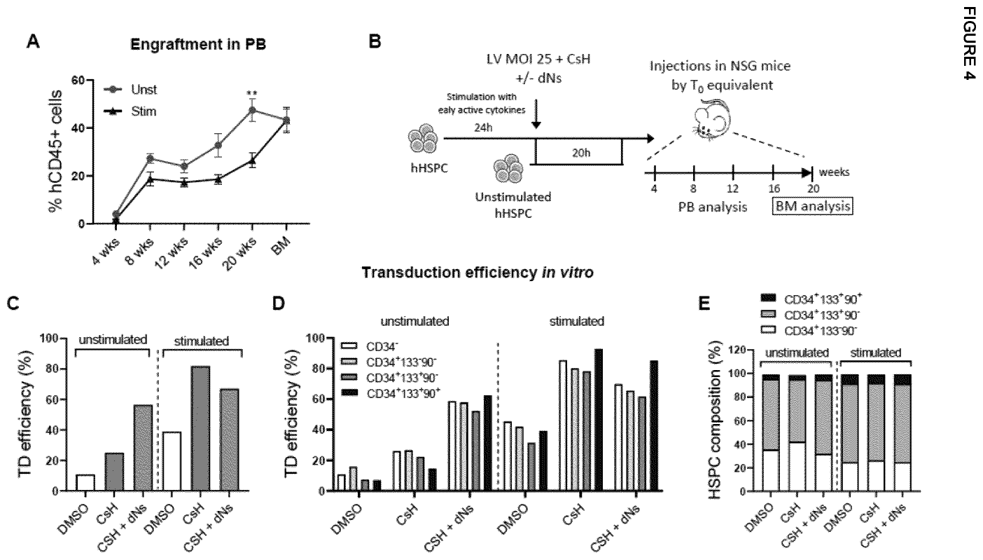

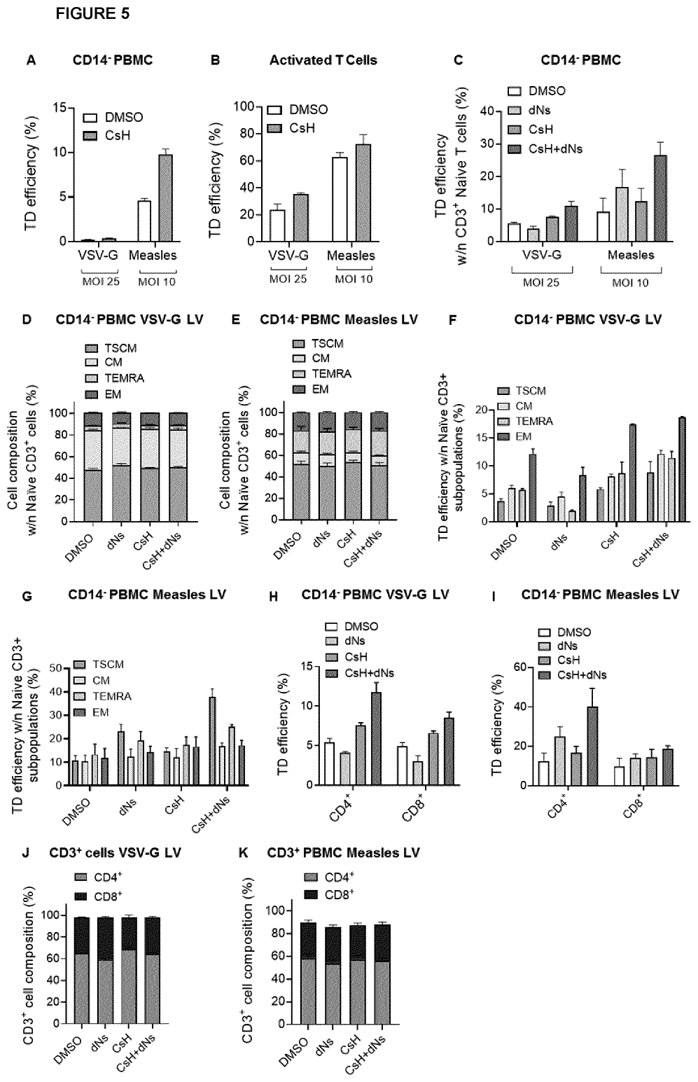

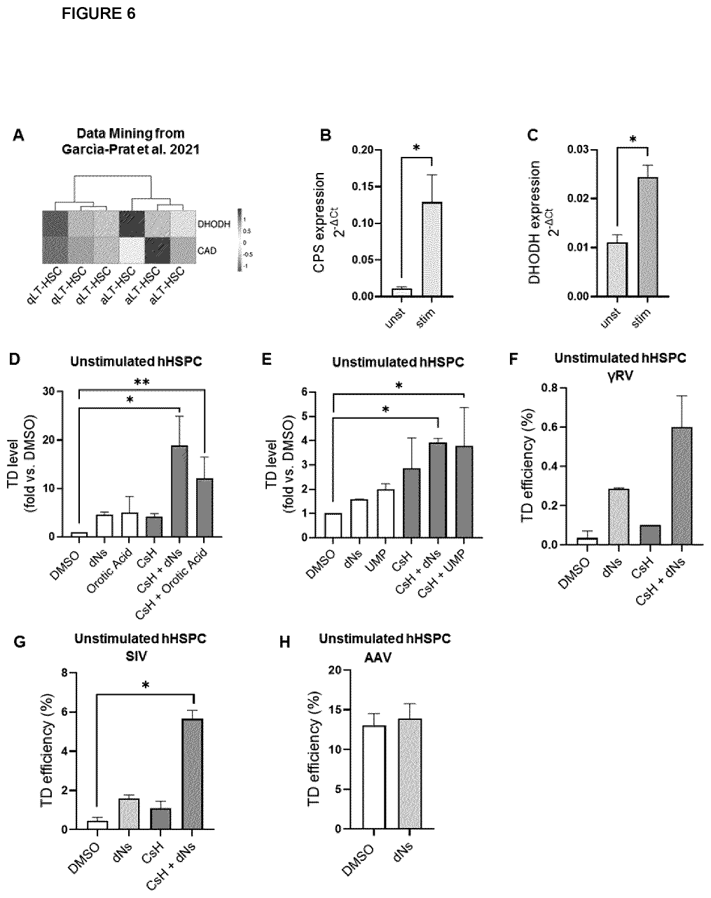

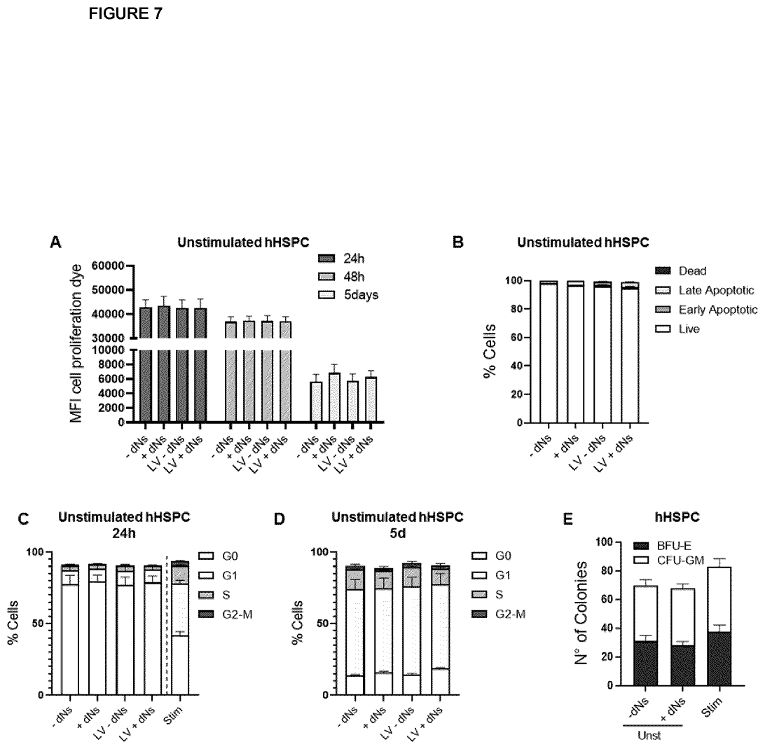

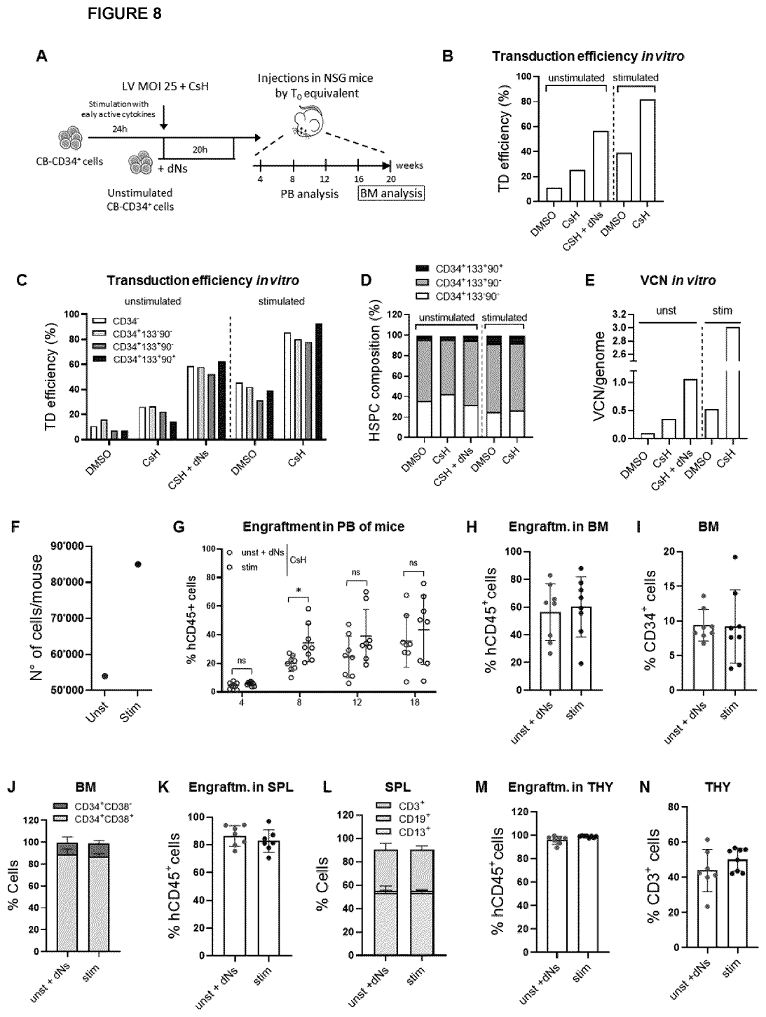

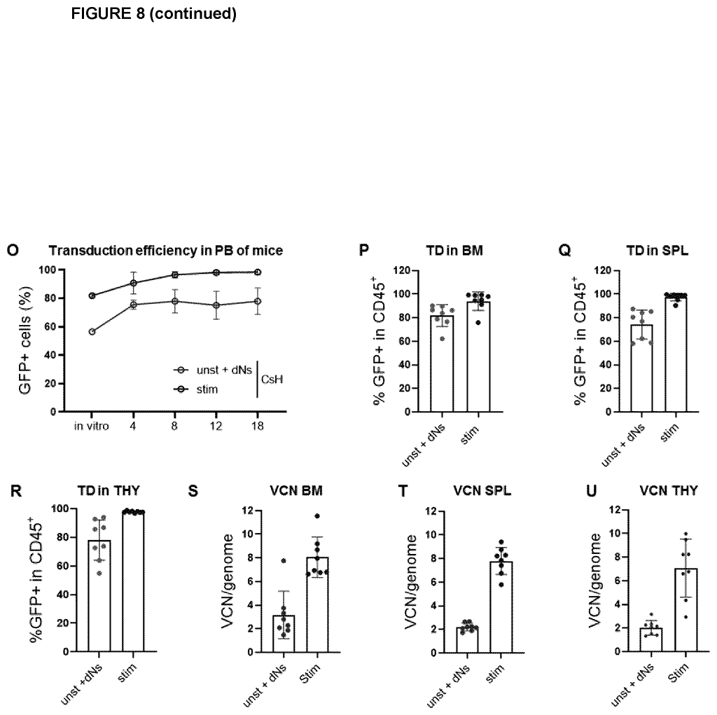

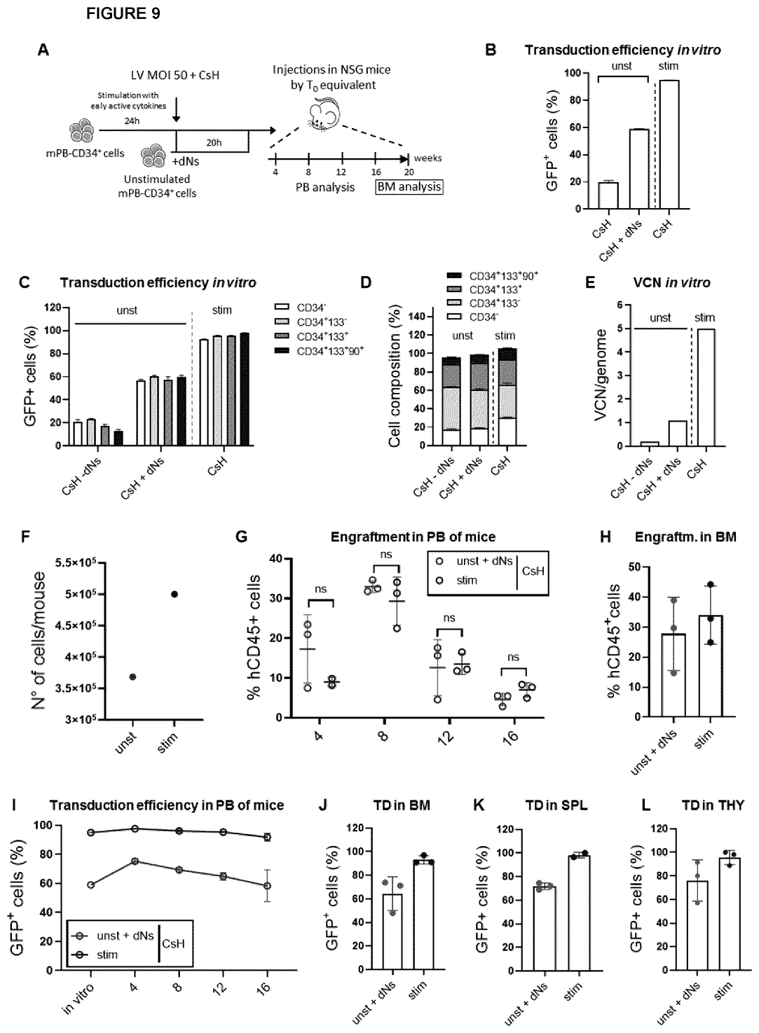

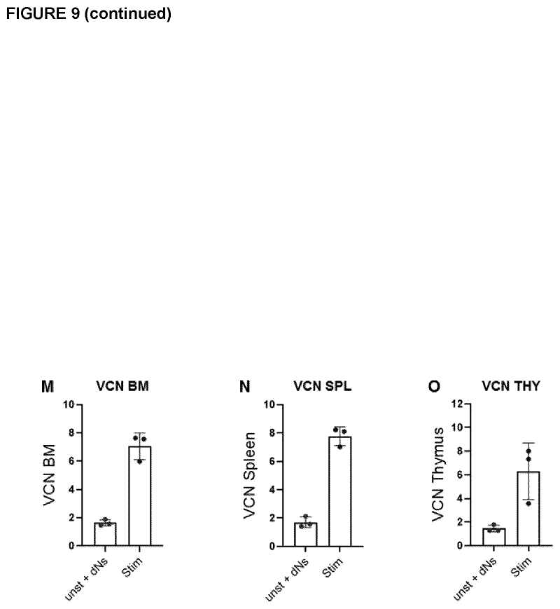

We evaluated the impact of an excess of nucleotides on gene transfer efficacy. We observed that deoxynucleotide triphosphate (dNTP) addition had no effect on the transduction efficiency of unstimulated HSPC, even in combination with CsH (FIG. 1A).

Subsequently, we provided deoxynucleosides (dNs), precursors of dNTPs, during transduction. Remarkably, a striking effect was observed when CsH was combined with addition of dNs during transduction of quiescent HSPC, yielding up to 6-fold increase in the percentage of GFP⁺ cells over the CsH alone control condition (FIG. 1B).

This benefit was significant in all fractions of HSPC with the strongest effect observed in the most primitive CD34⁺CD133⁺CD90⁺ compartment, resulting in an average 18-fold increase in transduction over CsH alone (FIG. 1C). No major impact on subpopulation composition was observed upon dNs addition (FIG. 1D).

Remarkably, dNs alone rendered HSPC as permissive as CsH alone, leading to similar increase in transduction among the different HSPC subpopulations without affecting their composition (FIG. 1E-G).

Of note, dNs retained their ability to enhance transduction in unstimulated HSPC over a dose range of 25-1000 μM, with higher or lower concentration still improving transduction but to a lesser extent as compared to the intermediate 100 and 500 μM doses (FIG. 1H).

dNs addition also enhanced transduction of an integrase-defective LV (IDLV) (FIG. 1I). We therefore tested effect of dNs addition on the efficiency of IDLV-mediated HSPC gene editing. As expected, despite overall low efficiency due to the quiescent status of these cells, combination of CsH and dNs enhanced targeted genome editing in unstimulated HSPC, without altering the relative composition of the hematopoietic subpopulations (FIG. 1J-M). Adding dNs did not benefit transduction in pre-stimulated HSPC, even in combination with CsH (FIG. 1N), suggesting that the intracellular dNTP pools of stimulated HSPC are not limiting for viral reverse transcription.

### Combination of CsH and Exogenous Deoxynucleosides Improves Transduction Across Species and in Multiple Quiescent Hematopoietic Cell Types

To test whether the combination of CsH and dNs leads to increased transduction across species, we tested and confirmed dNs-mediated increase in transduction also in unstimulated murine HSPC. Although a slightly higher basal level of transduction was observed in murine HSPC from SAMHD1 KO mice (FIG. 2A), the effect of dNs on the increase in transduction was irrespective of SAMHD1-mediated LV restriction (FIG. 2A), suggesting that the intracellular dNTP pools may be a limiting factor also in the murine context and that lack of SAMHD1 is not sufficient to maximally remove this restriction.

Additionally, while activated CD3⁺ T cells did not benefit of dNs addition (FIG. 2B), a significant increase in gene transfer efficacy was observed in quiescent CD14⁻ PBMC in combination with CsH (FIG. 2C). Importantly this effect was maintained within the CD3⁺ T cell compartment and the CD4⁺ and CD8⁺ subsets (FIG. 2D-E), where no major impact on cellular composition was observed upon dNs addition (FIG. 2F-G). Importantly, the dNs and CsH combination significantly improved LV transduction also in the quiescent Stem memory T cells (TSCM) (FIG. 2H). Taken together, these data suggest that addition of exogenous nucleosides together with CsH-mediated removal of IFITM3 restriction will allow efficient gene modification of difficult to transduce quiescent targets of gene therapy such as resting T cells.

### Exogenous Pyrimidines Mediate Lentiviral Transduction Enhancement in Quiescent HSPC

Quiescent HSPC were transduced in presence of CsH and the single nucleoside or the combination of purines (dA and dG) or pyrimidines (dC and dT). We did not observe any effect of the single addition of dA or dG or of the combination of the two purine dNTP precursors on the transduction efficiency of quiescent HSPC (FIG. 3A-B). Instead, this effect was mainly mediated by the addition of the two pyrimidines dNTP precursors, with dC alone leading to gene marking levels comparable to the combination of all dNs (FIG. 3C-D).

To further dissect the mechanism behind pyrimidine-mediated improvement of transduction in quiescent cells we evaluated how addition of exogenous dNs to the culture media impacted on the intracellular pools of single dNTPs. Surprisingly, dNs addition to quiescent HSPC did not lead to homogeneous increase in all dNTP levels but impacted mainly deoxyadenosine triphostphate (dATP) and deoxythymidine triphosphate (dTTP) levels with little or no effect on deoxyguanosine triphosphate (dGTP) and deoxycytidine triphosphate (dCTP) levels (FIG. 3E). Of note, CsH did not alter intracellular dNTP pools in quiescent HSPC (FIG. 3E). Taken together these results suggest that pyrimidines are the limiting dNTPs in quiescent HSPC.

### Unstimulated HSPC Engraft to Higher Extend Despite Lower Cell Input as Compared to Pre-stimulated Counterparts

Ex-vivo culture of HSPC impacts on their engraftment potential, due to cell cycle progression that drives lineage commitment and differentiation, as well as loss of adhesion molecules, which impact on their homing capacity into the bone marrow niche (Glimm H, et al. (2000) Blood 96: 4185-4193). We confirmed these observations in our experimental settings as unstimulated HSPC yielded higher peripheral graft output in the immunocompromised NSG mice as compared to stimulated HSPC despite lower initial cellular input (FIG. 4A).

We reasoned that an efficient gene transfer into quiescent HSPC should allow better preservation of their biological capacities, including long-term repopulation capacity. Therefore, experiments assessing in vivo engraftment capacity of human quiescent HSPC transduced with LV in presence of CsH, with or without addition of dNs, into the xenograft NSG mouse model of human hematopoiesis are ongoing (FIG. 4B). As a control, HSPC were pre-stimulated over/night with human early-acting cytokines and transduced under the same conditions of their unstimulated counterpart (FIG. 4B). Importantly, the in vitro analysis of these experiments shows that our combinatorial strategy in unstimulated HSPC yielded higher gene marking compared to control transduced stimulated HSPC, reaching 60% of GFP⁺ cells at the moderate multiplicity of infection (MOI) of 25, while no advantage of dNs addition was observe in stimulated HSPC (FIG. 4C-D). No major impact on subpopulation composition was observed (FIG. 4E).

### Conclusion

Quiescent resting HSPC and T cells would be attractive targets for cell and gene therapies due to their higher and more stable long-term engraftment capacities over ex-vivo cultured counterparts (Pampusch M S, et al (2020) Mol Ther Methods Clin Dev 16: 1-10). Nevertheless, their genetic engineering remains a challenge in the field as they are highly refractory to viral vector mediated gene transfer and constraints inherent to their quiescent cell cycle status impair efficient homology directed repair (HDR) (Agosto L M, et al. (2009) J Virol 83: 8153-8162). Few studies have addressed poor permissiveness of quiescent HSCP or T cells and with limited success. The overall transduction levels remained well below the levels required for clinical translation.

We have developed here a novel lentiviral transduction protocol that allows efficient gene manipulation of quiescent unmanipulated human primary HSPC and resting T cells. We show that exogenously supplied dNs significantly improve LV transduction to similar extent as CsH in unstimulated human HSPC. Importantly, the combination of dNs with CsH led to a potent synergistic enhancement of LV transduction. In agreement, dNs improved LV transduction independently of SAMHD1 in the context of quiescent murine HSPC. Interestingly, measurement of intracellular levels of dNTPs in HSPC after exposure to exogenous dNs revealed no changes in dCTP levels despite dC being almost exclusively responsible for improving transduction. It is possible that nucleotide salvage pathway may be differently regulated in HSPC, explaining lack of increased dCTP levels upon exogenous dC administration but suggesting more complex nucleotide-independent mechanisms of transaction enhancement in this context.

Chimeric antigen receptor (CAR)-T cells show great promise in treating cancers and viral infections. However, most protocols developed to expand T cells require relatively long periods of time in culture, potentially leading to progression toward populations of terminally differentiated effector memory cells. Ideally, adoptively transferred T cells should express a less differentiated phenotype because those cell subsets circulate to lymphoid organs and are capable of robust expansion (Redeker A, Arens R (2016) Front Immunol 7: 345). In animal studies and human clinical trials, it is apparent that less differentiated T cells, defined as cells expressing the lymphoid homing molecules CD62L and CCR7, better contribute to engraftment and long-term persistence (Klebanoff C A, et al (2012) J Immunother 35: 651-660). In clinical trials, a failure of infused T cells to persist has been correlated with the absence of CD4⁺ T cells in the CD8⁺ T cell product (Patel S, et al (2016) Cytotherapy 18: 931-942).

The use of PBMCs in the production of the CAR-T cells in this protocol allows the final cultures to contain both antigen-specific CD8⁺ T cells and CD4⁺ T cells. For efficient viral gene transfer, PBMCs are activated with anti-CD3 and anti-CD28 along with interleukin 2 (IL-2) at a relatively high density. Moreover, CD4⁺ T cells are more refractory to lentiviral transduction as compared to CD8+ T cells. In this context, our CsH+dNs based lentiviral transduction protocol could offer the possibility to generate CAR-T cells from minimally manipulated PBMC as well as to achieve gene marking also in the relevant resting T cell populations including the T stem memory compartment (Gattinoni L, et al (2011) Nat Med 17: 1290-1297).

Overall, our results indicate that multiple innate immune barriers restrict gene transfer into quiescent HSPC and T cells and pave the way for the development of genetic engineering strategies directly in unstimulated targets of cell and gene therapies.

### Example 2—Materials and Methods

### Vectors

Third generation LV stocks were prepared, concentrated and titered as previously described (Dull T, et al. (1998) J Virol 72: 8463-8471; Follenzi A, et al. (2000) Nat Genet 25: 217-222). Briefly, self-inactivating (SIN) LV vectors were produced using the transfer vector pCCLsin.cPPT.hPGK.eGFP.Wpre, the packaging plasmid pMDLg/pRRE, Rev-expressing pCMV-Rev and the VSV-G envelop-encoding pMD2.VSV-G plasmids. IDLV was produced as previously described (Lombardo A, et al (2007) Nat Biotechnol 25: 1298-1306) substituting the packaging plasmid pMDLg/pRRE with pMD.Lg/pRRE.D64VInt.

### Mice

NOD.Cg-Prkdcscid II2rgtm1Wjl/SzJ (NSG, RRID:IMSR_ARC:NSG) Mus musculus were purchased from Jackson laboratory. All animal procedures were performed according to protocols approved by the Animal Care and Use Committee of the Ospedale San Raffaele (IACUC 782) and communicated to the Ministry of Health and local authorities according to the Italian law. Female 8-10 week old mice were used in all studies to allow better engraftment of human HSPC cells upon tail vein transplantation (Notta F, et al. (2010) Blood 115: 3704-3707). Mice were observed carefully by laboratory staff and veterinarian personnel for health and activity. Animals were monitored to ensure that food and fluid intake meets their nutritional needs. Mice were housed in cages with microisolator tops on ventilated or static racks in a specific pathogen-free facility. All caging materials and bedding are autoclaved. All mice were randomized into different HSC transplantation groups. On the basis of a standard backward sample size calculation, we transplanted at least three to ten mice per condition to obtain a sufficient number of mice to perform statistical analysis. Human cell engraftment was blindly assessed by serial bleeding or bone marrow as well as spleen analysis at sacrifice. At the end of the experiments mice were euthanized by CO2.

### Cells

### Cell Lines

The human embryonic kidney 293T cells (HEK293T, RRID:CVCL_1926) were maintained in Iscove's modified Dulbecco's medium (IMDM; Sigma). Medium was supplemented with 10% fetal bovine serum (FBS; GIBCO), penicillin (100 IU/ml), streptomycin (100 mg/ml) and 2% glutamine.

### Primary Cells

Human CD34⁺ HSPC and CD14⁺ monocytes were isolated through positive magnetic bead selection according to manufacturer's instructions (Miltenyi) from umbilical cord blood or from mononuclear cells collected upon informed consent from healthy volunteers according to the Institutional Ethical Committee approved protocol (TIGET01/09). Otherwise, cord blood (CB), bone marrow (BM) or G-CSF mobilized peripheral blood (mPB) CD34+ cells were directly purchased from Lonza or Hemacare. Monocyte-derived macrophages (MDM) were differentiated from isolated CD14⁺ monocytes in DMEM supplemented with 10% FBS, penicillin (100 IU/ml), streptomycin (100 mg/ml), 2% glutamine and 5% human serum AB (Lonza) for seven days. Primary T lymphocytes from healthy donors' PB mononuclear cells were isolated and activated using magnetic beads conjugated to anti-human CD3 and CD28 antibodies (Dynabeads human T-activator CD3/CD28; Invitrogen) in RPMI medium (GIBCO-BRL) supplemented with penicillin, streptomycin, glutamine, 10% FBS, and 5 ng/ml of IL-7 and IL-15 (PeproTech) for 3 days as described (Provasi E, et al (2012) Nat Med 18: 807-815).

Mononuclear cells collected upon informed consent from healthy volunteers according to the Institutional Ethical Committee approved protocol (TIGET01/09) were isolated and depleted of CD14+ for resting T cells culture. Cells were maintained in RPMI medium (GIBCO-BRL) supplemented with penicillin, streptomycin, glutamine, 10% FBS, non-essential amino acids (1 mM) (GIBCO-BRL) and sodium pyruvate (1 mM) (GIBCO-BRL). Frozen WT or KO SAMHD1 BM cells were kindly provided by Rayk Behrendt's group. HSPCs were purified by Lin-selection using the mouse Lineage Cell Depletion Kit (Miltenyi Biotec) according to the manufacturer's instructions. All cells were maintained in a 5% CO2 humidified atmosphere at 37 C.

### Compounds

Cyclosporine H (Sigma-Aldrich) was resuspended and stored following the manufacturer's instructions. Where indicated CsH was added to the transduction medium at 8 μM concentration and washed out with the vector 16-20 hours later. dNTPs (NEB N1201AA) were added to the transduction medium where indicated at 10 uM concentration and washed out with the vector 16-20 hours later. dA (D8668), dC (D0776), dG (D0901) and dT (T1895) were all purchased from Sigma-Aldrich and resuspended and stored following the manufacturer's instructions. dNs mix, single dNs or combinations of dNs (500-1000 μM each) were added to the culture media 4 h-2 h before transduction and washed out with the vector 16-20 hours later. For dosage experiment 25 μM to 1000 μM concentrations of each dN was tested.

### Transduction

All cells were transduced at the indicated multiplicity of infection (MOI) as calculated by titration of vector stocks on 293T cells and expressed as transducing units (TU)/293T cell. For transduction, CB-derived HSPC were cultured in serum-free StemSpan medium supplemented with penicillin (100 IU/ml), streptomycin (100 mg/ml), 100 ng/ml recombinant human stem cell factor (rhSCF), 20 ng/ml recombinant human thrombopoietin (rhTPO), 100 ng/ml recombinant human Flt3 ligand (rhFlt3), and 20 ng/ml recombinant human IL6 (rhIL6) (all from Peprotech) 16 to 24 hours prior to transduction. Bone marrow and G-CSF mobilized peripheral blood CD34+ cells were cultured in CellGro medium (Cell Genix) supplemented with penicillin (100 IU/ml), streptomycin (100 mg/ml) and a cocktail of cytokines: 60 ng/ml IL-3, 100 ng/ml TPO, 300 ng/ml SCF, and 300 ng/ml FLT-3L (all from Cell Peprotech). HSPC were then transduced at a concentration of 1×10⁶ cells per milliliter with the vector for 16 hours at the indicated multiplicity of infection (MOI), expressed as transducing units (TU)/293T cell, in the same medium. After transduction cells were washed and maintained in serum-free medium supplemented with cytokines as above until the reading of the percentage of positive cells by FACS, after which they were maintained in IMDM supplemented with 10% FBS, 25 ng/ml rhSCF, 5 ng/ml rhIL6-3, 25 ng/ml rhFlt3 and 5 ng/ml rhTPO. Unstimulated HSPC were transduced freshly isolated in StemSpan or CellGro medium supplemented with penicillin (100 IU/ml), streptomycin (100 mg/ml) for 16-24 hours and then maintained in presence of human cytokines and 10 mM of the reverse-transcriptase inhibitor 3TC (from SIGMA) to avoid subsequent transduction due to cytokines stimulation. Unstimulated murine Lin-HSPC were transduced freshly isolated in serum-free StemSpan medium (StemCell Technologies) containing penicillin, streptomycin and glutamine at a concentration of 1×10⁶ cells/ml for 16-20 hours and then maintained in presence of a combination of murine cytokines (20 ng/ml IL-3, 100 ng/ml SCF, 100 ng/ml Flt-3L, 50 ng/ml TPO all from Peprotech). MDM were transduced 7-10 days after differentiation. Activated T lymphocytes were transduced at a concentration of 10⁶ cells/ml, after 3 days of stimulation with Dynabeads human T-activator CD3/CD28. Total CD14⁻ PBMC were transduced fresh at a concentration of 10⁶ cells/ml for measurement of transduction.

### Gene Editing of Human Cells

For gene editing experiments in unstimulated human HSPC, cells were immediately transduced with IDLV at MOI 100, in presence or not of CsH and dNs. IDLV donor was generated using HIV-derived, third-generation self-inactivating transfer construct and the IDLV stock was prepared by transient transfection of HEK293T, as previously described (Petrillo C, et al (2018) Cell Stem Cell 23: 820-832 e829). At 30 hours post-transfection, vector-containing supernatant was collected, filtered, clarified, DNAse treated and loaded on a DEAE-packed column for Anion Exchange Chromatography. The vector-containing peak was collected, subjected to a second round of DNAse treatment, concentration by Tangential Flow Filtration and a final Size Exclusion Chromatography separation followed by sterilizing filtration and titration of the purified stock as previously described (Petrillo C, et al (2018) Cell Stem Cell 23: 820-832 e829). IDLV donor template with homologies for AAVS1 locus (encoding for a PGK.GFP reporter cassette (Genovese P, et al (2014) Nature 510: 235-240) was utilized. After 24 hours from IDLV transduction, cells were washed with PBS and electroporated (P3 Primary Cell 4D-Nucleofector X Kit, program EO-100; Lonza) with 1.25 mM of ribonucleoproteins (RNP). RNPs were assembled by incubating at 1:1.5 molar ratio Streptococcus pyogenes (Sp)Cas9 protein (Aldevron) with synthetic cr:tracrRNA (Integrated DNA Technologies) for 10′ at 25C. Electroporation enhancer (Integrated DNA Technologies) was added prior to electroporation according to manufacturer's instructions. Genomic sequence recognized by the gRNA for AAVS1 locus is the following: TCACCAATCCTGTCCCTAGtgg. After electroporation cells were maintained in presence of human cytokines and 10 mM of the reverse-transcriptase inhibitor 3TC. For AAVS1 edited cells, editing by homology-directed repair (HDR) was quantified by flow cytometry measuring the percentage of cells expressing the GFP marker, 3 days after electroporation.

dNTP Measurements

Measurement was done as previously described (Diamond T L, et al (2004) J Biol Chem 279: 51545-51553). Cells were lysed with 60% cold methanol. Samples were vortexed, heated at 95° C. for 3 min and cellular debris were cleared by 14 K rpm centrifugation. Supernatant was dried using a SpeedVac. Pellets were resuspended in water diluted to be within linear range of the assay, 2-50%. 5′ 32P-end-labeled 18-mer DNA primer (5′-GTCCCTCTTCGGGCGCCA-3′, Integrated DNA Technologies) was individually annealed to one of four unique 19-mer DNA templates (3′-CAGGGAGAAGCCCGCGGTN-5′, Integrated DNA Technologies). Reactions contained 200 fmol template/primer, 4 μL of purified RT (HIV-1 HXB2), 25 mM Tris-HCl, pH 8.0, 2 mM dithiothreitol, 100 mM KCl, 5 mM MgCl2, and 10 μM oligo (dT), and cellular dNTP extracts with a final reaction volume of 20 μL. After 5 min incubation at 37° C., each reaction was stopped with 10 μL of 40 mM EDTA and 99% (vol/vol) formamide at 95° C. for 2 min. The reaction causes the template/primer to be extended by HIV-1 reverse transcriptase, generating one additional nucleotide extension product for one of four dNTPs contained in the sample. These products are resolved on a 14% urea-PAGE gel (AmericanBio, Inc.) and analyzed using PharosFX molecular imager and Image lab software (Biorad). In this assay, the molar amount of product is equal to that of each dNTP contained in the extracted samples, which allows us to calculate and compare the dNTP concentrations for different dN treatments (Diamond T L, et al (2004) J Biol Chem 279: 51545-51553).

RNA Extraction, qPCR and Gene Expression Analysis

RNA extraction from cells was performed using the RNeasy Plus micro Kit (QIAGEN). Briefly, cells were lysed in Buffer RLT plus, supplemented with beta-mercaptoethanol. RNA was then extracted according to manufacturer's instructions. The extracted mRNAs were reverse transcribed (RT) using SuperScript™ IV VILO™ Master Mix (11766050, Invitrogen). RT-qPCR analyses were performed using TaqMan probes from Applied Biosystems to detect endogenous mRNA levels. Q-PCR was run for 40 cycles using the Viia 7 instrument while the Viia 7 software was then used to extract the raw data (Ct). To determine gene expression, the difference (DCt) between the threshold cycle (Ct) of each gene and that of the reference gene was calculated by applying an equal threshold. Relative quantification values were calculated as the fold-change expression of the gene of interest over its expression in the reference sample, by the formula 2-DDCt. The expression was normalized using the housekeeping gene HPRT1. The following Taqman probes from Applied Biosystems were used: P21 (Hs00355782_m1), ISG-15 (Hs01921425_s1), HPRT1 (Hs01003267_m1).

### Transplantation of Human HSPC in NSG Mice

Human HSPC were kept unstimulated or pre-stimulated for 24 h and transduced with a LV at an MOI of 25 in presence or not of CsH and dNs. After transduction cells were infused into the tail vein of sublethally irradiated 8-10 week-old NSG mice (radiation dose: 200 cGy for mice weighting 18-25 g and of 220 cGy for mice above 25 g of weight). Transduced and untransduced cells were also cultured in vitro for 14 days for further analysis. Peripheral blood was sampled at indicated times post-transplant and analyzed. At 20 weeks all mice were sacrificed by CO2 to analyze and BM, spleen and thymus were collected and analyzed.

### Flow Cytometry

All cytometric analyses were performed using the FACS Canto III instrument and analyzed with the FACS Express software (De Novo Software).

### Transduced Cells

GFP expression in transduced cells was measured 3-5 days post-transduction. Adherent MDM were detached by Tripsin-EDTA, washed and resuspended in PBS containing 2% fetal bovine serum (FBS). Cells grown in suspension were washed and resuspended in PBS containing 2% FBS. To measure HSPC subpopulation composition cells were harvested and incubated with anti-human receptor blocking antibodies for 15 min at 4° C. and then stained for 20 min at 4° C. with anti-human CD34 (RRID:AB_10827793), CD133 (RRID:AB_244346), CD90 (RRID:AB_559869) antibodies. In order to measure T cells subsets cells were harvested and incubated with anti-human receptor blocking antibodies for 15 min at 4° C. and then stained for 20 min at 4° C. with anti-human CD3 (RRID:AB_300328), CD4 (RRID:AB_558116; RRID:AB_317416), CD8 (RRID:AB_641400; RRID:AB_344712), CD45RA (RRID:AB_550855, RRID:AB_130-095-464) and CD62L (RRID:AB_555544, RRID:AB_304822). To exclude dead cells from the analysis, 10 ng/ml 7-aminoactinomycin D (7-AAD) was added.

Peripheral Blood from Mice

For each mouse, 250 ml of peripheral blood were added to 15 mL of PBS containing 45 mg/ml EDTA. For immunostaining a known volume of whole blood (100 ml) was first incubated with anti-human FcR Blocking Reagent and anti-mouse FcgIII/II receptor (Cd16/Cd32) blocking antibodies for 15 min at 4° C. and then incubated in the presence of anti-human CD45 (RRID:AB_1944368), CD19 (RRID:AB_345789), CD13 (RRID:AB_562596), CD3 (RRID:AB_398591) for 20 min at 4° C. Erythrocytes were removed by lyses with the TQ-Prep workstation (Beckman-Coulter) in the presence of an equal volume of FBS (100 ml) to protect white blood cells.

### Bone Marrow

BM cells were obtained by flushing the femurs in PBS 2% FBS solution. Cells (1×10⁶ cells) were washed, resuspended in 100 mL of PBS containing 2% FBS, and incubated with anti-human receptor (Cd16/Cd32) blocking antibodies for 15 min at 4 C. Staining was then performed with anti-human CD45, CD19, CD33 for 20 min at 4 C. Cells were washed and finally resuspended in PBS containing 2% FBS.

### Spleen

Spleens were first smashed and the resulting cell suspension was passed through 40 mm nylon filter and washed in cold phosphate buffered saline (PBS) containing 2 mM EDTA and 0.5% bovine serum albumine (BSA). Cells were incubated with anti-human receptor (Cd16/Cd32) blocking antibodies for 15 min at 4 C and then stained with anti-human CD45, CD19, CD13, CD3 for 20 min at 4 C. Cells were finally washed and resuspended in PBS containing 2% FBS.

### Quantification and Statistical Analysis

All statistical analyses were conducted with GraphPad Prism 9.0 version. In all studies, values are expressed as mean±standard error of the mean (SEM) and all n numbers represent biological repeats. Statistical analyses were performed by Mann Whitney or Wilcoxon Signed Rank test between means of two groups and by Dunn's adjusted Kruskal-Wallis for multiple comparisons, as indicated in the Figure legends. Differences were considered statistically significant at *p<0.05, **p<0.01, ***p<0.001, ****p<0.0001, “ns” represents non significance.

All publications mentioned in the above specification are herein incorporated by reference. Various modifications and variations of the disclosed agents, methods, cells, compositions and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

### Example 3—Further Gene Engineering of Quiescent Human Hematopoietic Stem Cells

Measles Pseudotyped LV Outperforms the Gold Standard VSV-G LV Significantly Increasing Transduction Yields in Resting CD14⁻ PBMC Pretreated with dNs and CsH

We previously showed that the combination of CsH and dNs significantly increases gene transfer efficacy in quiescent CD14⁻ PBMC. In an attempt to further implement transduction yields in this poorly permissive hematopoietic subset we tested Measles pseudotyped LV. Interestingly, higher transduction efficiencies were achieved using lower doses of Measles-LV as compared to higher doses of the clinical standard VSV-G pseudotyped lentiviral vectors in the same donor of resting CD14⁻ PBMC or activated T cells (FIG. 5A-B). Remarkably, the combination of dNs+CsH and Measles-LV further boosted the level of gene marking in resting CD14⁻ PBMC outperforming our formerly reported protocol with VSV-G LV (FIG. 5C). Importantly, no relevant differences were observed at the level of cell composition between the two transduction protocols despite almost four-fold higher levels of transduction were observed within the most relevant to target Stem memory T cell compartment when using Measles-LV in combination to dNs and CsH (FIG. 5D-E). Increased transduction levels with Measles-LV were measured also within CD4⁺ and CD8⁺ T cells (FIG. 5H-I) with no major impact on the percentages of cell composition (FIG. 5J-K). Overall, our findings show that it is possible to target resting T cells achieving good levels of gene marking within all T cell subsets but most importantly within the Stem memory compartment.

### Pyrimidine Pools are Limiting for Lentiviral Transduction in Quiescent HSPC

Given the lack of dCTP increase upon addition of dNs to quiescent HSPC we further investigated the role of pyrimidines to exclude potential dNTP-independent effects of exogenous dNs addition on LV transduction. We first exploited publicly available datasets to look at the endogenous levels of the two rate limiting enzymes of the ‘the novo pyrimidine synthesis’ pathway, in quiescent versus activated HSPC. As expected from low metabolically active cells, Carbamoyl-P Synthetase (CPS) and Dihydroorotate Dehydrogenase (DHODH) were downregulated in quiescent long-term repopulating HSC (qLT-HSCs) as compared to their activated counterparts (FIG. 6A). Importantly, these data were validated by gene expression analysis in stimulated and unstimulated HSPC derived from the same donors (FIG. 6B-C).

We thus hypothesize that fuelling the pathway with downstream precursors of the pyrimidines synthesis could have similar effect of providing exogenous dC and dT. Interestingly, orotic acid (OA) addition or uridine 5′-monophosphate (UMP) addition lead to an increase in LV transduction, similarly to dNs alone, which was further significantly increased when the two compounds were combined with CsH (FIG. 6D-E). Moreover, we confirmed the effect of dNs on transduction as specific for vectors that undergo a reverse transcription step. Indeed, while dNs alone or in combination with CsH increased transduction of unstimulated human HSPC with γRV (FIG. 6F) or SIV (FIG. 6G) vectors, no effect was observed when dNs were added during transduction with AAV (FIG. 6H).

Overall, these findings demonstrated that dNs addition enhances LV transduction in unstimulated HSPC by directly increasing the intracellular pools of pyrimidines that are required for efficient viral reverse transcription.

dNs Delivery does not Affect the Proliferation, Apoptosis and Cell Cycle Status of Unstimulated hHSPC

To further determine the impact of dNs on the HSC function, we performed in vitro assays to evaluate potential effects of dNs on proliferation, apoptosis or cell cycle status of unstimulated HSPC. Unstimulated HSPC were stained immediately after thawing with a fluorescent dye to monitor cell division. Then dNs were delivered to transduced or untransduced cells and the levels of cell proliferation dye at different time points was compared to that of cells that did not receive dNs. Importantly dNs addition did not cause any proliferation change, at any of the time point analysed, with LV transduction having no impact on the proliferation rate of unstimulated HSPC (FIG. 7A). Importantly, dNs turned out not to be toxic, as no signs of apoptosis were detected in unstimulated HSPC 48 h after dNs exposure both in transduced and untransduced cells (FIG. 7B).

As maintenance of HSPC quiescence could contribute to preserve their stem-cell properties, we evaluated whether dNs could alter the quiescent cell-cycle status of unstimulated HSPC. As expected, at 24 h unstimulated cells showed a greater percentage of cells in the G0 phase of the cell cycle in respect to their stimulated counterpart, with almost complete absence of cycling cells (FIG. 7C). Importantly, dNs delivery did not alter the quiescence status of these cells, neither at 24 h (FIG. 7C) nor at longer time points (FIG. 7D) both in transduced and untransduced conditions, suggesting that their stem-cell properties are preserved upon dNs addition.

We next investigated whether dNs affected the clonogenic potential of hematopoietic stem cells. Unstimulated HSPC showed a slightly lower colony output than stimulated HSPC, likely reflecting the lower percentage of progenitor cells due to the lack ex vivo culture (FIG. 7E). Importantly no difference in colony-forming capacity was observed between unstimulated cells that received dNs and control cells (FIG. 7E).

Overall, our data highlight a safe profile for dNs addition in unstimulated HSPC, prompting us to consider dNs as a valid option to reach good levels of transduction while preserving the quiescence and stem-cell properties of HSPC, potentially promoting higher engraftment in vivo.

### Unstimulated HSPC Engraft Similarly to Their Pre-stimulated Counterpart, Despite Lower Cellular Input

Ex-vivo culture of HSPC impacts on their engraftment potential, due to cell cycle progression that drives lineage commitment and differentiation, as well as loss of adhesion molecules, which impact on their homing capacity into the bone marrow niche. On these premises, we reasoned that an efficient gene transfer into quiescent HSPC should allow better preservation of their biological capacities, including long-term repopulation capacity. Therefore, we decided to directly assess the HSC engraftment potential of unstimulated cells transduced with our combinatorial transduction protocol, using gold-standard xenograft assay.

Unstimulated human Cord Blood (CB)-derived HSPC were transduced with LV in presence of CsH and dNs. As a control, HSPC were pre-stimulated overnight with human early-acting cytokines and transduced in presence of CsH alone. Cells were then transplanted into NSG mice by t₀ equivalent and engraftment and transduction efficiency were followed in vivo over time. Additional control conditions were kept for the in vitro analysis (FIG. 8A). Importantly, the in vitro data of these experiments show that our combinatorial strategy in unstimulated HSPC yielded higher gene marking compared to stimulated HSPC transduced in presence of DMSO alone, reaching 60% of GFP⁺ cells at the moderate multiplicity of infection (MOI) of 25 (FIG. 8B-C), with no major impact observed on subpopulation composition (FIG. 8D). The transduction increase was confirmed in terms of integrated viral copies, with CsH and dNs in unstimulated HSPC leading to more than twice the copies of DMSO-stimulated HSPC (FIG. 8E). Overall integrated copies in unstimulated cells remained lower compared to CsH-stimulated HSPC (FIG. 8E), in line with the expected strong enhancement mediated by CsH in these cells. Despite lower initial cellular input (FIG. 8F), unstimulated HSPC showed similar engraftment compared to their stimulated counterpart at longer time points (FIG. 8G). The analysis of the bone marrow at 20 weeks post-transplantation reflected the levels of human cells in the periphery and confirmed similar engraftment levels between unstimulated and stimulated HSPC (FIG. 8H). Moreover, similar percentages of human CD34⁺ cells were observed in the bone marrow between the two groups (FIG. 8I), with almost equal frequency of the more primitive CD34⁺ CD38⁻ and more committed CD34⁺ CD38⁺ fractions (FIG. 8J). Similar engraftment between the two groups was also confirmed in other hematopoietic organs (FIG. 8K-M), with no differences in lineage composition observed in the spleen of primary recipients (FIG. 8L) and similar frequency of human CD3⁺ cells retrieved from the thymus (FIG. 8N). Remarkably, the high transduction levels achieved with the combination of dNs and CsH in unstimulated cells were maintained in vivo and remained stable among all the time points analysed (FIG. 8O). The gene marking levels were also confirmed in the different organs at 20 weeks post-transplantation (FIG. 8P-R). While the difference in transduction between unstimulated and stimulated HSPC was not strong in terms of transgene expression, the number of integrated copies in vivo remained significantly lower in the unstimulated group (FIG. 8S-U). However, we obtained around 2-3 copies in vivo in the non-stimulated group, which should be sufficient to reach physiological expression level of a transgene, especially for pathologies in which supraphysiological levels are not required or even detrimental and is well within a safe and efficient target copy range for most gene therapy applications currently tested.

### dNs and CsH in Unstimulated mPB HSPC Allow to Reach Good Level of Transduction While Preserving Their Repopulation Capacity

On these premises, we decided to confirm the potential of our combinatorial transduction protocol in a clinically relevant setting, transplanting human mobilized peripheral blood (mPB)-derived HSPC.

Unstimulated mPB-derived HSPC were transduced with LV at MOI 50 in presence of CsH and dNs. As a control, HSPC were pre-stimulated overnight and transduced in presence of CsH alone. Cells were then transplanted into NSG mice by t₀ equivalent and engraftment and transduction efficiency were followed in vivo over time (FIG. 9A). From the in vitro analysis we confirmed high gene marking levels in unstimulated mPB-CD34+ cells transduced in presence of dNs and CsH, reaching 60% of GFP⁺ cells in the bulk and in the different HSPC subpopulations (FIG. 9B-C), with no major impact observed on HSPC composition (FIG. 9D). The transduction level was confirmed in terms of integrated viral copies despite overall integrated copies in unstimulated cells remained lower compared to CsH-stimulated HSPC, as previously observed in CB-CD34+ cells (FIG. 8E). Despite lower initial cellular input (FIG. 9F), unstimulated HSPC showed similar engraftment compared to their stimulated counterpart at all the time points analyzed (FIG. 9G). Similar levels of engraftment were confirmed from the analysis of the bone marrow at 20 weeks post-transplantation (FIG. 9H). Importantly, we confirmed that with our transduction protocol we could achieve and maintain high level of transduction in vivo also in the mPB source of unstimulated HSPC (FIG. 9I). The high transduction levels were also confirmed in the different organs at 20 weeks post-transplantation (FIG. 9J-L). Also in this context, the number of integrated copies in vivo remained lower in the unstimulated group if compared to CsH-stimulated one (FIG. 9M-O). This again reflects the fact that we are using as a reference for stimulated cells a protocol which have been demonstrated to be superior to the current clinical reference protocol. Indeed, the number of copies obtained in vivo from unstimulated HSPC transduced with our combinatorial protocol are in line or even higher than the number of copies usually retrieved from a standard II-hit clinical protocol in stimulated HSPC using clinical grade lentiviral vectors.

Overall, our data demonstrated that unstimulated HSPC engraft and yield hematopoietic output at similar levels compared to their stimulated counterparts despite lower initial cellular input. This suggests better preservation of their biological properties and repopulation capacity due to the lack of ex vivo culture. Importantly, with our transduction protocol we could reach high levels of transgene expression and clinically relevant levels of integrated copies long term in vivo without affecting HSC function. This is extremely important if we consider some of the limitations encountered in current clinical trials. Thus far, X-linked chronic granulomatous disease (X-CGD) LV gene therapy has failed to fully restore functional defects and several patients have lost engraftment of gene-marked HSC. While etiology of graft loss remains unclear, increased oxidative DNA damage and inflammation have been suggested to compromise X-CGD HSC. Similarly, current gene therapy protocols for Fanconi Anemia disease rely on very short, and thus less efficacious, transduction protocols aimed at limiting as much as possible the ex-vivo manipulation of these extremely fragile cells. On these premises, we predict that our CsH+dNs transduction protocol that does not require prolonged ex vivo culture of HSPC will provide a significant improvement for such settings in which the disease background impacts HSC biological properties.

### Example 4—Further Materials and Methods

### Vectors

For pseudotyping LV with measles envelope, pMD2.VSV-g was replaced by the Measles envelope encoding plasmids during vector production as previously described (Girard-Gagnepain et al. (2014) Blood 124: 1221-31). The SIN-RV was produced as previously described (Montini et al. (2006) Nat Biotechnol 24: 687-96) using as transfer vector RVrkat43.2MLV GFP, the packaging plasmid pCM-gag-pol and the VSV-G envelop-encoding pMD2.VSV-G plasmid. Simian immunodeficiency virus macaque—(SIVmac) based vectors were produced as previously described (Mangeot et al. (2002) Mol Ther 5: 283-90) using an GFP encoding genome SIVmac-GFP, SIVmac packaging plasmid SIV3+ and VSV-G pseudotyped.

### Compounds

Uridine 5′-monophosphate (UMP) was purchased from Selleckchem (Catalog No. S9451), resuspended and stored following the manufacturer's instructions. Where indicated, UMP (1 mM) was added to the culture media 4 h-2 h before transduction and washed out with the vector 16-20 hours later. Orotic acid was purchased from Sigma (Catalog No. O2750) resuspended and stored following the manufacturer's instructions. Where indicated, orotic acid (7.5 μM) was added to the culture media 4 h-2 h before transduction and washed out with the vector 16-20 hours later.

RNA Extraction, qPCR and Gene Expression Analysis

The following Taqman probes from Applied Biosystems were additionally used: DHODH (Hs00361406_m1); CPS II (Hs00983188_m1).

### Colony-forming Cell Assay

Colony-forming cell assays were performed by plating 8×10² human HSPC transduced in presence of the different compounds in a methylcellulose-based medium (Methocult GF4434; Stem Cell Technologies). Fifteen days later colonies were scored by light microscopy for colony numbers and morphology as erythroid or myeloid. Moreover, they were collected as a pool and as a single colony, and lysed for molecular analysis to evaluate transduction efficiencies with clinical grade LV.

Apoptosis assay

The apoptosis assays were performed with the Annexin V Apoptosis Detection Kit I (BD Pharmigen, RRID:AB_1279044) according to the manufacturer's instructions and 48 hours after dNs addition and/or transduction.

### Cell Proliferation Assay

Cells were stained with Cell Proliferation Dye eFluor@670 (Affimetrix, eBioscience) immediately after thawing. This fluorescent dye binds to any cellular protein containing primary amines, and as cells divide, the dye is distributed equally between daughter cells that can be measured as successive halving of the fluorescence intensity of the dye. At different time points after dNs addition/transduction, cells were harvested and analyzed at flow cytometry.

1. A combination of: (a) at least one deoxyribonucleoside (dN) or a derivative thereof and cyclosporin H (CsH) or a derivative thereof; or (b) at least one pyrimidine precursor and cyclosporin H (CsH) or a derivative thereof.

2. The combination according to claim 1, wherein the at least one dN or a derivative thereof comprises at least one pyrimidine dN or a derivative thereof.

3. The combination according to claim 1 or 2, wherein the at least one dN or a derivative thereof comprises deoxycytidine (dC) or a derivative thereof and/or thymidine (dT) or a derivative thereof, preferably wherein the at least one dN or a derivative thereof comprises dC or a derivative thereof.

4. The combination according to claim 2 or 3, wherein the at least one dN or a derivative thereof further comprises at least one purine dN or a derivative thereof.

5. The combination according to any of claims 2 to 4, wherein the at least one dN or a derivative thereof further comprises deoxyadenosine (dA) or a derivative thereof and/or deoxyguanosine (dG) or a derivative thereof.

6. The combination according to any preceding claim, wherein the at least one dN or a derivative thereof comprises or consists of dC or a derivative thereof, dT or a derivative thereof, dA or a derivative thereof, and dG or a derivative thereof.

7. The combination according to any preceding claim, wherein at least one dN or a derivative thereof and CsH or a derivative thereof are in a dN:CsH molar ratio of from about 2:1 to about 200:1, preferably wherein at least one dN or a derivative thereof and CsH or a derivative thereof are in a dN:CsH molar ratio of from about 10:1 to about 100:1.

8. The combination according to any preceding claim, wherein at least one pyrimidine dN or a derivative thereof and CsH or a derivative thereof are in a dN:CsH molar ratio of from about 2:1 to about 200:1, preferably wherein at least one pyrimidine dN or a derivative thereof and CsH or a derivative thereof are in a dN:CsH molar ratio of from about 10:1 to about 100:1.

9. The combination according to any preceding claim, wherein dC or a derivative thereof and CsH or a derivative thereof are in a dN:CsH molar ratio of from about 2:1 to about 200:1 and/or dT or a derivative thereof and CsH or a derivative thereof are in a dN:CsH molar ratio of from about 2:1 to about 200:1, preferably wherein dC or a derivative thereof and CsH or a derivative thereof are in a dN:CsH molar ratio of from about 10:1 to about 100:1 and/or dT or a derivative thereof and CsH or a derivative thereof are in a dN:CsH molar ratio of from about 10:1 to about 100:1.

10. The combination according to claim 8 or 9, wherein dA or a derivative thereof and/or dG or a derivative thereof are in a dN:CsH molar ratio of from about 2:1 to about 200:1, preferably wherein the dA or a derivative thereof and/or dG or a derivative thereof are in a dN:CsH molar ratio of from about 10:1 to about 100:1.

11. The combination according to any preceding claim, wherein at least one dN or a derivative thereof is at a concentration of from about 25 μM to about 1000 μM, preferably from about 100 UM to about 500 μM.

12. The combination according to any preceding claim, wherein at least one pyrimidine dN or a derivative thereof is at a concentration of from about 25 μM to about 1000 μM, preferably from about 100 μM to about 500 μM.

13. The combination according to any preceding claim, wherein dC or a derivative thereof is at a concentration of from about 25 μM to about 1000 μM, preferably from about 100 μM to about 500 μM, and/or dT or a derivative thereof is at a concentration of from about 25 μM to about 1000 μM, preferably from about 100 μM to about 500 μM.

14. The combination according to claim 12 or 13, wherein dA or a derivative thereof is at a concentration of from about 25 μM to about 1000 μM, preferably from about 100 μM to about 500 μM, and/or dG or a derivative thereof is at a concentration of from about 25 μM to about 1000 μM, preferably from about 100 μM to about 500 μM.

15. The combination according to any preceding claim, wherein dC or a derivative thereof is at a concentration of from about 25 μM to about 1000 μM, dT or a derivative thereof is at a concentration of from about 25 μM to about 1000 μM, dA or a derivative thereof is at a concentration of from about 25 μM to about 1000 μM, and dG or a derivative thereof is at a concentration of from about 25 μM to about 1000 μM.

16. The combination according to any preceding claim, wherein CsH or derivative thereof is at a concentration of from about 1 μM to about 50 μM.

17. The combination according to any preceding claim, wherein the pyrimidine precursor is orotic acid (OA) or uridine 5′-monophosphate (UMP).

18. A composition comprising a combination according to any of claims 1 to 17, optionally wherein the composition is a cell culture medium or a media supplement, preferably wherein the composition is a cell culture medium.

19. A kit comprising a combination according to any of claims 1 to 17 or a composition according to claim 18, and optionally one or more further compositions for cell transduction and/or optionally one or more further agents for cell transduction.

20. Use of a combination according to any of claims 1 to 17, a composition according to claim 18, or a kit according to claim 19, for increasing the efficiency of transduction of an isolated population of cells by a viral vector and/or increasing the efficiency of gene editing of an isolated population of cells when transduced by a viral vector.

21. A method of transducing a population of cells comprising the steps of:
(a) contacting the population of cells with a combination according to any of claims 1 to 17 or a composition according to claim 18; and
(b) transducing the population of cells with a viral vector.

22. The method of claim 21, wherein the method is an in vitro method or an ex vivo method.

23. Use of (a) a deoxyribonucleoside (dN) or a derivative thereof; or (b) at least one pyrimidine precursor for increasing the efficiency of transduction of an isolated population of cells by a viral vector and/or increasing the efficiency of gene editing of an isolated population of cells when transduced by a viral vector.

24. A method of transducing a population of cells comprising the steps of:
(a) contacting the population of cells with (I) at least one deoxyribonucleoside (dN) or a derivative thereof or (II) at least one pyrimidine precursor; and
(b) transducing the population of cells with a viral vector;
wherein the population of cells comprises or consists substantially of: (i) unstimulated haematopoietic stem and/or progenitor cells (HSPCs); and/or (ii) CD14⁻ peripheral blood mononuclear cells (PBMCs).

25. The method of claim 24, wherein the method is an in vitro method or an ex vivo method.

26. The use according to claim 23, or the method according to claim 24 or 25, wherein the dN or a derivative thereof is a pyrimidine dN or a derivative thereof.

27. The use according to claim 23 or 26, or the method according to any of claims 24 to 26, wherein the dN or a derivative thereof is deoxycytidine (dC) or a derivative thereof and/or thymidine (dT) or a derivative thereof, preferably wherein the dN or a derivative thereof is deoxycytidine (dC) or a derivative thereof.

28. The use according to any of claim 23, 26 or 27, or the method according to any of claims 24 to 27, wherein the dN or a derivative thereof is at a concentration of from about 25 μM to about 1000 μM, preferably wherein the dN or a derivative thereof is at a concentration of from about 100 μM to about 500 μM.

29. The use according to any of claims 23, 26 to 28, or the method according to any of claims 24 to 28, wherein the population of cells is contacted with (a) the dN or a derivative thereof in combination with CsH or a derivative thereof; or (b) the pyrimidine precursor in combination with CsH or a derivative thereof.

30. The use according to claim 29, or the method according to claim 29, wherein the CsH or derivative thereof is at a concentration of from about 1 to about 50 μM.

31. The use according to any of claims 23 or 26 to 30, or the method according to any of claims 24 to 30, wherein the pyrimidine precursor is orotic acid (OA) or uridine 5′-monophosphate (UMP).

32. The use according to any of claims 26 to 31, or the method according to any of claims 26 to 31, wherein the population of cells is further contacted with a purine dN or a derivative thereof.

33. The use according to claim 32, or the method according to claim 32, wherein the purine dN or a derivative thereof is at a concentration of from about 25 μM to about 1000 μM, preferably wherein the purine dN or a derivative thereof is at a concentration of from about 100 μM to about 500 μM.

34. The use according to any of claims 20, 23, 26 to 33, or the method according to any of claims 21, 22, 24 to 33, wherein the cells are human cells or mouse cells, preferably human cells.

35. The use according to any of claims 20, 23, 26 to 34, or the method according to any of claims 21, 22, 24 to 34, wherein the cells are quiescent cells.

36. The use according to any of claims 20, 23, 26 to 35, or the method according to claim 21 or 22, wherein the population of cells comprises or consists substantially of:
(i) haematopoietic stem and/or progenitor cells (HSPCs); and/or
(ii) CD14⁻ peripheral blood mononuclear cells (PBMCs).

37. The use according to claim 36, or the method according to any of claims 24 to 36, wherein the HSPCs are unstimulated HSPCs.

38. The use according to claim 36 or 37, or the method according to any of claims 24 to 37, wherein the HSPCs are CD34⁺ or CD34⁻ cells, preferably wherein the CD34⁺ cells are CD34⁺CD133⁻CD90⁻, CD34⁺CD133⁺CD90⁻, or CD34⁺CD133⁺CD90⁺ cells, more preferably wherein the CD34⁺ cells are CD34⁺CD133⁺CD90⁺ cells.

39. The use according to any of claims 36 to 38, or the method according to any of claims 24 to 38, wherein the PBMCs are CD3⁺, CD4⁺, and/or CD8⁺ T cells.

40. The use according to any of claims 36 to 39, or the method according to any of claims 24 to 39, wherein the method further comprises a step of enriching the population of cells for the HSPCs or CD14⁻ PBMCs.

41. The use according to any of claims 20, 23, 26 to 40, or the method according to any of claims 21, 22, 24 to 40, wherein the viral vector is a retroviral vector, preferably a lentiviral vector.

42. The use according to claim 41, or the method according to claim 41, wherein the lentiviral vector is an integration-defective lentiviral vector.

43. The use according to any of claims 20, 23, 26 to 42, or the method according to any of claims 21, 22, 24 to 42, wherein the percentage of cells transduced by the vector is increased and/or the vector copy number per cell is increased.

44. A method of gene therapy comprising the steps of:
(a) transducing a population of cells according to the method of any of claims 21, 22, 24 to 43; and
(b) administering the transduced cells to a subject.

45. The method of claim 44, wherein the transduced cells are administered to a subject as part of an autologous stem cell transplant procedure or an allogeneic stem cell transplant procedure.

46. A population of cells prepared according to the method of any of claims 21, 22, 24 to 43.

47. A pharmaceutical composition comprising the population of cells of claim 46.

48. The population of cells of claim 46 for use in therapy.

49. The population of cells for use according to claim 48, wherein the population is administered as part of an autologous stem cell transplant procedure or an allogeneic stem cell transplant procedure.

50. The combination according to any of claims 1 to 17, the composition according to claim 18, or the kit according to claim 19, for use in gene or cell therapy.

51. A deoxyribonucleoside (dN) or a derivative thereof for use in gene or cell therapy.

52. A pyrimidine precursor for use in gene or cell therapy.


SEQUENCE LISTING (3 sequences)

SEQ ID NO: 1 - DNA, 22 bp, Artificial Sequence
  Synthetic
tcaccaatcctgtccctagtgg

SEQ ID NO: 2 - DNA, 18 bp, Artificial Sequence
  Synthetic
gtccctcttcgggcgcca

SEQ ID NO: 3 - DNA, 19 bp, Artificial Sequence
  Synthetic
  misc_feature (19)..(19): n is a, c, g, or t
cagggagaagcccgcggtn